(19) United States
(12) Patent Application Publication
(10) Pub. No.: US 2025/0164502 A1
(43) Pub. Date: May 22, 2025

(54) Mutant Human Nax Proteins and Screening Methods

(71) Applicants: Genentech, Inc., South San Francisco, CA (US); University of Copenhagen, København (DK)

(72) Inventors: Cameron L. Noland, San Francisco, CA (US); Jian Mehr-Dean Payandeh, Belmont, CA (US); Stephan Alexander Pless, Copenhagen (DK); Han Chow Chua, Copenhagen (DK); Marc Kschonsak, South San Francisco, CA (US)

(73) Assignees: Genentech, Inc., South San Francisco, CA (US); University of Copenhagen, København (DK)

(21) Appl. No.: 18/515,382

(22) Filed: Nov. 21, 2023

Related U.S. Application Data
(63) Continuation of application No. PCT/US2022/031302, filed on May 27, 2022.
(60) Provisional application No. 63/195,039, filed on May 30, 2021.

(51) Int. Cl. G01N 33/68; C07K 14/47

(52) U.S. Cl. CPC G01N 33/6872; C07K 14/4702; G01N 2333/4703; G01N 2500/04

(57) ABSTRACT

The present disclosure relates to mutant human Na_{X} ion channel (“Na_{X}”) proteins and screening methods using a mutant human Na_{X} that may be used to identify molecules that modulate the activity of human Na_{X}. For example, in some embodiments, screening methods involve performing an ion channel assay on a mutant human Na_{X} in the presence of a potential Na_{X} modulator. The disclosure also relates to molecules that act as modulators of human Na_{X} and associated kits for detecting such molecules.

### CROSS-REFERENCE TO RELATED APPLICATIONS

This application is a continuation of International Patent Application No. PCT/US2022/031302, filed May 27, 2022, which claims the benefit of priority of U.S. Provisional Application No. 63/195,039, filed May 30, 2021, the contents of each of which are incorporated by reference herein in their entireties for any purpose.

### SEQUENCE LISTING

The instant application contains a Sequence Listing which has been submitted electronically in XML format and is hereby incorporated by reference in its entirety. Said XML copy, created on Nov. 14, 2023, is named “01164-0016-00US.xml” and is 178,992 bytes in size.

### FIELD

The present disclosure relates to mutant human Na_{X} ion channel (“Na_{X}”) proteins and screening methods using a mutant human Na_{X} that may be used to identify molecules that modulate the activity of human Na_{X}. For example, in some embodiments, screening methods involve performing an ion channel assay on a mutant human Na_{X} in the presence of a potential Na_{X} modulator. The disclosure also relates to molecules that act as modulators of human Na_{X} and associated kits for detecting such molecules.

### BACKGROUND

Prototypical voltage-gated sodium (Na_{V}) channels perform key roles in electrical signaling by initiating and propagating action potentials. Nine closely related mammalian channel subtypes, Na_{V}1.1-Na_{V}1.9, are expressed in a range of tissues where they contribute to essential physiological processes including nervous system function, muscle contraction, and hormone secretion. A tenth Na_{V} channel-like gene, SCN7A, was cloned nearly three decades ago, but atypical sequence features and the inability to record voltage-activated sodium (Na⁺)-currents from this Na_{V}2.1 protein raised speculation that it might have distinct physiological roles, resulting in the designation Na_{X}.

In contrast to canonical Na_{V} channels, Na_{X} has been proposed to function as a Na⁺-channel activated by the extracellular Na⁺ concentration. Multiple lines of evidence suggest that Na_{X} may contribute to Na⁺ homeostasis^{1,2}. Na_{X} shows restricted expression in a brain area that specializes in monitoring blood composition⁵. Na_{X}-knockout mice ingest salt despite dehydration and are resistant to hypertension caused by elevated blood Na⁺ levels^{5,6}. In humans, autoimmunity against Na_{X} causes chronic hypernatremia with impaired thirst perception and salt appetite⁷. In the periphery, Na_{X} regulates Na⁺ homeostasis upstream of the epithelial Na⁺ channel (ENaC) and has been suggested as a target to treat atopic dermatitis and hypertrophic scarring⁸.

Na_{X} shares highest sequence identity (˜55%) with the Na_{V}1.7 channel¹⁶ but it is the most divergent member of the Na_{V} channel family. In conventional Na_{V} channels, the voltage sensor domains (VSDs) contain 4-8 gating charge residues conserved along the S4 segment that undergo outward movement upon membrane depolarization to open the channel gate (VSD1-VSD3) or initiate fast inactivation (VSD4). In contrast to this paradigm, Na_{X} has been reported to be voltage-insensitive and contain a reduced number of S4 gating charge residues. Thus, the function and conformational state of the voltage sensor-like domains (VSLDs) in Na_{X} is unclear, and how the VSLDs contribute to channel gating is unknown. In addition, the DIII-DIV linker segment, which is divergent in Na_{X} in comparison to Na_{V}, is known to be required for fast inactivation in Na_{V} channels.

### SUMMARY

The present disclosure relates to mutant human Na_{X} ion channel (“Na_{X}”) proteins and screening methods using a mutant human Na_{X} that may be used to identify molecules that modulate the activity of human Na_{X}, and to molecules identified by such methods and kits for performing the methods. The mutant human Na_{X} proteins and associated methods may be used, for example, to identify molecules that not only modulate Na_{X} activity, but also to test molecules known to bind to or modulate Na_{V} proteins to test their selectivity to their target proteins. Modulators identified in assays described herein using a mutant Na_{X}, may be useful as modulators/candidate modulators of human Na_{X}. The present inventors have surprisingly found mutant Na_{X} proteins that allow ion channel measurements relevant to human Na_{X} by mimicking an open, conductive state of the ion channel. Without being bound by theory, mutant Na_{X} may mimic an open, conductive state of the Na_{X} ion channel by mimicking, e.g., phosphorylation of the ion channel protein(s) and/or other relevant physiological or structural changes that normally allow the Na_{X} ion channel to open. The present disclosure includes, for example, any one or a combination of the following embodiments.

In some embodiments, the disclosure herein relates to methods of identifying a human Na_{X} ion channel protein (Na_{X}) modulator, comprising: (a) providing a mutant human Na_{X} in which at least a portion of the DIII domain of human Na_{X} is replaced by a corresponding portion of the DIII domain of a human or mammalian Na_{V} protein (Na_{V}); (b) performing an ion channel assay on the mutant human Na_{X} in the presence of a potential Na_{X} modulator; and (c) identifying the potential modulator as a human Na_{X} modulator if the activity of the mutant human Na_{X} in the assay in the presence of the potential modulator is higher or lower than the activity in the absence of the potential modulator. In some cases, the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5 and/or does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a corresponding portion of mammalian Na_{V}1.1, mammalian Na_{V}1.2, mammalian Na_{V}1.3, mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. For example, in some cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20.

The present disclosure also encompasses methods of identifying a human Na_{X} ion channel protein (Na_{X}) modulator, comprising: (a) providing a mutant human Na_{X} comprising a substitution of at least one residue on each of two, three, or four S6 alpha helices of human Na_{X} with a glycine, proline, or polar or charged residue; (b) performing an ion channel assay on the mutant human Na_{X} in the presence of the potential modulator; and (c) identifying the potential modulator as a human Na_{X} modulator if the activity of the mutant human Na_{X} in the assay in the presence of the potential modulator is higher or lower than the activity in the absence of the potential modulator. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three or more of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}.

In any of the above methods, the ion channel assay may also be performed in the presence of an identified modulator of the mutant human Na_{X}, for example, a modulator identified herein or otherwise identified in performance of the methods herein. In some such cases, the identified modulator of the mutant human Na_{X} is one or more of tetrodotoxin (TTX), quinidine, flecainide, tetracaine, or lidocaine.

In any of the above methods, the potential modulator may be a peptide or macrocycle or antibody. For example, in some cases, the potential modulator is a small molecule. In some cases, a small molecule may be a derivative of tetrodotoxin (TTX), quinidine, flecainide, tetracaine, or lidocaine, for example.

Any of the above methods may also further comprise determining the binding affinity of the potential modulator identified in part (c) to either or both of the mutant human Na_{X} and wild-type human Na_{X}. In some cases, the potential modulator binds to either or both of the mutant human Na_{X} and wild-type human Na_{X} an EC50 or IC50 of 10 μM or less, 10 μM to 50 nM, 10 M to 500 nM, 1 μM or less, 1 μM to 50 nM, or 100 nM or less. In some cases, an EC50 or IC50 may be determined in an ELISA assay.

In any of the above methods, the ion channel assay may be a patch clamp or an automated patch clamp assay, an ion flux assay, or an ion- or voltage-sensitive dye assay. In any of the above methods, the potential modulator identified in part (c) may reduce the activity of the mutant human Na_{X} in the assay by at least 25%, at least 30%, at least 40%, at least 50%, at least 60%, at least 70%, at least 80%, or at least 90%. In some cases, the potential modulator identified in part (c) reduces the activity of the mutant human Na_{X} in the assay with a half-maximal concentration of 10 nM to 500 μM, 50 nM to 500 μM, 10 nM to 50 μM, 100 nM to 500 μM, 100 nM to 50 μM, 1-500 μM, 1-50 μM, 10-500 μM, or 50-250 μM. In other cases, the potential modulator identified in part (c) increases the activity of the mutant human Na_{X} in the assay.

The present disclosure also relates to modulators of human Na_{X} identified by the methods described herein, which may be optionally peptides, macrocycles, small molecules, or antibodies.

The present disclosure also encompasses mutant human Na_{X} ion channel (Na_{X}) proteins, wherein at least a portion of the DIII domain of human Na_{X} is replaced by a corresponding portion of the DIII domain of a human or mammalian Na_{V} protein (Na_{V}). In some cases, the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5 and/or does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a corresponding portion of mammalian Na_{V}1.1, mammalian Na_{V}1.2, mammalian Na_{V}1.3, mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. For example, in some cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20.

The present disclosure further includes mutant human Na_{X} ion channel (Na_{X}) proteins, comprising a substitution of at least one residue on each of the two, three, or four S6 alpha helices with a glycine, proline, or polar or charged residue. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three or more of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}.

The present disclosure further includes methods of determining whether a test molecule that modulates the activity of a human ion channel protein modulates the activity of human Na_{X} ion channel (Na_{X}) protein, comprising: (a) providing a mutant human Na_{X} in which at least a portion of the DIII domain of human Na_{X} is replaced by a corresponding portion of the DIII domain of a human or mammalian Na_{V} protein (Na_{V}); (b) performing an ion channel assay on the mutant human Na_{X} in the presence of the test molecule; and (c) determining that the test molecule is a human Na_{X} modulator if the activity of the mutant human Na_{X} in the assay in the presence of the test molecule is higher or lower than the activity in the absence of the test molecule; and optionally, (d) selecting the test molecule for additional screening if it is not a human Na_{X} modulator according to part (c).

The present disclosure additionally includes methods of determining whether a test molecule that modulates the activity of a human ion channel protein modulates the activity of human Na_{X} ion channel (Na_{X}) protein, comprising: (a) determining that the test molecule modulates the activity of the human ion channel protein; (b) providing a mutant human Na_{X} in which at least a portion of the DIII domain of human Na_{X} is replaced by a corresponding portion of the DIII domain of a human or mammalian Na_{V} protein (Na_{V}); (c) performing an ion channel assay on the mutant human Na_{X} in the presence of the test molecule; and (d) determining that the test molecule is a human Na_{X} modulator if the activity of the mutant human Na_{X} in the assay in the presence of the test molecule is higher or lower than the activity in the absence of the test molecule; and optionally, (e) selecting the test molecule for additional screening if it is not a human Na_{X} modulator according to part (d).

In the two additional methods above, in some cases the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5 and/or does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. In some such cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20.

The present disclosure also includes methods of determining whether a test molecule that modulates the activity of a human ion channel protein modulates the activity of human Na_{X} ion channel (Na_{X}) protein, comprising: (a) providing a mutant human Na_{X} comprising a substitution of at least one residue on each of two, three, or four S6 alpha helices of human Na_{X} with a glycine, proline, or polar or charged residue; (b) performing an ion channel assay on the mutant human Na_{X} in the presence of the test molecule; and (c) determining that the test molecule is a human Na_{X} modulator if the activity of the mutant human Na_{X} in the assay in the presence of the test molecule is higher or lower than the activity in the absence of the test molecule; and optionally, (d) selecting the test molecule for additional screening if it is not a human Na_{X} modulator according to part (c).

The disclosure further encompasses methods of determining whether a test molecule that modulates the activity of a human ion channel protein also modulates the activity of human Na_{X} ion channel (Na_{X}) protein, comprising: (a) determining that the test molecule modulates the activity of the human ion channel protein; (b) providing a mutant human Na_{X} comprising a substitution of at least one residue on each of two, three, or four S6 alpha helices of human Na_{X} with a glycine, proline, or polar or charged residue; (c) performing an ion channel assay on the mutant human Na_{X} in the presence of the test molecule; and (d) determining that the test molecule is a human Na_{X} modulator if the activity of the mutant human Na_{X} in the assay in the presence of the test molecule is higher or lower than the activity in the absence of the test molecule; and optionally, (e) selecting the test molecule for additional screening if it is not a human Na_{X} modulator according to part (d).

In either of the two methods above, in some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three or more of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}.

The present disclosure also includes molecules identified by the methods above, which optionally may be peptides, macrocycles, small molecules, or antibodies.

The disclosure also includes molecular complexes comprising a chimeric or mutant human Na_{X} protein as described herein and a modulator of the protein or a potential modulator of the protein.

The disclosure also includes kits for assaying the activity of human Na_{X} or for identifying a human Na_{X} modulator and/or a molecule that binds to human Na_{X}, comprising a mutant human Na_{X} as described herein, and further comprising at least one of: one or more reagents for conducting an ion channel assay, a modulator of the mutant human Na_{X}, and instructions for use.

### BRIEF DESCRIPTION OF THE DRAWINGS

FIGS. 1A-H show characterization of human Na_{X} and overall structure of the β3-Na_{X} channel complex. FIG. 1A shows representative currents from Xenopus laevis oocytes expressing human Na_{X} or Na_{V}1.7. FIG. 1B shows representative currents from oocytes expressing Na_{X} in response to extracellular application of indicated compounds. FIG. 1C shows representative currents from oocytes expressing Na_{X} and co-expression of Na_{V} or Ca_{V} channel auxiliary subunits, Na⁺/K⁺-ATPase subunits and synapse-associated protein 97 (SAP97), and in the presence of the indicated extracellular Na⁺ concentration. Voltage protocols for FIGS. 1A-C are as follows: Na_{X}: steps between +80 to −100 mV, in 20 mV increments from a HP of 0 mV; Na_{V}1.7: depolarizing steps between −80 and +65 mV, in 5 mV increments, from a HP of −100 mV. FIG. 1D shows data summary of independent experiments performed as in FIGS. 1A-C. FIG. 1E shows representative currents from murine Neuro-2 cells expressing human Na_{X} or Na_{V}1.7 in response to changes of the extracellular Na⁺ concentration (HP=−60 mV), as indicated, or voltage: depolarizing steps between −60 to +60 mV, in 10 mV increments from a HP of −100 mV. FIG. 1F shows data summary of independent experiments performed as in FIG. 1E. FIG. 1G shows Western blots of total lysate and surface fraction of proteins extracted from Neuro-2 cells probed for the indicated proteins. FIG. 1H shows side and extracellular view of the β3-Na_{X} channel complex. Approximate membrane boundaries are indicated. DI, DII, DIII and DIV are colored in green, blue, orange and pink, respectively, with the β3 subunit in grey surface representation.

FIGS. 2A-E show structure of the Na_{X} pore module reveals a non-conductive state. FIG. 2A show Na_{X} pore volume is shown as grey surface with DII and DIV shown in cartoon rendering (DI and DIII omitted for clarity). FIG. 2B show view of the S6-helices with side-chains lining the activation gate in stick and semi-transparent surface representation. Orthogonal view provides a wider perspective with DIII and DIV colored orange and pink, with the IFI motif (green, in dashed box) from the DIII-DIV linker shown in stick and semi-transparent surface representation. FIG. 2C show orthogonal views sliced through the pore module highlighting lateral fenestrations and bound lipids. The phosphatidylethanolamine that crosses the S6-gate is in purple stick representation. In side view, a lipid in the foreground has been removed for clarity. The view in FIG. 2D is similar to middle panel C, but with cryo-EM map shown around modeled lipids in blue mesh representation. FIG. 2E shows representative currents from Xenopus laevis oocytes expressing human Na_{X}, human Na_{V}1.7 or double- and single-domain-swapped Na_{V}1.7-Na_{X} chimeras, including chimera constructs 1-7 shown in Table 4 herein. Steps between +80 to −100 mV, in 20 mV increments, from a HP of 0 mV.

FIGS. 3A-F show characterization of human Na_{X} carrying targeted pore-wetting S6-mutations. FIG. 3A shows location and zoomed view of targeted hydrophobic side-chains lining the S6-gate. FIG. 3B shows representative currents from Xenopus laevis oocytes expressing the Na_{X}-QTT construct under indicated voltage protocols. FIG. 3C shows representative currents from oocytes expressing the indicated Na_{X} construct. FIG. 3D shows representative currents from oocytes expressing the Na_{X}-EEE construct. Voltage protocols for FIGS. 3C-D were as above in FIG. 2E. FIG. 3E shows representative currents from oocytes expressing the Na_{X}-QTT construct and co-expressing indicated Na_{V} auxiliary subunit. Voltage protocols as above. FIG. 3F shows data summary of independent experiments performed in FIGS. 2B-E.

FIGS. 4A-C show structure and characterization of the Na_{X} selectivity filter. FIG. 4A shows Na_{X} DENA-motif residue side-chains shown as sticks. FIG. 4B, top panels, shows Na_{X} side-chains and analogous residues that form an interaction network around the selectivity filter in Na_{V} channels are shown in stick representation (Na_{V}1.7, PDB 6J8J). FIG. 4B, bottom panels show the same view of the Na_{X} and Na_{V}1.7 selectivity filters (as in FIG. 4B, top panels) but as electrostatic surface rending. Central cavity and activation gate excluded for clarity. FIG. 4C shows superimposed and zoomed view of the DI-DIV interface comparing Na_{X} and Na_{V}1.7 (grey, PDB 6J8J) structures with select side-chains shown as sticks. FIG. 4D shows representative currents from HEK293 cells expressing human Na_{X}-QTT with a C-terminal GFP-Flag tag in a physiological (FIG. 4D, left panel) or NMDG⁺-only extracellular solution (FIG. 4D, middle panel). Steps between +80 to −100 mV, in 20 mV increments, from a HP of 0 mV.

FIG. 4D, right panel, shows I-V curve data summary from independent experiments. FIG. 4E, left panel, shows representative currents from HEK293 cells expressing human Na_{X}-QTT with indicated monovalent cations in the extracellular solution. Voltage ramp from −80 to +80 mV was applied. FIG. 4E, right panel, shows summary of reversal potentials and permeability ratios measured from independent experiments.

FIGS. 5A-C show pharmacology of the human Na_{X}-QTT channel. FIG. 5A, left and middle panels, show representative I-V currents from HEK293 cells expressing human Na_{X}-QTT with indicated extracellular monovalent cations with or without indicated amounts of CaCl₂ in the extracellular solution. Voltage ramp from −80 to +80 mV was applied. FIG. 5A, right panel, shows percentage of block of outward Na⁺ by indicated concentrations of Ca²⁺ at 80 mV. FIG. 5B shows representative currents from Xenopus laevis oocytes expressing human Na_{X}-QTT in standard extracellular solution with or without indicated divalent and trivalent cations (unit in mM), when stepping from 0 to +80 mV. FIG. 5C shows representative currents from oocytes expressing human Na_{X}-QTT in standard extracellular solution with or without indicated blockers added, when stepping from 0 to +80 mV (left panel) or from 0 to −100 mV (right panel). FIG. 5C, middle panel, shows summary of currents measured from independent experiments.

FIGS. 6A-E show structure of the atypical Na_{X} voltage sensor-like domains. FIG. 6A show VSLD1 with gating charges (blue), HCS (green), and ENC/INC (red) side-chains shown in stick representation. Inset highlights a unique proline in Na_{X}, with Na_{V}1.7 VSD1 (PDB 6J8J) superimposed for comparison. FIG. 6B show VSLD2 rendered as in FIG. 6A. Insets highlight His579-S4 interaction (FIG. 6B, top panel) and S4-S5 linker displacements (FIG. 6B, bottom panel), with Na_{V}1.7 DII (PDB 6J8J) superimposed for comparison. FIG. 6C shows VSLD3 rendered as in FIG. 6A. Inset highlights Phe1011 from the S3 helix. FIG. 6D shows VSLD4, rendered as in FIG. 6A. Inset shows a comparison of the S4/S4-S5 linker region between Na_{X} and Na_{V}1.7 VSD4 (PDB 6J8J), with Ca of gating charge residues indicated as blue spheres. FIG. 6E shows representative currents from Xenopus laevis oocytes expressing Na_{V}1.7 and various Na_{V}1.7-Na_{X}-VSLD in response to depolarizing steps between −80 and +65 mV in 5 mV increments from a HP of −100 mV.

FIGS. 7A-F show functional evaluation of human Na_{X} in different expression systems. FIG. 7A shows Western blots of total lysate and surface fraction of proteins extracted from HEK293T cells expressing the indicated constructs. FIG. 7B shows representative currents from HEK293T cells expressing human Na_{X} or Na_{V}1.7 (with C-terminal GFP and Flag tags) with indicated voltage protocol. FIG. 7C shows Western blots of total lysate and surface fraction of proteins extracted from Xenopus laevis oocytes expressing the indicated constructs. FIG. 7D shows representative currents from oocytes expressing human Na_{X} or Na_{V}1.7 with indicated voltage protocol. FIG. 7E shows Western blots of total lysate and surface fraction of proteins extracted from murine Neuro-2A cells expressing the indicated constructs. FIG. 7F shows representative currents from Neuro-2A cells expressing human Na_{X} (*, with C-terminal GFP and Flag tags) under indicated extracellular Na⁺ concentrations (HP=−60 mV). Seal resistances (Rm) of individual cells are provided.

FIGS. 8A-D show functional evaluation of human Na_{X} in Xenopus laevis oocytes. FIGS. 8A, left panel, and 8B, left panel, show representative currents from Xenopus laevis oocytes expressing human Na_{X} with the indicated pharmacological modulator present in the extracellular solution. FIG. 8A, right panel, shows I-V plots with depolarizing steps between +80 and −100 mV, in 20 mV increments, from a HP of 0 mV. FIG. 8B, left panel, shows I-V plots with depolarizing steps between −100 and +100 mV, in 20 mV increments, from a HP of −100 mV. FIGS. 8C, left panel, and 8D, left panel, show representative currents from oocytes expressing human Na_{X} with the indicated proteins co-expressed. FIG. 8C, right panel, show I-V plots with steps between +80 and −100 mV, in 20 mV increments, from a HP of 0 mV. FIG. 8D shows I-V plots with depolarizing steps between −100 and +100 mV, in 20 mV increments, from a HP of −100 mV.

FIG. 9 shows multiple-sequence alignment of Na_{X} and Na_{V} channels. Na_{X} domain boundaries are shown for reference. Na_{X} gating charges are denoted by asterisks and selectivity filter residues are boxed. The Na_{X} DIII-DIV linker IFI-motif is denoted by three consecutive asterisks.

FIGS. 10A-C show β3-Na_{X} channel sample purification. FIG. 10A show β3-Na_{X} expression, purification and reconstitution scheme. FIG. 10B show size-exclusion chromatography elution profile for β3-Na_{X} sample in lipid nanodiscs (MSP1E3D1). FIG. 10C show SDS-PAGE analysis of size exclusion chromatography elution fractions.

FIGS. 11A-L show cryo-EM data processing workflow. FIG. 11A shows representative cryo-EM micrograph of β3-Na_{X}-nanodisc sample. FIG. 11B shows representative 2D class averages. FIG. 11C shows heat map showing the overall distribution of assigned particle orientations in the final reconstruction. FIG. 11D shows global resolution estimate based off the Fourier Shell Correlation (FSC) between two half datasets. FIG. 11E shows isosurface rendering of the final 3D reconstruction colored by local resolution, as estimated by windowed FSCs. FIG. 11F shows cryo-EM map shown over transmembrane regions of DI. FIG. 11G shows representative cryo-EM micrograph of β3-Na_{X}-detergent (GDN) sample. FIG. 11H shows representative 2D class averages. FIG. 11I shows heat map showing the overall distribution of assigned particle orientations in the final reconstruction.

FIG. 11J shows global resolution estimate based off the Fourier Shell Correlation (FSC) between two half datasets. FIG. 11K shows isosurface rendering of the final 3D reconstruction colored by local resolution, as estimated by windowed FSCs. FIG. 11L shows cryo-EM map shown over transmembrane regions of DI.

FIGS. 12A-B show cryo-EM data processing workflows. FIG. 12A shows schematic of the cryo-EM data processing workflow for the β3-Na_{X}-nanodisc sample. FIG. 12B shows schematic of the cryo-EM data processing workflow for the β3-Na_{X}-detergent sample.

FIGS. 13A-F show the overall Na_{X} structure, β3 interactions, and comparison to Na_{V} channels. FIG. 13A shows cryo-EM reconstruction of the β3-Na_{X}-nanodisc complex. The Na_{X} NTD (N-terminal domain) is on the intracellular side and the β3-subunit ECD (extracellular domain) is on the extracellular side. Approximate membrane boundaries are indicated. FIG. 13B shows side-view of the β3-Na_{X} complex. Insets highlight β3 interactions with Na_{X}. FIGS. 13C-E show close-up views of 3-Na_{X} aligned with human Na_{V}1.2 (PDB 6J8E), Na_{V}1.4 (PDB 6AGF), Na_{V}1.5 (PDB 6UZ0) and Na_{V}1.7 (PDB 6J8J) structures from various perspectives. FIG. 13F shows close-up view into the central cavity highlighting the DIV W1484 side-chain of Na_{X} with Na_{V}1.7 superimposed (PDB 6J8J). DIV Phe of Na_{V}1.7 is shown in stick representation.

FIGS. 14A-F show DIII-DI linker, DIII chimeric channels, and lipid bound pore structures of Na_{X}. FIG. 14A shows side- and intracellular view of DIII and DIV from β3-Na_{X}-nanodisc structure with the IFI-motif of the DIII-DIV linker shown in spheres, with equivalent region of Na_{V}1.7 (PDB 6J8J) shown for comparison. FIG. 14B shows multi-sequence alignment of the DIII-DIV linker region with the IFI/IFM-motif. FIG. 14C shows representative currents from oocytes expressing wild-type or IFI>QQQ-mutant human Na_{X} with voltage steps between +80 and −100 mV, in 20 mV increments, from a HP of 0 mV, or depolarizing steps between −100 and +120 mV, in 20 mV increments, from a HP of −100 mV. I-V plots are shown in FIG. 14C, right panel. FIG. 14D shows schematic and representative currents from oocytes expressing various DIII human Na_{V}1.7-Na_{X} channel chimeras in response to voltage steps between +80 and −100 mV, in 20 mV increments, from a HP of 0 mV. FIG. 14D, right panel, maximal current amplitudes at +80 mV (top panel) and −100 mV (bottom panel) of various DIII Na_{V}1.7-Na_{X} chimeras. FIG. 14E shows side-view of the β3-Na_{X}-nanodisc S6-gate structure with gate-lining side-chains shown in stick and semi-transparent surface representation and assigned phosphatidylethanolamine shown in sticks. FIG. 14F shows top-view of the pore comparing the β3-Na_{X}-nanodisc and β3-Na_{X}-detergent-based structures with assigned lipids in the pore shown in stick representations. Cryo-EM map is shown around modeled lipids in mesh representation for the β3-Na_{X}-GDN structure.

FIGS. 15A-C show structure and comparison of the Na_{X} and Na_{V}1.7 selectivity filters. FIG. 15A shows equivalent views of the Na_{X} and Na_{V}1.7 (PDB 6J8J) selectivity filters. Tetrodotoxin bound in Na_{V}1.7 is shown with select interactions. FIG. 15B shows representative currents from Xenopus laevis oocytes expressing wild-type or mutant human Na_{V}1.7 and Na_{X} channel constructs in the presence of 115 mM extracellular Na+, K+, Li+ or Cs+. Steps between −50 and +50 mV, in 10 mV steps, from a HP of −100 mV are shown. FIG. 15C shows representative currents from HEK293 cells expressing human Na_{X}-QTT (containing a C-terminal GFP-Flag tag) in response to a voltage ramp from −80 to +80 mV in the presence (left) or absence (right) of chloride (Cl−) ions (substituted with methanesulfonate, MS−).

FIGS. 16A-E show structure and comparison of the Na_{X} voltage sensor-like domains. FIG. 16A shows equivalent views of Na_{V}1.7 VSD4 (PDB 6J8J) and Na_{X} VSLD4 with gating charge side-chains shown in sticks and R3-R5 labeled for reference. FIG. 16B shows voltage dependence of activation and steady-state inactivation of wild-type Na_{V}1.7 and various mutant channels. Activation, currents were obtained following depolarizing steps between −80 and +65 mV, in 5 mV increments, from a HP of −100 mV. Inactivation, currents were obtained during the test pulse at 0 or −20 mV (25 ms) after a series of conditioning pulses (between −120 and −10 mV, in 5 mV increments, 500 ms) from a HP of −100 mV. Normalized activation and inactivation curves are shown to the right of the current traces. FIG. 16C shows superposition of Na_{X} VSDL3 with the KCNQ1 VSD (PDB 6UZZ) with Na_{X} gating charges and HCS shown as sticks. Na_{X} S3 F1101 and the corresponding S3 serine from KCNQ1 are shown in surface representation; note, this position is also conserved as a serine in all human Na_{V} channels. FIG. 16D shows a view of Na_{X} VSDL4 highlighting the proline side-chains that are unique when compared with human Na_{V} channel VSD4 sequences. FIG. 16E shows voltage dependence of activation and steady-state inactivation of wild-type Na_{V}1.5 and triple-proline VSD4 mutant channel. Activation, currents were obtained following depolarizing steps between −80 and +40 mV, in 5 mV increments, from a HP of −100 mV. Inactivation, currents were obtained during the test pulse at 0 or −20 mV (25 ms) after a series of conditioning pulses (between −120 and −10 mV, in 5 mV increments, 500 ms) from a HP of −100 mV. Normalized activation and inactivation curves are shown to the right of the current traces.

FIGS. 17A-B shows structure and comparison of the Na_{X} selectivity filter and S6-gate regions. FIG. 17A shows a close-up view of the selectivity filer with a putative ion binding site (DEE motif) in Na_{X} shown with a portion of the cryo-EM map (mesh) and a Na+ ion modeled as a sphere. The Na+ ion assigned in the DEE motif binding site in Na_{V}1.2 (PDB 6J8E) and NaVPaS (PDB 6NT4) are shown for comparison. FIG. 17B shows an intracellular view of the Na_{X} S6-gate with Tyr1491 (S6) and bound phosphatidylethanolamine shown and compared to Na_{V}1.4 (assumed to be in a non-conductive, inactivated state; PDB 6AGF) and drug-bound Na_{V}1.5 (assumed to be in a non-conductive, drug blocked state; PDB 6UZ0). Assigned lipid, detergent and drug molecules (PE, GDN, or flecainide) shown as stick representations, respectively.

FIG. 18 shows current amplitudes of Xenopus laevis oocytes expressing the constructs 1-25. (*p<0.05; **p<0.01; ****p<0.0001; one-way ANOVA, Dunnett's test against Na_{X}.)

FIGS. 19A-B show structures of the Na_{X} channel. In FIG. 19A, the DIII+ DIII-IV linker (920-1237) is shown. In FIG. 19B, the S6 gate region is shown.

FIG. 20 shows a multiple-sequence alignment of the Na_{X} channel S6 segment (partial) and the Na_{V} channels.

### DETAILED DESCRIPTION

### I. Definitions

Unless otherwise defined, scientific and technical terms used in connection with the present invention shall have the meanings that are commonly understood by those of ordinary skill in the art. As utilized in accordance with the present disclosure, the following terms, unless otherwise indicated, shall be understood to have the following meanings:

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, the transition term “consisting essentially of,” when referring to steps of a claimed process signifies that the process comprises no additional steps beyond those specified that would materially affect the basic and novel characteristics of the process. As used herein, the transition term “consisting essentially of,” when referring to a composition or product, such as a kit, signifies that it comprises no additional components beyond those specified that would materially affect its basic and novel characteristics.

As used herein, the singular forms “a,” “and,” and “the” include plural referents unless the context clearly dictates otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

All proteins described herein are human proteins unless expressly described otherwise, as in “mammalian Na_{V}” or the like phrases. A “mammalian” protein includes the human protein as well as the equivalent protein from other mammalian species.

An “Na_{V}” protein or “Nav” or “NaV” protein, as described herein, is a member of a voltage-gated sodium channel protein family. Examples of Na_{V} family members in humans and other mammals include Na_{V}1.1, Na_{V}1.2, Na_{V}1.3, Na_{V}1.4, Na_{V}1.5, Na_{V}1.6, Na_{V}1.7, Na_{V}1.8, and Na_{V}1.9. Human Na_{V} family member proteins comprise four domains (DI, DII, DIII, and DIV), each comprising six transmembrane alpha helices (S1, S2, S3, S4, S5, and S6), in some cases with loops or linkers in between certain alpha helices within a domain and between the four domains, for a total of 24 transmembrane alpha helices.

An “Na_{X}” ion channel protein or “Na_{X}” or “NaX” protein, as described herein, is a member of the voltage-gated sodium channel protein family and is also known as Na_{V}2.1 or scn7a. Unless specified otherwise, the “Na_{X}” protein means the “human Na_{X}” protein. The human Na_{X} protein has been reported to act as a sodium sensor in the central nervous system and also in the skin and other epithelia. The amino acid sequence of wild-type human Na_{X} is provided in the UniProt database (Accession No. Q01118), and is provided herein as SEQ ID NO: 1. A human Na_{X} protein also includes naturally occurring variants of the protein, examples of which may also be found in the UniProt database under No. Q01118, for example. Like the human Na_{V} proteins, human Na_{X} comprises four domains (DI, DII, DIII, and DIV), each comprising six transmembrane alpha helices (S1, S2, S3, S4, S5, and S6).

A “mutant human Na_{X}” as used herein refers to a human Na_{X} protein that comprises at least one engineered amino acid substitution, insertion or deletion compared to the wild-type Na_{X} protein of SEQ ID NO: 1. In some cases a “mutant human Na_{X}” comprises at least one amino acid substitution in which at least one residue of wild-type human Na_{X} is substituted with the equivalent/corresponding residue of a mammalian or human Na_{V}. In some cases, a region or a complete domain of wild-type human Na_{X} is substituted for the equivalent region or domain of a mammalian or human Na_{V}, e.g., some or all of DIII and/or the DIII-DIV linker. In such cases, the mutant human Na_{X} may alternatively be referred to herein as a “chimeric human Na_{X}” or a “chimeric Na_{X}” or “chimera,” as it contains amino acid sequence segments from a different protein, a human or mammalian Na_{V}.

The term “chimeric” herein, when referring to a protein, means that the protein is made up of amino acid sequences from more than one native protein. For instance, as described above, a “chimeric human Na_{X}” as used herein refers to a type of mutant human Na_{X} in which at least one region of the Na_{X} protein has been exchanged with a corresponding region from a human Na_{V} family member protein.

The term “chimeric” herein, when referring to a protein, means that the protein is made up of amino acid sequences from more than one native protein. For instance, a “chimeric human Na_{X}” as used herein refers to a type of mutant human Na_{X} in which at least one region of the Na_{X} protein has been exchanged with a corresponding region from a Na_{V} family member protein, such as from a mammalian or human Na_{V} family member protein, such as human or mammalian Na_{V}1.1, Na_{V}1.2, Na_{V}1.3, Na_{V}1.4, Na_{V}1.5, Na_{V}1.6, Na_{V}1.7, Na_{V}1.8, or Na_{V}1.9. In some cases, the exchanged regions may be complete domains, or one of more individual transmembrane alpha helices or linker regions or loops within a domain, or a portion of an alpha helix and/or loop within a domain.

As used herein, the terms “corresponding” or “equivalent” are used interchangeably when referring to a residue or region from a human or mammalian Na_{V} protein that replaces a residue or region deleted from human Na_{X} in making a mutant or chimeric human Na_{X}. As used herein, “corresponding” or “equivalent” residues or regions are those residues or regions that are in the same location within the two proteins when properly folded. In some cases, such corresponding or equivalent regions or amino acid residues may be identified using sequence alignments and structural information for the two proteins.

A “modulator” as used herein refers to a molecule that is capable of altering the behavior of a protein, such as an ion channel protein like human Na_{X}. For example, in some cases a modulator may alter the behavior of a target protein through binding to the protein. A modulator herein may act to either increase or decrease the activity of the protein, such as, for example, the degree to which the protein regulates the flow of ions across a cellular membrane. A modulator that decreases the activity of human Na_{X}, for example, is an “inhibitor” of human Na_{X} activity. A modulator that increases the activity of human Na_{X}, for example, is an “activator” of human Na_{X} activity. In some embodiments, a modulator may only modulate the activity of human Na_{X} under certain conditions, such as in the presence of certain ions or in the presence of certain secondary molecules.

As used herein, a “potential modulator” of human Na_{X} is a molecule that is to be tested to determine if it acts as a modulator of human Na_{X}.

An “ion channel assay” herein refers to an assay that is used to measure the activity of an ion channel protein, such as a voltage-gated sodium channel. A variety of assays and assay formats are regularly used in the art, and many are provided in automated formats for high-throughput screening (HTS) analysis. Examples include ion flux assays, for example, using radioactive ions such as radioactive Na⁺ ions, an ion- or voltage-sensitive dye assay such as fluorescence assays for example using fluorescent indicator molecules whose fluorescence signal increases or decreases with changes in ion concentration, and various types of patch clamp assays. Such assays may directly or indirectly measure changes in ionic currents across a membrane comprising an ion channel protein in a variety of conditions. In some cases, such assays are used to assess the “activity” of the ion channel protein in the presence or absence of a potential or identified modulator. The term “activity” in this sense is meant in the broadest sense, given that these different assays measure the activity of the protein either directly or indirectly and through the measurement of different parameters, such as changes in fluorescence, radioactivity, or ionic current.

A “patch clamp assay” is used herein in the broadest sense to refer to an assay that is used to assesses changes in the movement of ions across a small patch of cell membrane containing an ion channel protein such as Na_{X} or Na_{V} under different solution conditions, for example.

The term “peptide” as used herein refers to a chain of fifty amino acids or less linked by peptide bonds, including amino acid chains of 2 to 50, 2 to 15, 2 to 10, 2 to 8, or 6 to 14 amino acids.

The term “small molecule” as used herein refers to an organic molecule having a molecular weight of 50 Daltons to 2500 Daltons.

The term “macrocycle” or “macrocylic molecule” as used herein refers to a cyclic macromolecule or a macromolecular cyclic portion of a macromolecule. Macrocycles range in size from 500 Daltons to 7500 Daltons. In some cases herein, macrocycles are cyclic peptides or peptide derivatives.

The term “binding fragment” as used herein refers to a portion of a larger molecule, such as a small molecule, peptide, or antibody, that is expected to directly contact a target protein. Binding fragments may be used in high-throughput screens.

In this disclosure, “binds” or “binding” or “specific binding” and similar terms, when referring to a molecule that “binds” to a protein such as a Na_{X} or Na_{V} protein, for example, means that the binding affinity is sufficiently strong that the interaction between the members of the binding pair cannot be due to random molecular associations (i.e. “nonspecific binding”). Thus, the binding is selective or specific.

The term “competition assay” as used herein refers to an assay in which a molecule being tested prevents or inhibits specific binding of a reference molecule to a common target. Other definitions are included in the sections below, as appropriate.

### II. Mutant Na_{X} Proteins

In some embodiments, the invention comprises a mutant human Na_{X} ion channel protein.

Human Na_{X} is a member of the voltage-gated sodium channel protein family and is also known as Na_{V}2.1 or scn7a. Information on the genomic and amino acid sequences of the protein may be found, for example, in the UniProt database under Accession No. Q01118. In some embodiments, the amino acid sequence of human Na_{X}, including the signal sequence, is that of SEQ ID NO: 1. The human Na_{X} protein has been reported to act as a sodium sensor in the central nervous system and also in the skin and other epithelia. The human Na_{X} and Na_{V} proteins comprise four domains (DI, DII, DIII, and DIV), each comprising six transmembrane alpha helices (S1, S2, S3, S4, S5, and S6). Within those domains are further regions, as described in the Table 1 below, with the corresponding amino acid residue positions for each. For example, each domain also comprises a “voltage-sensor domain” or “VSD” (e.g., VSD1, VSD2, VSD3, VSD4) or “voltage-sensor-like domain” or “VSLD” (e.g. VSLD1, VSLD2, VLSD3, VSLD4), comprising the S1-S4 helices and their intervening linkers or loops; specific linkers or loops between the alpha helices, which occur on the extracellular or intracellular face of the protein, and which include, for example, an S4-S5 linker and a pore-forming selectivity filter loop between S5 and S6; and linkers between the four domains. Specific helices, linkers, loops, and other regions of human Na_{X} of SEQ ID NO: 1 are as shown below:

TABLE 1
Human SCN7A, Naₓ or Nav2.1 [UniProtKB - Q01118]

| Topology | | Sequence |
|---|---|---|
| Sequence | | (based on |
| region | Description | cryoEM) |
| 1-118 | Cytoplasmic | |
| 119-137 | Transmembrane, Name = S1 of DI | [118-137] |
| 138-144 | Extracellular | |
| 145-165 | Transmembrane; Name = S2 of DI | [146-165] |
| 166-179 | Cytoplasmic | |
| 180-197 | Transmembrane; Name = S3 of DI | [179-194] |
| 198-203 | Extracellular | |
| 204-220 | Transmembrane; Name = S4 of DI | [204-218] |
| 221-239 | Cytoplasmic; Name = S4-S5 linker of DI | |
| 240-259 | Transmembrane; Name = S5 of DI | [237-258] |
| 260-338 | Extracellular | |
| 339-363 | Pore-forming selectivity filter loop; | |
| | Name = P1 and P2 of DI | |
| 364-370 | Extracellular | |
| 371-391 | Transmembrane; Name = S6 of DI | [371-402] |
| 392-505 | Cytoplasmic; Name = DI-DII linker | [404-495] |
| 506-524 | Transmembrane, Name = S1 of DII | [509-523] |
| 525-535 | Extracellular | |
| 536-555 | Transmembrane; Name = S2 of DII | [531-556] |
| 556-569 | Cytoplasmic | |
| 570-589 | Transmembrane; Name = S3 of DII | [569-583] |
| 590-591 | Extracellular | |
| 592-609 | Transmembrane; Name = S4 of DII | [594-608] |
| 610-625 | Cytoplasmic; Name = S4-S5 linker of DII | |
| 626-644 | Transmembrane; Name = S5 of DII | [626-648] |
| 645-673 | Extracellular | |
| 674-694 | Pore-forming selectivity filter loop; | |
| | Name = P1 and P2 of DII | |
| 695-707 | Extracellular | |
| 708-727 | Transmembrane; Name = S6 of DII | [705-732] |
| 728-935 | Cytoplasmic; Name = DII-DIII linker | [733-920] |
| 936-953 | Transmembrane; Name = S1 of DIII | [934-950] |
| 954-966 | Extracellular | |
| 967-985 | Transmembrane; Name = S2 of DIII | [965-987] |
| 986-999 | Cytoplasmic | |
| 1000-1018 | Transmembrane; Name = S3 of DIII | [1000-1017] |
| 1019-1021 | Extracellular | |
| 1022-1040 | Transmembrane; Name = S4 of DIII | [1026-1039] |
| 1041-1057 | Cytoplasmic; Name = S4-S5 linker of DIII | |
| 1058-1077 | Transmembrane; Name = S5 of DIII | [1059-1078] |
| 1078-1128 | Extracellular | |
| 1129-1150 | Pore-forming selectivity filter loop; | |
| | Name = P1 and P2 of DIII | |
| 1151-1167 | Extracellular | |
| 1168-1189 | Transmembrane; Name = S6 of DIII | [1170-1200] |
| 1190-1252 | Cytoplasmic; Name = DIII-DIV linker | [1201-1237] |
| 1253-1270 | Transmembrane; Name = S1 of DIV | [1251-1269] |
| 1271-1281 | Extracellular | |
| 1282-1300 | Transmembrane; Name = S2 of DIV | [1280-1301] |
| 1301-1312 | Cytoplasmic | |
| 1313-1330 | Transmembrane; Name = S3 of DIV | [1314-1327] |
| 1331-1343 | Extracellular | |
| 1344-1360 | Transmembrane; Name = S4 of DIV | [1344-1357] |
| 1361-1379 | Cytoplasmic; Name = S4-S5 linker of DIV | |
| 1380-1397 | Transmembrane; Name = S5 of DIV | [1378-1400] |
| 1398-1419 | Extracellular | |
| 1420-1442 | Pore-forming selectivity filter loop; | |
| | Name = P1 and P2 of DIV | |
| 1443-1472 | Extracellular | |
| 1473-1495 | Transmembrane; Name = S6 of DIV | [1471-1502] |
| 1496-1682 | Cytoplasmic | [1503-1682] |

The present disclosure includes, for example, a mutant human Na_{X} in which at least one amino acid residue is substituted with at least one corresponding residue of a wild-type mammalian or human Na_{V}. In some cases, a region or a complete domain of wild-type human Na_{X} is substituted for the equivalent region or domain of a mammalian or human Na_{V}, e.g., some or all of DIII or the DIII/DIV linker. In such cases, the mutant human Na_{X} may alternatively be referred to herein as a “chimeric human Na_{X}” or a “chimeric Na_{X}” or “chimera,” as it contains amino acid sequence segments from a different protein, a mammalian or human Na_{V}. In some cases, the substituted residue or region is from mammalian or human Na_{V}1.1, Na_{V}1.2, Na_{V}1.3, Na_{V}1.4, Na_{V}1.5, Na_{V}1.6, Na_{V}1.7, Na_{V}1.8, or Na_{V}1.9. In some cases, the substituted residue or region is from a human Na_{V}, such as from human Na_{V}1.1, Na_{V}1.2, Na_{V}1.3, Na_{V}1.4, Na_{V}1.5, Na_{V}1.6, Na_{V}1.7, Na_{V}1.8, or Na_{V}1.9, for example, from human Na_{V}1.7.

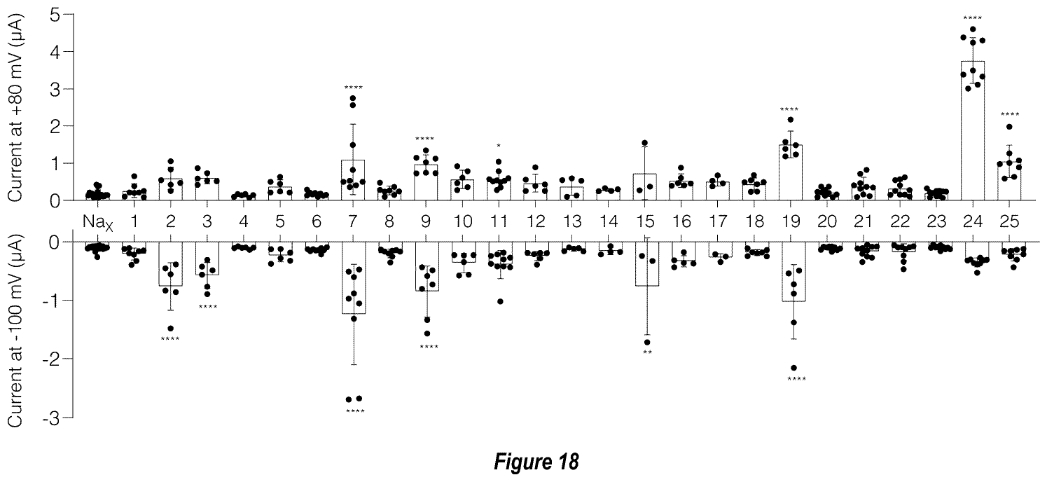

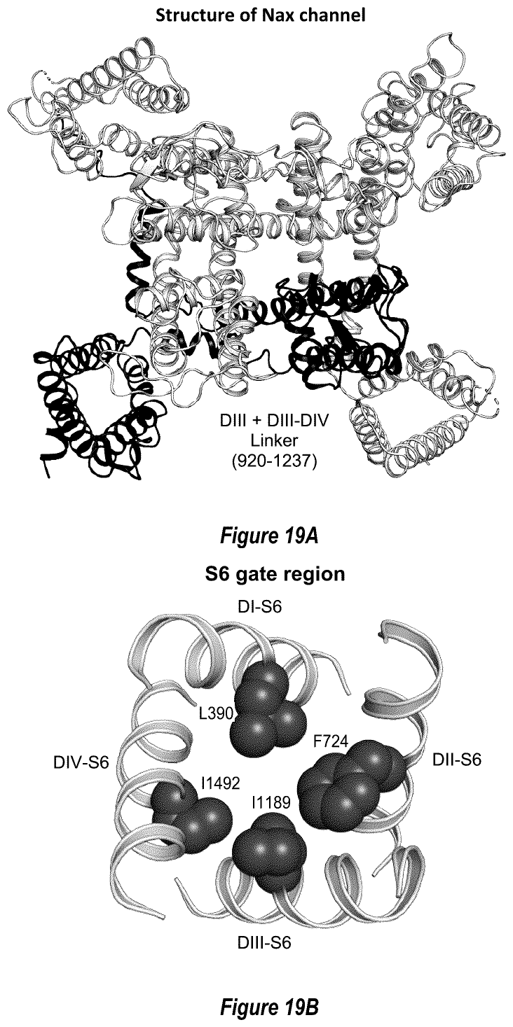

In some embodiments, at least a portion of the DIII domain of human Na_{X} is replaced by a corresponding portion of the DIII domain of a human or mammalian Na_{V} protein (Na_{V}). In some cases, the mutant human Na_{X} comprises all or a portion of the DIII S1-S6 region from a human or mammalian Na_{V}, optionally wherein the at least a portion of the DIII domain of human Na_{X} that is replaced comprises: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the mutant human Na_{X} comprises all or a portion of the DIII S1-S6 region from a human or mammalian Na_{V}, optionally wherein the at least a portion of the DIII domain of human Na_{X} that is replaced consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5. In some cases, the at least a portion of the DIII domain of human Na_{X} that is replaced by a corresponding portion of the DIII domain of the human or mammalian Na_{V} does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. For example, in some cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20. (See Table 4 below and FIG. 18.)

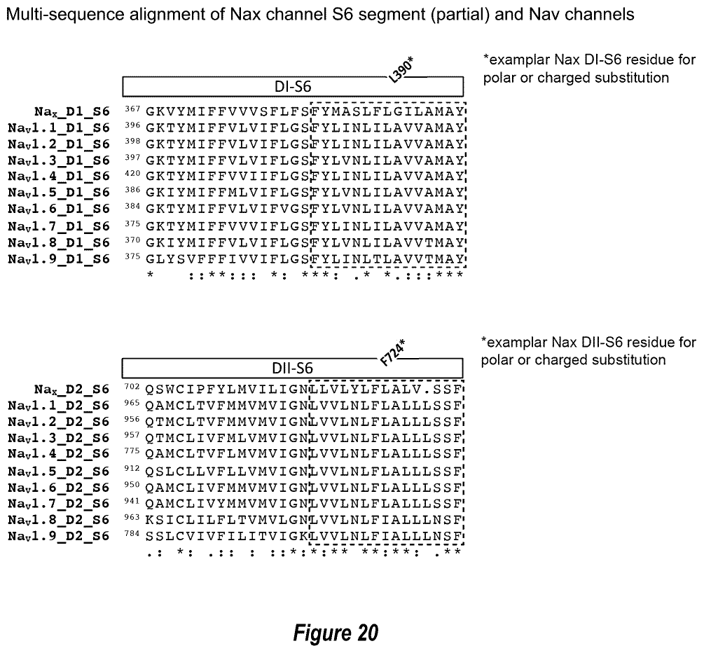

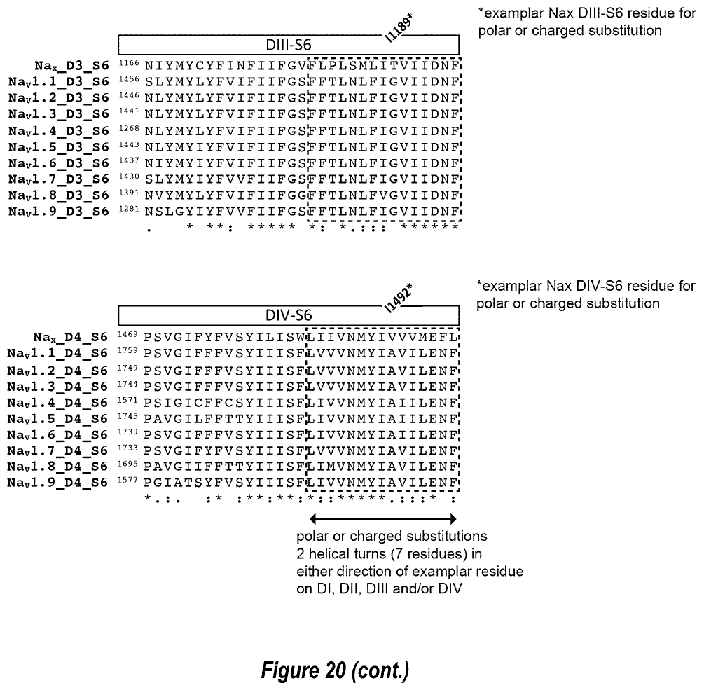

In other cases, a mutant human Na_{X} has at least one substitution of an amino acid residue on one, two, three, or all four of its S6 segments with a polar or charged residue or a glycine or proline residue. In some cases, the substituted residue is polar or charged. Exemplary polar amino acid residues include serine, threonine, tyrosine, asparagine, glutamine, histidine. Exemplary charged amino acid residues include aspartic acid, glutamic acid, lysine, and arginine. These amino acid substitutions can span across the intracellular to the mid-transmembrane region of one, two, three, or all four of the S6 segments (i.e. up to halfway across the membrane bilayer). Predictions of Na_{X} transmembrane segments, for the design of mutations, can be made based on available experimental structures available from the Protein Data Bank (PDB) and standard structure-based multi-sequences alignments with Na_{V} channels (e.g. ClustalW). FIG. 20 depicts the potential location of the amino acid substitutions in human Na_{X}, at S6 of each of DI, DII, DIII, and DIV, as well as their alignment with corresponding residues in other human Na_{V} proteins. As shown by the dashed box on the right side of each sequence, amino acid substitutions may be made at any one of residues L390, F724, I1189, or I1492 or at any residue up to 7 residues in either direction, e.g., at residues 383-397 (in S6 of domain I (D1)), residues 717-731 (in S6 of DII), residues 1182-1196 (in S6 of DIII), and/or residues 1485-1499 (in S6 of DIV). Thus, in some embodiments, at least one of S6 of D1, S6 of DII, S6 of DIII, and S6 of DIV contains at least one substitution of an amino acid residue within the ranges above for a glycine, proline, polar, or charged residue.

In some cases, the mutant human Na_{X} comprises a substitution of at least one residue on one of the four S6 alpha helices with a glycine, proline, or polar or charged residue. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least two of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}. In some cases, the mutant human Na_{X} comprises substitutions of an amino acid residue at all four of residues L390, F724, I1189, and I1492 with a glycine, proline, or polar or charged residue.

In some embodiments, the mutant human Na_{X} protein is active in an ion channel assay. For example, in some cases, an ion channel assay involves expressing the mutant protein in Xenopus laevis oocytes. In some such cases, the current amplitude of Xenopus laevis oocytes expressing the constructs elicited at +80 mV or −100 mV from a holding potential of 0 m V is significantly higher than that from oocytes expressing human Na_{X} wild-type. (See FIG. 18 for illustration of several active and inactive mutant human Na_{X} protein constructs, wherein asterisks denote statistical significance).

### III. Screening Methods Using Mutant Human Na_{X} Proteins

The present disclosure also encompasses, inter alia, methods of identifying molecules that act as modulators of human Na_{X}, comprising screening potential modulators against a mutant human Na_{X}, for example, in an ion channel assay, and/or in a binding assay.

In some embodiments, at least a portion of the DIII domain of human Na_{X} is replaced by a corresponding portion of the DIII domain of a human or mammalian Na_{V} protein (Na_{V}). In some cases, the mutant human Na_{X} comprises all or a portion of the DIII S1-S6 region from a human or mammalian Na_{V}, optionally wherein the at least a portion of the DIII domain of human Na_{X} that is replaced comprises: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the mutant human Na_{X} comprises all or a portion of the DIII S1-S6 region from a human or mammalian Na_{V}, optionally wherein the at least a portion of the DIII domain of human Na_{X} that is replaced consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5. In some cases, the at least a portion of the DIII domain of human Na_{X} that is replaced by a corresponding portion of the DIII domain of the human or mammalian Na_{V} does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a corresponding portion of mammalian Na_{V}1.1, mammalian Na_{V}1.2, mammalian Na_{V}1.3, mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. For example, in some cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20. (See Table 4 below and FIG. 18.)

The present disclosure also encompasses methods of identifying a human Na_{X} ion channel protein (Na_{X}) modulator, comprising: (a) providing a mutant human Na_{X} comprising a substitution of at least one residue on each of two, three, or four S6 alpha helices of human Na_{X} with a glycine, proline, or polar or charged residue; (b) performing an ion channel assay on the mutant human Na_{X} in the presence of the potential modulator; and (c) identifying the potential modulator as a human Na_{X} modulator if the activity of the mutant human Na_{X} in the assay in the presence of the potential modulator is higher or lower than the activity in the absence of the potential modulator. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on one of the four S6 alpha helices with a glycine, proline, or polar or charged residue. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least two of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}. In some cases, the mutant human Na_{X} comprises substitutions of an amino acid residue at all four of residues L390, F724, I1189, and I1492 with a glycine, proline, or polar or charged residue.

The present disclosure further includes methods of determining whether a test molecule that modulates the activity of a human ion channel protein, e.g. a human Na_{V} protein, also modulates the activity of human Na_{X} ion channel (Na_{X}) protein, comprising: (a) providing a mutant human Na_{X} in which at least a portion of the DIII domain of human Na_{X} is replaced by a corresponding portion of the DIII domain of a human or mammalian Na_{V} protein (Na_{V}); (b) performing an ion channel assay on the mutant human Na_{X} in the presence of the test molecule; and (c) determining that the test molecule is a human Na_{X} modulator if the activity of the mutant human Na_{X} in the assay in the presence of the test molecule is higher or lower than the activity in the absence of the test molecule; and optionally, (d) selecting the test molecule for additional screening if it is not a human Na_{X} modulator according to part (c).

The present disclosure additionally includes methods of determining whether a test molecule that modulates the activity of a human ion channel protein, such as a human Na_{V} protein, also modulates the activity of human Na_{X} ion channel (Na_{X}) protein, comprising: (a) determining that the test molecule modulates the activity of the human ion channel protein; (b) providing a mutant human Na_{X} in which at least a portion of the DIII domain of human Na_{X} is replaced by a corresponding portion of the DIII domain of a human or mammalian Na_{V} protein (Na_{V}); (c) performing an ion channel assay on the mutant human Na_{X} in the presence of the test molecule; and (d) determining that the test molecule is a human Na_{X} modulator if the activity of the mutant human Na_{X} in the assay in the presence of the test molecule is higher or lower than the activity in the absence of the test molecule; and optionally, (e) selecting the test molecule for additional screening if it is not a human Na_{X} modulator according to part (d).

In some embodiments, at least a portion of the DIII domain of human Na_{X} is replaced by a corresponding portion of the DIII domain of a human or mammalian Na_{V} protein (Na_{V}). In some cases, the mutant human Na_{X} comprises all or a portion of the DIII S1-S6 region from a human or mammalian Na_{V}, optionally wherein the at least a portion of the DIII domain of human Na_{X} that is replaced comprises: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the mutant human Na_{X} comprises all or a portion of the DIII S1-S6 region from a human or mammalian Na_{V}, optionally wherein the at least a portion of the DIII domain of human Na_{X} that is replaced consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237). In some cases, the at least a portion of the DIII domain of human Na_{X} that is replaced by a corresponding portion of the DIII domain of the human or mammalian Na_{V} does not comprise S5. In some cases, the at least a portion of the DIII domain of human Na_{X} that is replaced by a corresponding portion of the DIII domain of the human or mammalian Na_{V} does not comprise S6. In some cases, the at least a portion of the DIII domain of human Na_{X} that is replaced by a corresponding portion of the DIII domain of the human or mammalian Na_{V} does not comprise any of S1, S2, S3, S4, and/or S4-S5 linker. In some cases, the at least a portion of the DIII domain of human Na_{X} is replaced by a corresponding portion of mammalian Na_{V}1.1, mammalian Na_{V}1.2, mammalian Na_{V}1.3, mammalian Na_{V}1.4, mammalian Na_{V}1.5, mammalian Na_{V}1.6, mammalian Na_{V}1.7, mammalian Na_{V}1.8, mammalian Na_{V}1.9, human Na_{V}1.1, human Na_{V}1.2, human Na_{V}1.3, human Na_{V}1.4, human Na_{V}1.5, human Na_{V}1.6, human Na_{V}1.7, human Na_{V}1.8, or human Na_{V}1.9. For example, in some cases, the at least a portion of the DIII domain of human Na_{X} is replaced by a corresponding portion of human Na_{V}1.7. In some cases, the mutant human Na_{X} is chimera construct 2, chimera construct 3, chimera construct 7, chimera construct 9, chimera construct 11, chimera construct 15 or chimera construct 19, or comprises an amino acid sequence of any one of SEQ ID Nos: 3, 4, 8, 10, 12, 16, or 20. (See Table 4 below and FIG. 18.)

The present disclosure also includes methods of determining whether a test molecule that modulates the activity of a human ion channel protein modulates the activity of human Na_{X} ion channel (Na_{X}) protein, comprising: (a) providing a mutant human Na_{X} comprising a substitution of at least one residue on each of two, three, or four S6 alpha helices of human Na_{X} with a glycine, proline, or polar or charged residue; (b) performing an ion channel assay on the mutant human Na_{X} in the presence of the test molecule; and (c) determining that the test molecule is a human Na_{X} modulator if the activity of the mutant human Na_{X} in the assay in the presence of the test molecule is higher or lower than the activity in the absence of the test molecule; and optionally, (d) selecting the test molecule for additional screening if it is not a human Na_{X} modulator according to part (c).

The disclosure further encompasses methods of determining whether a test molecule that modulates the activity of a human ion channel protein also modulates the activity of human Na_{X} ion channel (Na_{X}) protein, comprising: (a) determining that the test molecule modulates the activity of the human ion channel protein; (b) providing a mutant human Na_{X} comprising a substitution of at least one residue on each of two, three, or four S6 alpha helices of human Na_{X} with a glycine, proline, or polar or charged residue; (c) performing an ion channel assay on the mutant human Na_{X} in the presence of the test molecule; and (d) determining that the test molecule is a human Na_{X} modulator if the activity of the mutant human Na_{X} in the assay in the presence of the test molecule is higher or lower than the activity in the absence of the test molecule; and optionally, (e) selecting the test molecule for additional screening if it is not a human Na_{X} modulator according to part (d).

In some cases, the mutant human Na_{X} comprises a substitution of at least one residue on one of the four S6 alpha helices with a glycine, proline, or polar or charged residue. In some cases, the mutant human Na_{X} comprises a substitution of at least one residue on two of the four S6 alpha helices with a glycine, proline, or polar or charged residue. In some cases, the mutant human Na_{X} comprises a substitution of at least one residue on three of the four S6 alpha helices with a glycine, proline, or polar or charged residue. In some cases, the mutant human Na_{X} comprises a substitution of at least one residue on all four S6 alpha helices with a glycine, proline, or polar or charged residue. In some cases, the mutant human Na_{X} comprises one substitution of an amino acid residue on at least three of the four S6 alpha helices with a glycine, proline, or polar or charged residue. In some cases, the mutant human Na_{X} comprises one or two substitutions of an amino acid residue with a glycine, proline, or polar or charged residue within at least two of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least two of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within at least three of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises one or two substitutions of an amino acid residue with a glycine, proline, or polar or charged residue within each of the following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV). In some cases, the mutant human Na_{X} comprises substitutions of an amino acid residue at two or more of residues L390, F724, I1189, and I1492 with a glycine, proline, or polar or charged residue. In some cases, the mutant human Na_{X} comprises substitutions of an amino acid residue at three of residues L390, F724, I1189, and I1492 with a glycine, proline, or polar or charged residue. In some cases, the mutant human Na_{X} comprises substitutions of amino acid residues L390, F724, and I1189 or of amino acid residues F724, I1189, and I1492 with glycine, proline, or polar or charged residues. In some cases, the mutant human Na_{X} comprises substitutions F724Q, I1189T, and I1492T compared to wild-type human Na_{X}. In some cases, the human Na_{X} comprises substitutions L390E, I1189E, and I1492E compared to wild-type human Na_{X}. In some cases, the mutant human Na_{X} comprises substitutions of an amino acid residue at all four of residues L390, F724, I1189, and I1492 with a glycine, proline, or polar or charged residue.

In certain methods of determining whether a test molecule that is known or discovered to modulate the activity of a human ion channel protein also modulates the activity of human Na_{X} ion channel (Na_{X}) protein, the methods may be used as counter selections. For example, if an experimenter wishes to ensure that a modulator of a particular Na_{V} protein is specific for that Na_{V} protein and/or related Na_{V} proteins, the experimenter may perform the methods herein to confirm that a test molecule is not a modulator of the mutant human Na_{X}.

In any of the above methods, the ion channel assay may be, for example, any suitable assay used to detect the activity of the mutant Na_{X} protein as an ion channel. Examples include, but are not limited to, a patch clamp assay, including an automated patch clamp assay, an ion flux assay, and an ion- or voltage-sensitive dye assay. Exemplary assays are described, for example, in H. Yu et al, “high throughput screening technologies for ion channels,” Acta Pharm. Sinica 37:34-43 (2016), and materials are available from commercial manufacturers. In an ion flux assay, for example, radioactive isotopes, such as sodium 22 (²²Na⁺), can be used to trace the cellular influx and efflux of sodium ions or other ions. Another type of assay is a voltage-sensitive or ion-sensitive dye assay. In a voltage-sensitive dye assay, voltage changes across a membrane comprising the ion channel protein are measured using fluorescence resonance energy transfer (FRET), for example, using dyes such as oxonol derivatives such as bis-(1,3-dibutylbarbituric acid) trimethine oxonol (DiBAC₄) or FMP. In some cases, a FRET dye may be localized or tethered to the membrane. Ion-sensitive dye assays may use dyes that show a difference in signal depending on ion concentration. An example is the sodium indicator dye SBFI. In other embodiments, a patch-clamp assay may be used in the screening methods. In some embodiments, an automated patch-clamp assay may be used. Exemplary platforms and instrumentation for performing patch-clamp assays are sold by several manufacturers. Examples platform assays include IonWorks™ platform assays, PatchXpress™ and IonFlux™ (Molecular Devices), Qpatch™ HT/HTX (Sophion), and Patchliner™ and SynchroPatch™ (Nanion Technologies).

In any of the above methods, the potential modulator identified in the method modulates the activity of the mutant human Na_{X}, e.g., has an activity that is lower or higher than that of wild-type human Na_{X}. In some cases, the potential modulator reduces the activity of the mutant human Na_{X}, such as having an activity that is lower than that of wild-type human Na_{X}. In some cases, the activity may be reduced by at least 25%, at least 30%, at least 40%, at least 50%, at least 60%, at least 70%, at least 75%, at least 80%, or at least 90%. In some cases, the activity the mutant human Na_{X} in the presence of the modulator may be, for example, 1-90%, 10-90%, 1-10%, 1-20%, 25-90%, 50-90%, 25-75%, 40-80%, or 50-75% of the activity of the mutant human Na_{X} in the absence of the modulator. In some cases, the potential modulator identified in the assay reduces the activity of the mutant human Na_{X} in the assay with a half-maximal concentration of 10 nM to 500 M, 50 nM to 500 μM, 10 nM to 50 μM, 100 nM to 500 μM, 100 nM to 50 μM, 1-500 μM, 1-50 μM, 10-500 μM, or 50-250 μM. In other cases, the potential modulator identified in the assay increases the activity of the mutant human Na_{X} in the assay, such as having an activity that is higher than that of wild-type human Na_{X}. In some cases, the activity may be increased by at least 10%, at least 20%, at least 25%, at least 30%, at least 40%, at least 50%, at least 60%, at least 70%, at least 75%, at least 80%, or at least 100% (i.e. two-fold), up to 100%, or up to three-fold. In some cases, the activity the mutant human Na_{X} in the presence of the modulator, may be, for example, 10-100%, 10-80%, 20-80%, 25-100%, 25-75%, 50-100%, two to three fold, or 100-200% higher than the activity of the mutant human Na_{X} in the absence of the modulator.

In any of the above methods, the ion channel assay may also be performed in the presence of both a potential modulator and an identified modulator of the mutant human Na_{X}. For example, in such a case, one might determine if the identified modulator competes with the potential modulator for altering the activity of the protein. In some such cases, the identified modulator of the mutant human Na_{X} is one or more of tetrodotoxin (TTX), quinidine, flecainide, tetracaine, or lidocaine.

Any of the above methods may also further comprise determining the binding affinity of the potential modulator identified in the assay to either or both of the mutant human Na_{X} and wild-type human Na_{X}, and in the case of a chimeric human Na_{X}, to the mammalian or human Na_{V} protein from which the substituted amino acids are derived. For instance, an ELISA assay may be used to determine binding affinity, for example, using mutant human Na_{X} protein in a lipid-stabilized form on a matrix, such as a solid surface, such as beads, plates or the like. Beads can have any shape, such as flakes or chips, spheres, pellets, etc. In some embodiments, such beads are streptavidin-coated beads, avidin-coated beads, or deglycosylated-avidin-coated beads. In some embodiments, such beads are magnetic beads. In some cases, the potential modulator binds to either or both of the mutant human Na_{X} and wild-type human Na_{X} an EC50 or IC50 of 10 μM or less, 10 μM to 50 nM, 10 μM to 500 nM, 1 μM or less, 1 μM to 50 nM, or 100 nM or less.

In some embodiments, further experiments may be performed on molecules selected in the above screens, for example, to determine other biological activities of the molecules. For instance, further assays may be used to determine if the molecules also bind to other ion channel proteins, such as a human or mammalian Na_{V} protein, thus determining the specificity of the molecules as ion channel modulators or binders. For example, in methods using chimeric human Na_{X}, further assays may be performed to determine if the molecules also modulate the activity of the Na_{V} protein from which the chimeric portions of the mutant Na_{X} were derived, or if the molecules bind to that Na_{V} protein.

### IV. Test Molecules for Screening Methods

In any of the above methods, the potential modulator (i.e., the test molecule) may be a peptide or macrocycle or antibody. For example, in some cases, the potential modulator is a small molecule. The present disclosure also relates to modulators of human Na_{X} identified by the methods described herein, which may be optionally peptides, macrocycles, small molecules, or antibodies.

In some embodiments, the potential modulator molecule to be tested is a peptide. In some embodiments, the peptide is a 6-14-mer peptide, such as a 6-12-mer, a 6-10-mer, a 6-8-mer, an 8-12-mer, an 8-10-mer, or the like. See slide 10. In some embodiments, the peptide is a 14-mer. See slides 15-16. In some embodiments, the peptide is an 6-10 mer. In some embodiments, the peptide is an 8-10 mer. In some embodiments, the peptide is an 6-8 mer. In some embodiment, the peptide is an 8-mer. See slides 21-22. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 6-14 mer macrocycle, such as a 6-12-mer, a 6-10-mer, a 6-8-mer, an 8-12-mer, an 8-10-mer, or the like. See slide 10. In some embodiments, the macrocycle is a 14-mer macrocycle. See slides 15-16. In some embodiments, the macrocycle is an 6-10 mer macrocycle. In some embodiments, the macrocycle is an 8-10 mer macrocycle. In some embodiments, the macrocycle is an 6-8 mer macrocycle. In some embodiments, the macrocycle is an 8-mer macrocycle. See slides 21-22. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle. In some embodiments, the macrocycle has at least one lipophilic side-chain and at least one positively charged side-chain.

In some embodiments, the molecule to be tested in a screen herein is a small molecule. In some embodiments, the molecule to be tested is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule to be tested is a binding fragment of a peptide, a binding fragment of a small molecule, or a binding fragment of an antibody (e.g., an antigen binding fragment).

### V. Molecular Complexes

In some embodiments, the disclosure comprises a molecular complex comprising a mutant human Na_{X} as described herein bound to a molecule, such as a potential modulator molecule, such as a peptide, small molecule, antibody, or binding fragment of a peptide, small molecule, or antibody.

In some embodiments, the molecule is a peptide. In some embodiments, the peptide is a 6-14 mer peptide, such as a 6-12-mer, a 6-10-mer, a 6-8-mer, an 8-12-mer, an 8-10-mer, or the like. See slide 10. In some embodiments, the peptide is a 14-mer. See slides 15-16. In some embodiments, the peptide is an 6-10 mer. In some embodiments, the peptide is an 8-10 mer. In some embodiments, the peptide is an 6-8 mer. In some embodiment, the peptide is an 8-mer. See slides 21-22. In some embodiments, the peptide is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer.

In some embodiments, the peptide is a macrocycle. In some embodiments, the macrocycle is a 6-14 mer macrocycle, such as a 6-12-mer, a 6-10-mer, a 6-8-mer, an 8-12-mer, an 8-10-mer, or the like. See slide 10. In some embodiments, the macrocycle is a 14-mer macrocycle. See slides 15-16. In some embodiments, the macrocycle is an 6-10 mer macrocycle. In some embodiments, the macrocycle is an 8-10 mer macrocycle. In some embodiments, the macrocycle is an 6-8 mer macrocycle. In some embodiments, the macrocycle is an 8-mer macrocycle. See slides 21-22. In some embodiments, the macrocycle is a 3-40-mer, a 3-20-mer, a 4-16-mer, a 4-14-mer, or a 6-14-mer, such as a 3-mer, 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer macrocycle. In some embodiments, the macrocycle has at least one lipophilic side-chain and at least one positively charged side-chain.

In some embodiments, the molecule in the complex is a small molecule. In some embodiments, the molecule is an antibody, which may include not only full length antibodies of any of IgG, IgM, IgA, IgD, and IgE, but also an antigen binding fragment of an antibody, such as an Fv, Fab′, (Fab′)2, scFv, or the like, a nanobody, single-chain antibody, bispecific or multispecific antibody.

In some embodiments, the molecule is a binding fragment of a peptide, a binding fragment of a small molecule, or a binding fragment of an antibody (e.g., an antigen binding fragment).

### VII. Kits

The present disclosure also includes kits comprising reagents associated with screening methods herein. In some cases, kits comprise one or more species of mutant human Na_{X} as described herein. In some cases, kits comprise reagents used in screening methods herein, either with or without a particular mutant human Na_{X}. In some cases, kits comprise more than one type of mutant human Na_{X}. In some cases, the kits also comprise at least one mammalian or human Na_{V} and/or wild type human Na_{X}, for example, as a control.

In some embodiments, kits herein may comprise one or more reagents for performing an ion channel assay. In some cases, kits may comprise particular modulators of the mutant human Na_{X}, for instance, as positive controls. Kits herein may also include negative controls that are identified as not modulating the mutant human Na_{X}. Where kits are used to determine whether a molecule binds to the mutant protein, the kits may include mutant human Na_{X} attached to matrix particles such as beads or to another type of matrix such as a plate. Beads can have any shape, such as flakes or chips, spheres, pellets, etc. In some embodiments, such beads are streptavidin-coated beads, avidin-coated beads, or deglycosylated-avidin-coated beads. In some embodiments, such beads are magnetic beads. The mutant human Na_{X} may or may not be pre-attached to a matrix. In some embodiments, reagents are included to facilitate attachment of the protein to a matrix, such as through biotin-streptavidin or a similar system.

In some embodiments, kits may comprise reagents associated with screening methods herein, such as some or all of the reagents needed to perform an ion channel assay as described in the methods herein. In some cases, one or more control reagents, such as modulators of the protein, may also be included.

In some embodiments, kits may comprise test molecules or libraries of test molecules, such as peptides, small molecules, and/or antibodies. In some embodiments, peptides in the kit may be macrocycles. In some embodiments, the kit may comprise test molecules that are a binding fragment of a peptide, small molecule, or antibody.

In some embodiments, kits may also comprise directions for use.

### EXAMPLES

The following are examples of methods and compositions of the disclosure. It is understood that these Examples are not meant to limit the disclosure, but only to exemplify it, and that various other embodiments may be practiced, given the general description provided above.

### Example 1—Materials and Methods

### Example 1.1—Experimental Model and Subject Details

The cell line Expi293F is a human embryonic kidney cell line (female) transformed and adopted to grow in suspension (Thermo Fisher Scientific) and was used for protein expression. Expi293F cells were cultured in SMM 293T-I medium at 37° C. and 5% CO2. Expi293F cells were authenticated and tested for Mycoplasma contamination.

### Example 1.2-Protein Expression and Purification

Full-length human Na_{X} containing tandem 2× StrepII and 2×FLAG tags at its N-terminus and untagged full-length human β3 were each cloned into pRK vectors under the control of a CMV promotor. Both constructs were transfected with PEI into Expi293 cells and cultured for 48 hours in SMM 293T-I medium under 5% CO2 at 37° C. Cells were harvested by centrifugation at 800 g for 10 minutes and resuspended and lysed in Buffer A (25 mM ADA pH 6.0, 200 mM NaCl, 1 mM PMSF, 1 μg/mL benzonase, and 1x Roche protease inhibitor cocktail) by dounce homogenization. To solubilize the cell membranes, glyco-diosgenin (GDN) and cholesterol hemisuccinate (CHS) were added to the sample to final concentrations of 2% and 0.3%, respectively, and the sample was gently stirred at 4° C. for two hours. Insoluble material was then collected by ultracentrifugation at 125,000 g for 1 hour at 4° C. Supernatants were applied to anti-FLAG M2 agarose resin that had been pre-equilibrated in Buffer B (25 mM ADA pH 6.0, 200 mM NaCl, 0.01% GDN) and bound in batch at 4° C. for 1 hour. The sample was then applied to a gravity column and the collected resin was washed with 10 CV Buffer B followed by 10 CV supplemented with 5 mM ATP and 10 mM MgCl₂. Protein was eluted with 5 CV Buffer B supplemented with 300 μg/mL FLAG peptide. Eluates were applied directly to Strep-Tactin XT Superflow high capacity resin that had been pre-equilibrated in Buffer B and bound in batch for three hours, then washed with 10 CV Buffer B prior to sample elution in 5 CV supplemented with 50 mM biotin. For nanodisc incorporation, the sample was concentrated to 15 μM using an Amicon Ultra centrifugal filter device (100 kDa MWCO). For structural analysis in detergent (GDN), the eluate was instead concentrated to 100 μL and applied to a Superose 6 3.2/300 column that had been pre-equilibrated in Buffer B.

### Example 1.3—Mass Spectrometry Analysis

Following size exclusion chromatography, 10 μg of the Na_{X}-containing peak fraction (after co-expression with β1- or β3-subunits, respectively) was denatured with 8 M guanidine (1:1 v/v), reduced with 1M dithiothreitol (DTT, Sigma-Aldrich, St Louis, MO) to a final concentration of 100 mM DTT and incubated at 95° C. for 10 minutes, then centrifuged. 10 μL of 1 M Tris, pH 8 was added to the solution and water was used to dilute the guanidine to a 2 M final concentration. Deglycosylation was performed with 2 μL of PNGaseF (15000 U, New England Biolabs) followed by overnight incubation at 37° C. Samples were further reduced with 10 mM DTT at 60° C. (15 minutes) followed by alkylation with 20 mM iodoacetamide at room temperature. Proteins were digested with 0.2 μg trypsin (Promega) or chymotrypsin in 50 mM ammonium bicarbonate, pH 8 at 37° C. overnight. Digestions were quenched with formic acid and the supernatants were subjected to desalting on C18 PhyTips (PhyNexus), lyophilized, reconstituted in 0.1% formic acid containing 2% acetonitrile and analyzed without further processing by reversed phase nano-LC/MS/MS on a Waters NanoAcquity HPLC system (Waters Corp.) interfaced to an Elite Orbitrap mass spectrometer (ThermoFisher Scientific). Peptides were loaded onto a Symmetry C18 column (1.7 mm BEH-130, 0.1×100 mm, Waters) and separated with a 60 minute gradient from 2% to 25% solvent B (0.1% formic acid, 98% acetonitrile) at 1 μL/min flow rate. Peptides were eluted directly into the mass spectrometer with a spray voltage of 1.2 kV. Full MS data were acquired in FT for 350-1250 m/z with a 60,000-resolution. The most abundant ions from full MS scans were selected for MS/MS through a 2-Da isolation window.

Acquired tandem MS spectra were searched using the Mascot algorithm (Matrix Sciences) with trypsin or chymotrypsin enzyme specificity. Search criteria included a full MS tolerance of 50 ppm, MS/MS tolerance of 0.5 Da with oxidation of methionine (+15.9949 Da) as variable modification and carbamidomethylation (+57.0215 Da) of cysteine as static modification. Data were searched against the specific sequences of in-house constructs overlaid onto the Uniprot mammalian database, including reverse protein sequences. Peptide assignments were first filtered to a 2% false discovery rate (FDR) at the peptide level and subsequently to a 2% FDR at the protein level.

### Example 1.4—Reconstitution of β3-Na_{X} into Lipid Nanodiscs

For reconstitution into nanodiscs, multiple 50 μL sample aliquots were each applied to 2 mL tubes with a 200-fold molar excess of a POPC:POPE:POPG lipid mix (3:1:1 ratio solubilized in a sonication bath at 10 mg/mL in a buffer containing 50 mM HEPES pH 7.5, 100 mM NaCl, 5 mM MgCl₂, and 1% CHAPS) and incubated for 30 minutes at 4° C. The membrane scaffold protein MSP1E3D1 (Sigma) was then applied to the samples in a 4-fold molar excess and incubated for an additional 30 minutes at 4° C. Samples were then diluted to 1.5 mL and Bio-Beads were applied to a final concentration of 0.25 mg/mL. Samples were then mutated overnight at 4° C. prior to Bio-Bead removal. The samples were then pooled and passed over 1 mL Strep-Tactin XT resin in a column that had been pre-equilibrated in Buffer C (25 mM HEPES pH 7.0, 200 mM NaCl) to remove empty nanodiscs. The column was washed with 5 CV Buffer C prior to sample elution with 5 CV containing 50 mM biotin. The sample was then concentrated to 100 μL and applied to a Superose 6 3.2/300 column that had been pre-equilibrated in Buffer C. Peak fractions were pooled and concentrated to 3 mg/mL.

### Example 1.5—Cryo-EM Sample Preparation and Data Acquisition

Samples were cross-linked with a final concentration of 0.05% EM-grade glutaraldehyde at room temperature for 10 minutes. The cross-linking reactions were quenched by the addition of 1 M Tris pH 7.0. Ultrafoil R2/2 (200 mesh) cryoEM grids (Quantifoil GMBH) were plasma cleaned for 5 seconds using the Solarus plasma cleaner (Gatan, Pleasanton, CA). Three microliters of the final samples at 2.25 mg/mL (nanodiscs) or 1.5 mg/mL (GDN) were applied to the cryoEM grids and blotted for 2.5 seconds using a Vitribot Mark IV (ThermoFIsher Scientific, Waltham, MA) using a blot force setting of 8 at 100% humidity, and plunged into liquid ethane. Grids were then imaged on a Titan Krios electron microscope (ThermoFisher Scientific, Waltham, MA) operated at 300 keV with a bioquantum energy filter using a K2 (nanodiscs) or K3 (GDN) Summit direct electron detector (Gatan, Pleasanton, CA). Images of the nanodisc were recorded at a magnification of 165,000x, which corresponded to 0.824 Å/pixel using a 20 eV energy slit. Image stacks contained 50 images recorded at 0.2 second intervals over 10 seconds, giving a total exposure of ˜50 e/Å². Images of the GDN sample were recorded in super resolution mode at 105,000× magnification, corresponding to 0.419 Å/pixel, using a 20 eV energy slit. Image stacks contained 60 images recorded at 0.05 second intervals over 3 seconds, giving a total exposure of ˜64 e/Å². All data collection was done using serialEM⁶⁷.

### Example 1.6—Cryo-EM Data Processing

### Nanodisc Sample

Cryo-EM data was processed using a combination of the WARP, RELION, and cisTEM software packages⁶⁸⁻⁷¹. For the first dataset, 11,658 movies were corrected for frame movement using MotionCor2⁷² in RELION. The resulting images were filtered to retain only those with an accumulated motion total below a value of 250 and contrast-transfer function (CTF) parameters were fit using the 30-4.5 Å band of the spectrum using CTFFIND4.1⁷³. Images were filtered to include only those with a detected fit resolution better than 5 Å, giving a total of 9,988 good images for further processing. 1,669,107 particles were picked using a deep learning-based algorithm in WARP⁶⁸. Particles were subjected to two rounds of 2D classification in cisTEM, and the best 30 classes were chosen (76,392 particles) and exported to RELION for 3D classification. A 20 Å low-pass filtered (LPF) Kv1.2 reconstruction solved in nanodiscs (EMD-9024)⁷⁴ for which all density outside of the nanodisc had been erased was used as the initial 3D reference. The best obtained 3D volume was then used as the reference for a second round of 3D classification using a broader selection of particles from the 2D classification in cisTEM (350,901 particles). The best 3D volume and its corresponding 91,435 particles were then imported back into cisTEM for iterative rounds of auto-refine without a mask and manual refinement with iteratively adjusted masks and 20 Å LPF outside the mask (outside weight of 0.8). The resulting 4.5 Å map was then used as the reference in a final round of unmasked auto-refinements and masked manual refinements in cisTEM using the particle stack from only a single round of 2D classification in cisTEM, giving the final 3.2 Å map.

### Detergent (GDN) Sample

Cryo-EM data was processed using a combination of the RELION, Gautomatch [K. Zhang, MRC LMB (mrc-lmb.cam.ac.uk/kzhang/)], and cisTEM software packages. 10,850 movies were corrected for frame movement using MotionCor272 in RELION and binned to 1 Å/pixel. The resulting images were filtered to retain only those with an accumulated motion total below a value of 250 and contrast-transfer function (CTF) parameters were fit using the 30-4.5 Å band of the spectrum using CTFFIND4.1⁷³. Images were filtered to include only those with a detected fit resolution better than 5 Å, giving a total of 10,569 good images for further processing. A total of 2,780,663 particles were automatically picked using Gautomatch using 16 uniform projections of the reconstruction of Na_{V}1.4 in complex with the β1 auxiliary subunit (EMD-9617) as templates. Particles were subjected to a single round of 2D classification into 250 classes in RELION and a particle stack containing 1,420,422 particles from the best 55 classes were extracted and exported to cisTEM. The structure of β3-Na_{X} solved in nanodiscs was used as an initial reference in an initial round of auto-refine without a mask, followed by several rounds of manual refinement with iterative mask refinement and 20 Å LPF outside the mask (outside weight of 0.8). Manual refinement runs included defocus refinement and used a score threshold of 0.2. Iterative rounds of unmasked auto-refinements and masked manual refinements yielded a final reconstruction of 2.85 Å resolution.

### Example 1.7—Model Building and Structure Analysis

The structure of Na_{V}1.4 in complex with the β1 auxiliary subunit (PDB 6AGF) 28 was used as a template to generate a Na_{X} homology model using the Phyre2 server⁵⁸. This model along with β1 was rigid body docked into the cryo-EM map for manual rebuilding in Coot⁵⁹ to give an initial model of the β3-Na_{X} complex. Multiple rounds of real space refinement in Phenix⁶⁰ and manual rebuilding in Coot were followed by molecular dynamics-assisted manual refinement in UCSF ChimeraX⁶¹ with ISOLDE⁶². After a final refinement in Phenix, the model was validated using the Phenix validation package. Structure figures were generated using PyMol⁶³, UCSF Chimera⁶⁴, and UCSF ChimeraX. Caver3.0 was used to analyze the channel pore⁶⁵. Sequence alignments were performed using Clustal Omega⁶⁶ and rendered with ESPript 3.0⁶⁷.

### Example 1.8—Molecular Biology for Biochemical and Electrophysiological Experiments

Complementary DNAs (cDNAs) of human Na_{X}, Na_{X}-eGFP-2×FLAG, Na_{V}1.7, Na_{X}-Na_{V}1.7 domain-swapped chimeras, ATP1A1, ATP1B1 and SAP97, codon optimized for Homo sapiens, were cloned into the pcDNA3.1/Hygro (+) vector were used for this study. The human Na_{V}β1, Na_{V}β3, Ca_{V}β1, Ca_{V}γ2, Ca_{V}α281, ATP1A1, ATP1B1, ATP1G1 and SAP97 constructs were cloned into the pcDNA3.1 (+) vector. Na_{X}, Na_{V}1.5 and Na_{V}1.7 mutants (Na_{X} N1142K, QTT (F724Q/I1189T/I1492T); Na_{V}1.5 S1610P/S1618P/A1640P; Na_{V}1.7 S211P, AL966, S1269F, K1406N and A1596P/S1605P/A1627P) were generated using site-directed mutagenesis with custom-designed primers (Eurofins Genomics), PfuUltra II Fusion HS DNA Polymerase (Agilent Technologies) and DNA sequences were verified by Sanger DNA sequencing (Eurofins Genomics). For expression in Xenopus laevis oocytes, cDNAs were linearized using either NotI, BamHI or XbaI restriction enzyme and then transcribed to capped RNAs with the T7 mMessage mMachine Kit (Ambion). For expression in HEK293T and Neuro-2a cells, plasmid DNAs purified with the NucleoBond Xtra Midi Plus kit (Macherey-Nagel) were used.

### Example 1.9—Two-Electrode Voltage-Clamp Electrophysiology

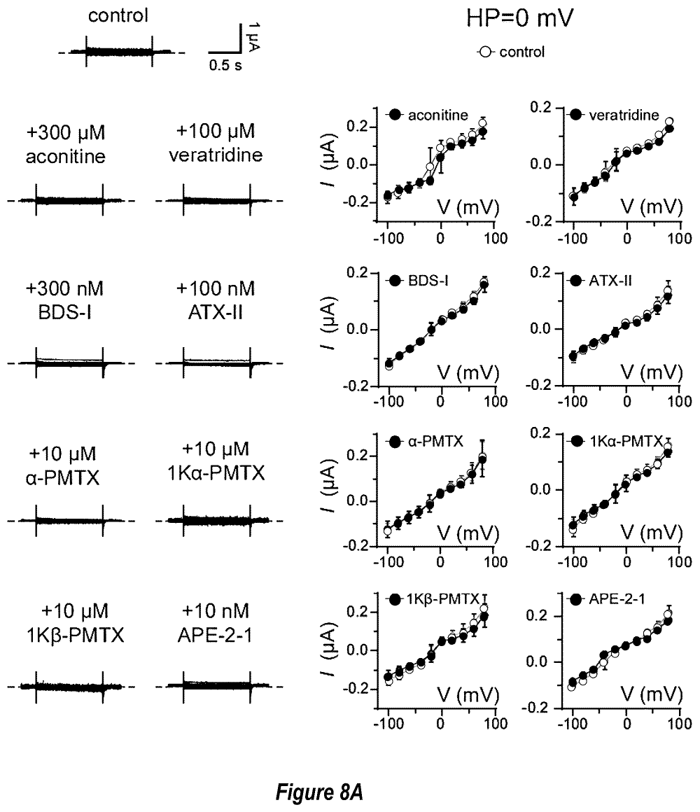

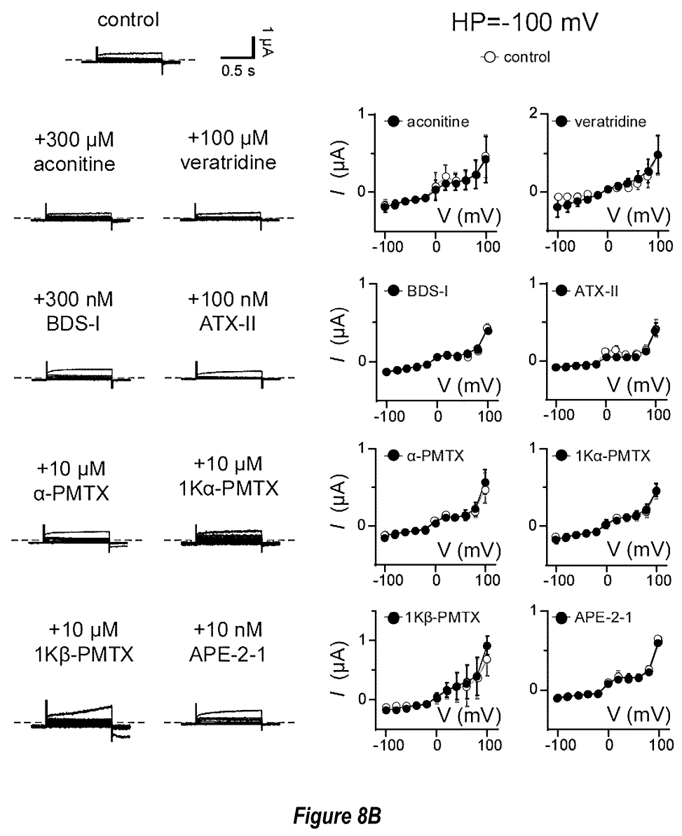

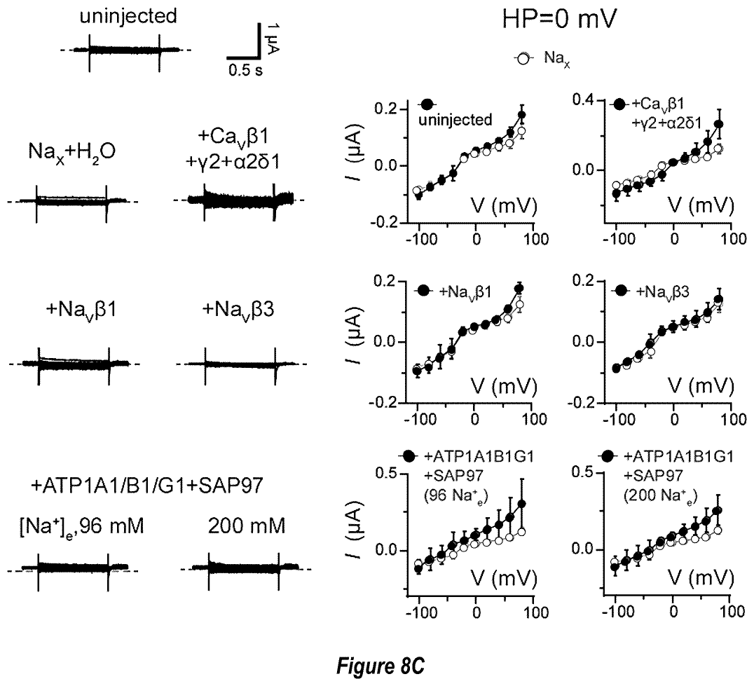

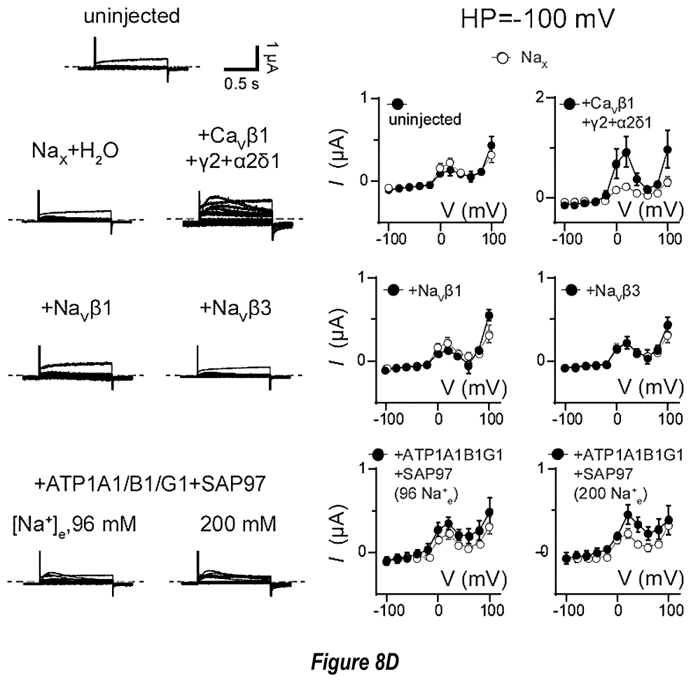

Oocytes were prepared as previously described⁶⁸. Healthy-looking stage V-VI oocytes were injected with 2.5-40 ng of RNA (in 5-41 nL) using a Nanoliter 2010 injector (World Precision Instruments). When excluding one or more construct, an equivalent volume of nuclease-free water was added to maintain a constant RNA concentration across different construct combinations. Injected oocytes were kept at 18° C., 140 rpm, in ND96 supplemented with 50 μg/mL gentamicin and 50 μg/mL tetracycline (in mM: 96 NaCl, 2 KCl, 1 MgCl₂, 1.8 CaCl₂), 2.5 sodium pyruvate, 0.5 theophylline, 5 HEPES; pH 7.4 with NaOH) for two to five days. Two-electrode voltage-clamp measurements were performed at room temperature using a Warner OC-725C Oocyte Clamp amplifier (Warner Instrument Corp, USA) and under constant perfusion with ND96 solution (in mM: 96 NaCl, 2 KCl, 1 MgCl₂, 1.8 CaCl₂, 5 HEPES; pH 7.4 with NaOH). For ion selectivity experiments, external solutions contained 115 mM of test cations as chloride salts, 1.2 mM CaCl₂, 2 mM MgCl₂, 5 mM HEPES (pH 7.4 with the corresponding hydroxide). Data acquisition was performed using a Digidata 1550 digitizer (Molecular devices; sampled at 10 kHz) and pCLAMP 10 software (Molecular Devices). Microelectrodes from borosilicate glass capillaries (Harvard Apparatus) were prepared to have resistances around 0.2-1.0 MQ2 using a P-1000 Flaming/Brown Micropipette Puller System (Sutter Instrument) and backfilled with 3 M KCl. Aconitine and veratridine (Alomone Labs) were dissolved in DMSO to make 50 mM stocks, and were further diluted to 300 and 100 μM in ND96 for electrophysiological experiments. Other Na_{V} activators (Alomone Labs; FIGS. 8A-D) were dissolved in water to make stock solutions. The voltage dependence of ion current was determined using a protocol consisting of steps from a holding potential of 0 mV to voltages ranging from +80 to −100 mV for 1 s in 20 mV increment.

### Example 1.10—Cell Culture, Cell Surface Biotinylation and Western Blots

HEK293T and Neuro-2a cells (ATCC) were grown and maintained as described previously⁶⁸. Approximately 800,000 HEK293T and 250,000 Neuro-2a cells were seeded in 35 mm cell culture dishes ˜20 hours before transient transfection with 1 μg of cDNA using LipoD293 ver. II (tebu-bio). For biochemical experiments, the cDNAs of Na_{X}-eGFP-2×FLAG and β3 were mixed in a mass ratio of 1:1. Equal amount of empty vector DNA was added to keep the total cDNA amount constant when β3 was excluded from transfection. Transfected HEK293T cells were used for biochemical experiments 24 hours post-transfection. To induce differentiation, Neuro-2a cells were serum starved 24 hours post-transfection (cultured in DMEM for additional 24-30 hours) before they were used for biochemical and electrophysiological experiments. Cell surface biotinylation and Western blots were performed as described previously⁶⁸, with only slight modifications: 1) The quenching step was performed under gentle agitation on ice for 30 min, 2) Xenopus laevis oocytes were washed twice with tris-buffered saline before lysis, and 3) the total lysate fraction for oocytes was diluted 1:5 in SDS sample buffer before loading onto the gel due to excess protein. Antibodies were used as stated in⁶⁸, except the mouse anti-Na⁺/K⁺-ATPase antibody was from Santa Cruz Biotechnology (sc-21712). Blots are representative of minimum three individual experiments.

### Example 1.11—Patch Clamp Electrophysiology

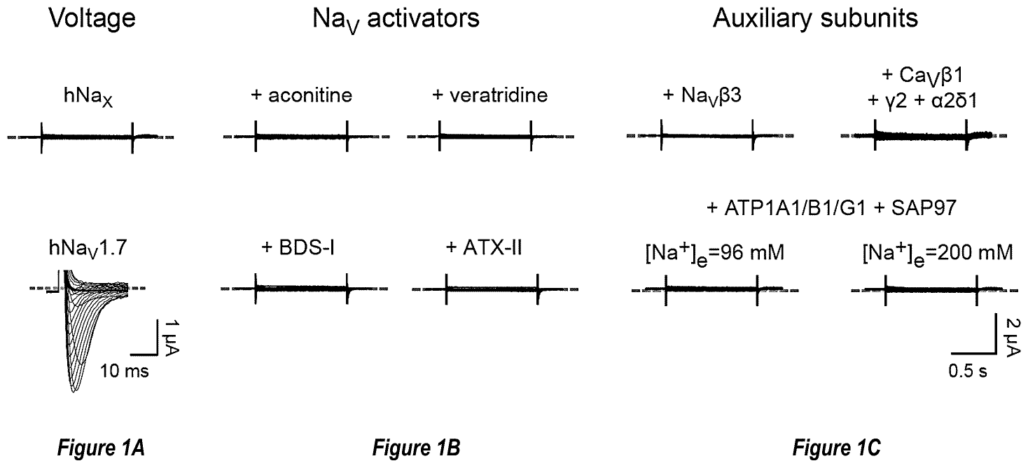

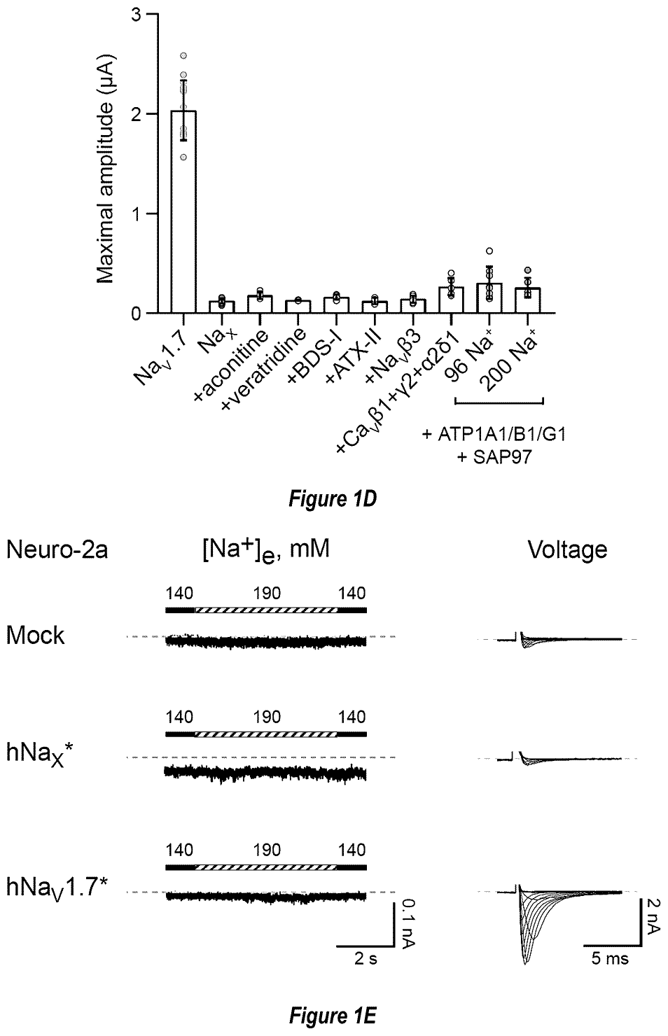

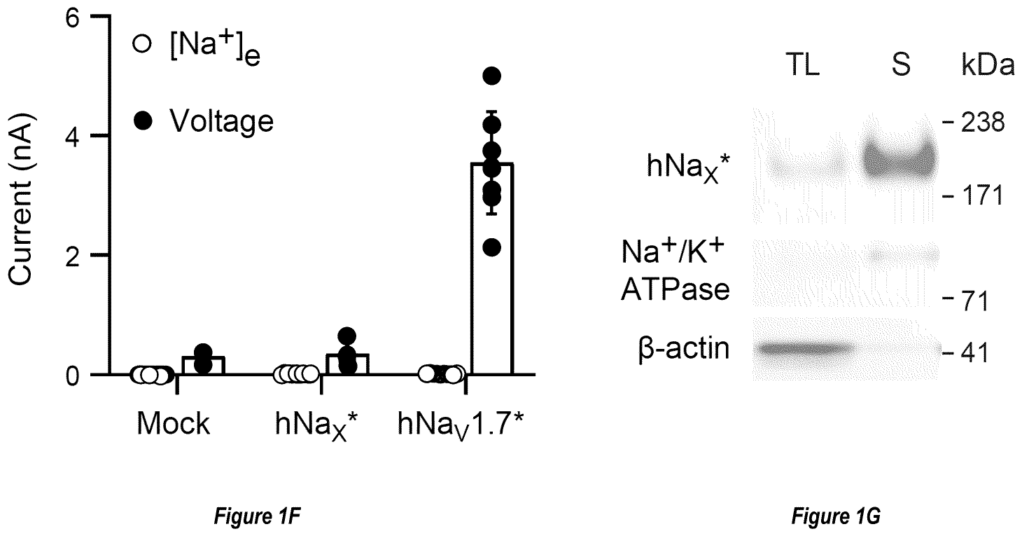

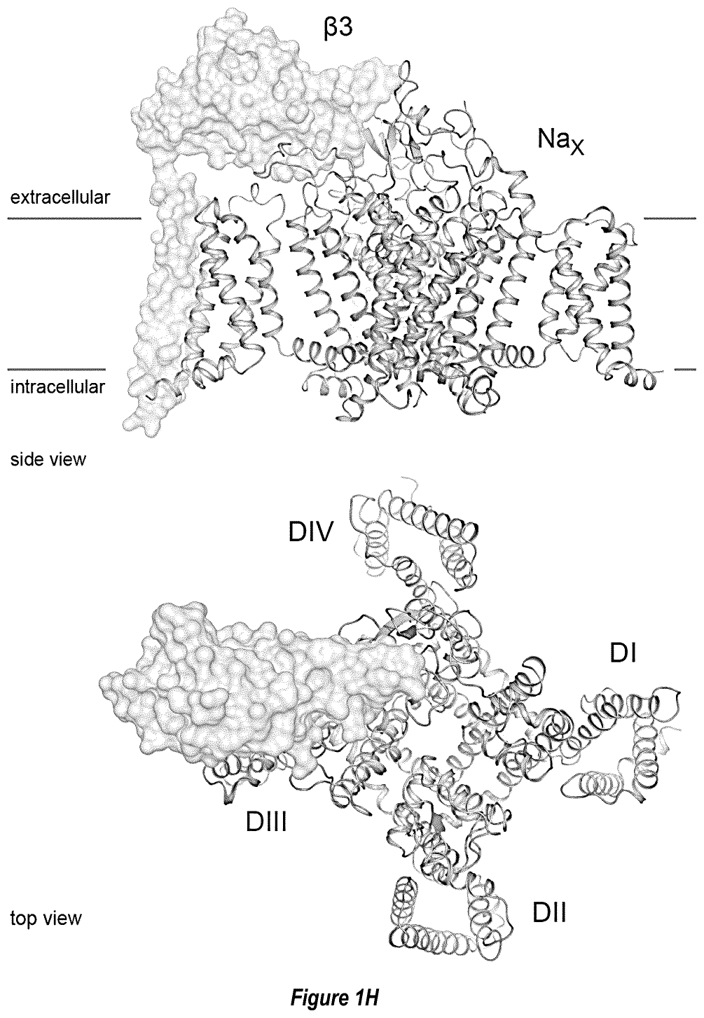

Differentiated Neuro-2a cells expressing Na_{X}-eGFP-2×FLAG were seeded on glass cover slips an hour before patch clamping experiments. Cells were voltage-clamped at −60 m V in whole-cell configuration (FIGS. 1E-F and 7F) using an Axopatch 200B amplifier (Molecular Devices). Digidata 1550A digitizer (Molecular devices; sampled at 10 kHz) and pCLAMP 10 software (Molecular Devices). Glass pipettes for patch-clamp experiments were pulled from borosilicate glass capillaries (World Precision Instruments; Kwik-Fil 1.5/1.12; OD/ID), and fire polished to final resistances between 2.0-5.5 MΩ. Extracellular solutions contained (in mM): 140 or 190 NaCl, 5 KCl, 2.5 CaCl₂), 1 MgCl₂, 5 HEPES, 20 glucose (pH 7.4 with NaOH). The osmolarity values of these solutions were ˜302 mOsm/L (for 140 NaCl) and ˜395 mOsm/L (for 190 NaCl). The intracellular solution contained (in mM): 120 K-gluconate, 20 TEA-C1, 2 MgCl₂, 2Na₂ATP, 1 EGTA and 10 HEPES, pH 7.3 with KOH (˜281 mOsm/L).

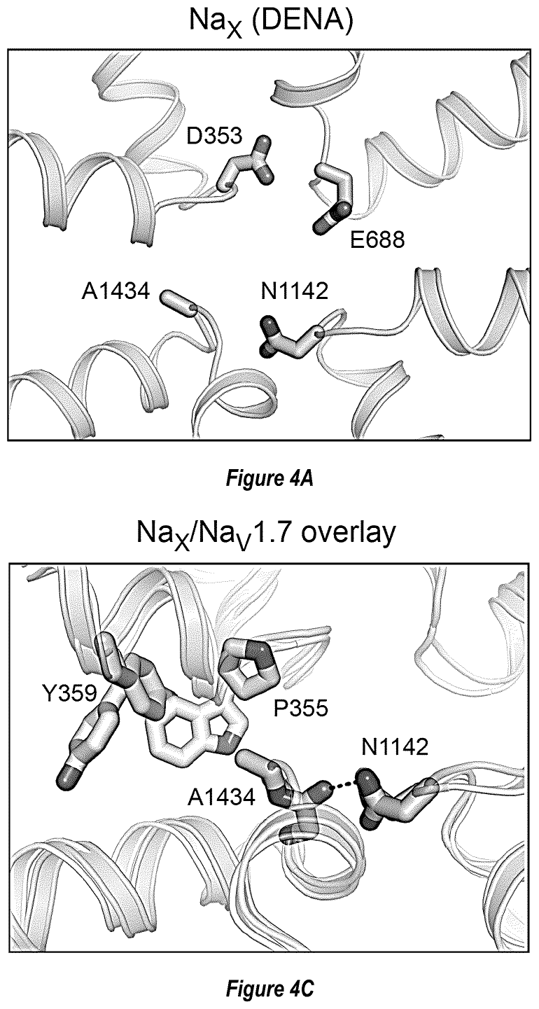

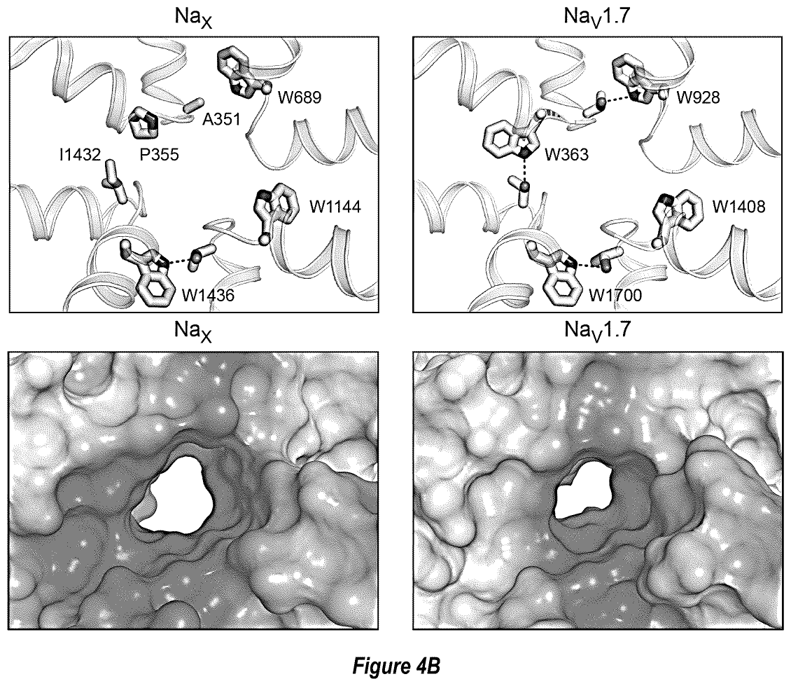

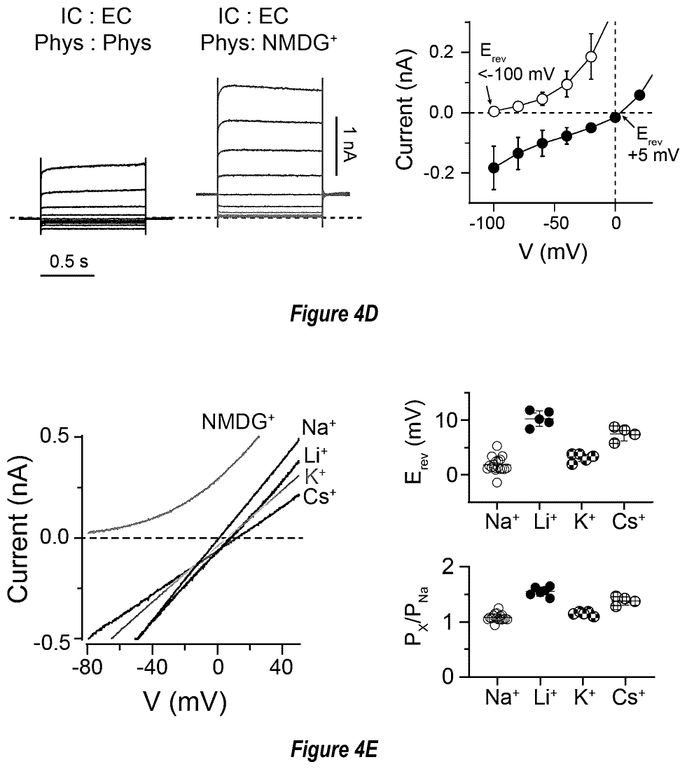

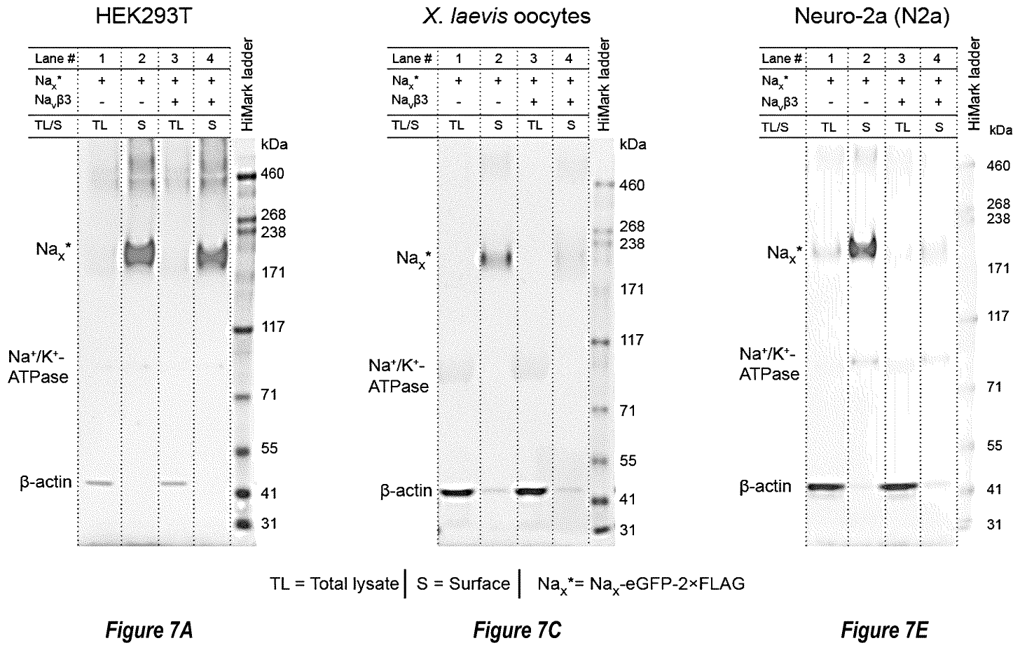

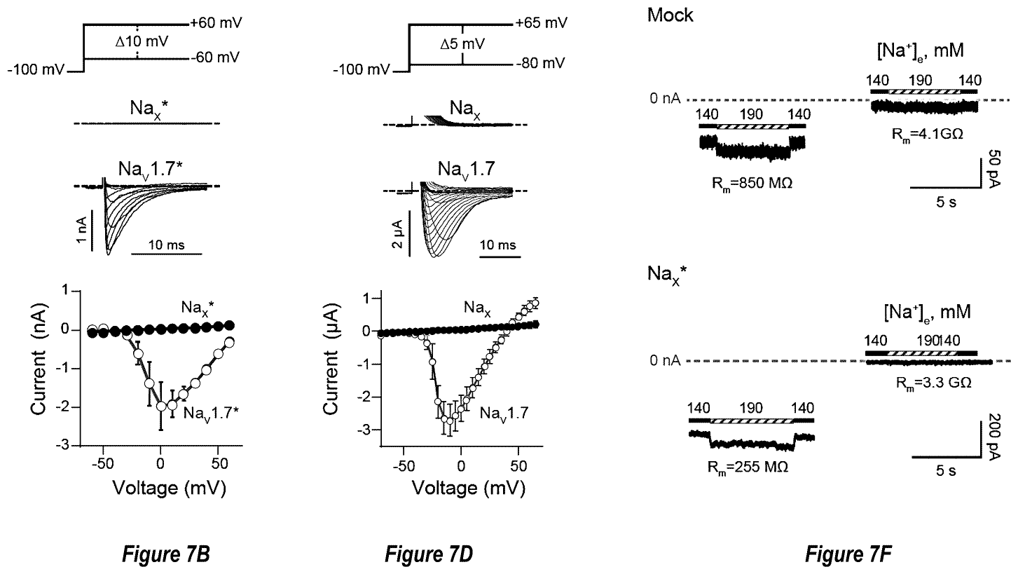

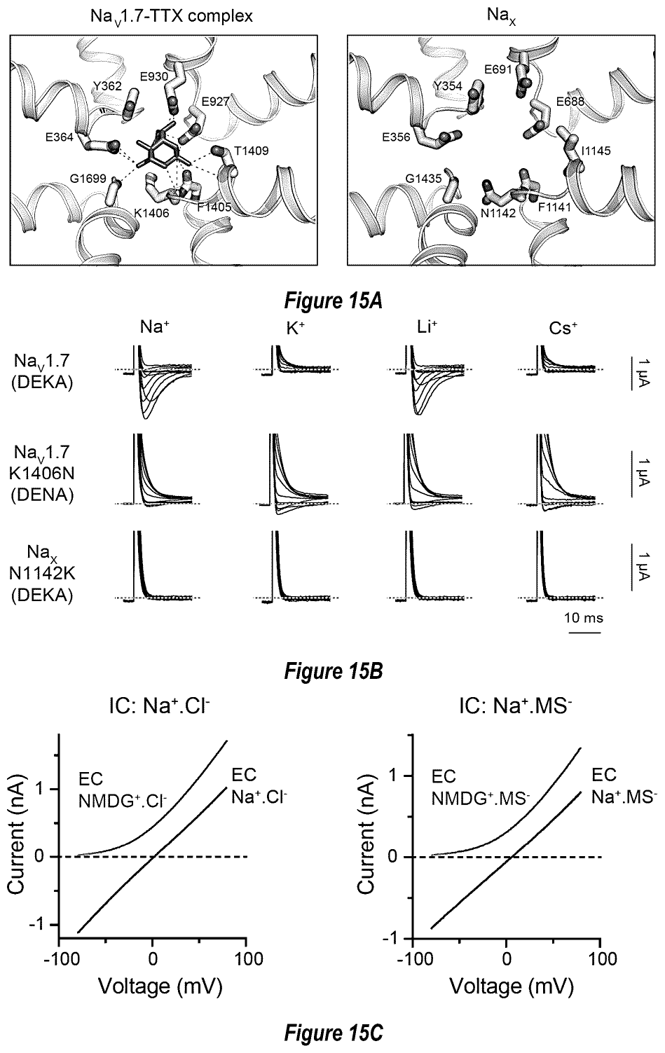

HEK293T cells expressing Na_{X}- or Na_{X}-QTT-eGFP-2×FLAG were seeded on glass cover slips an hour before whole-cell patch-clamping experiments. For a more physiological condition (FIGS. 4D and 7B), the extracellular solution contained 150 mM NaCl, 5 mM KCl, 0.5 mM CaCl₂, 1.2 mM MgCl₂, 10 mM HEPES, and 13 mM D-(+)-glucose (pH 7.4) with NaOH, ˜320 mOsm/L, and the intracellular solution contained 140 mM CsCl, 10 mM CsF, 5 mM EGTA, 10 mM HEPES, and 2 mM Na₂ATP (pH 7.2) with CsOH, ˜304 mOsm/L. To determine if Na_{X}-QTT was cation-permeable, we first substituted all extracellular cations with NMDG⁺ (FIG. 4E). Extracellular solution contained 150 mM NMDG and 10 mM HEPES (D-(+)-glucose was added accordingly to achieve osmolarity ˜325 mOsm/L and pH was adjusted to 7.4 with hydrochloric acid). To determine if Na_{X}-QTT was anion-permeable, we substituted all Cl⁻ ions in both intra- and extracellular solutions with the large anion methanesulfonate (MS⁻) (FIG. 15C). The extracellular solution contained 150 mM NaMS and 10 mM HEPES (D-(+)-glucose was added accordingly to achieve osmolarity ˜325 mOsm/L, and pH was adjusted to 7.4 with NaOH). The intracellular solution contained 136 mM NaMS, 10 mM NaF, 5 mM EGTA, 10 mM HEPES, and 2 mM Na₂ATP (pH 7.2 with NaOH, ˜309 mOsm/L). The extracellular NMDG solution contained 150 NMDG and 10 mM HEPES (D-(+)-glucose was added accordingly to achieve osmolarity ˜325 mOsm/L, and pH was adjusted to 7.4 with methanesulfonic acid).

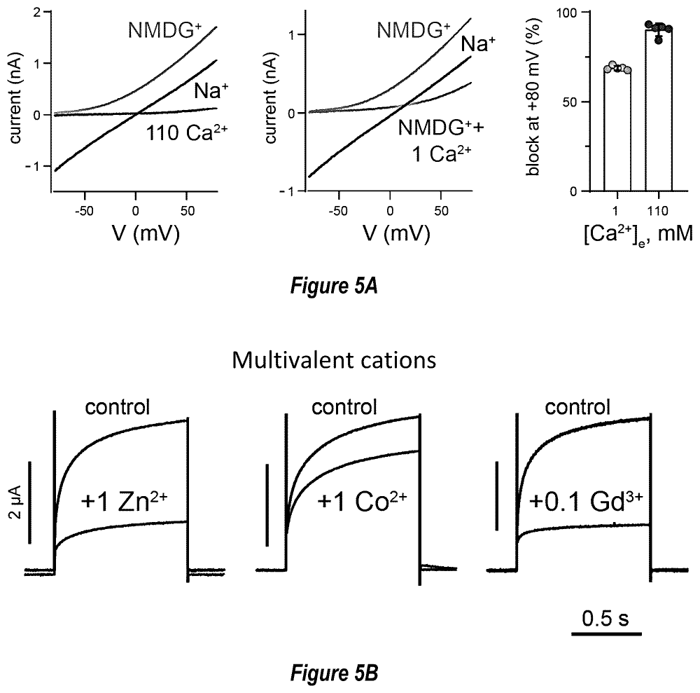

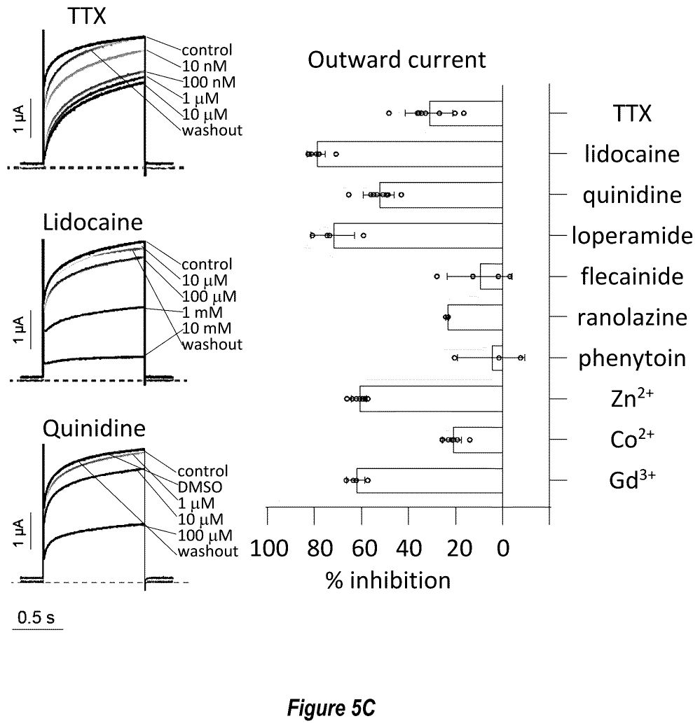

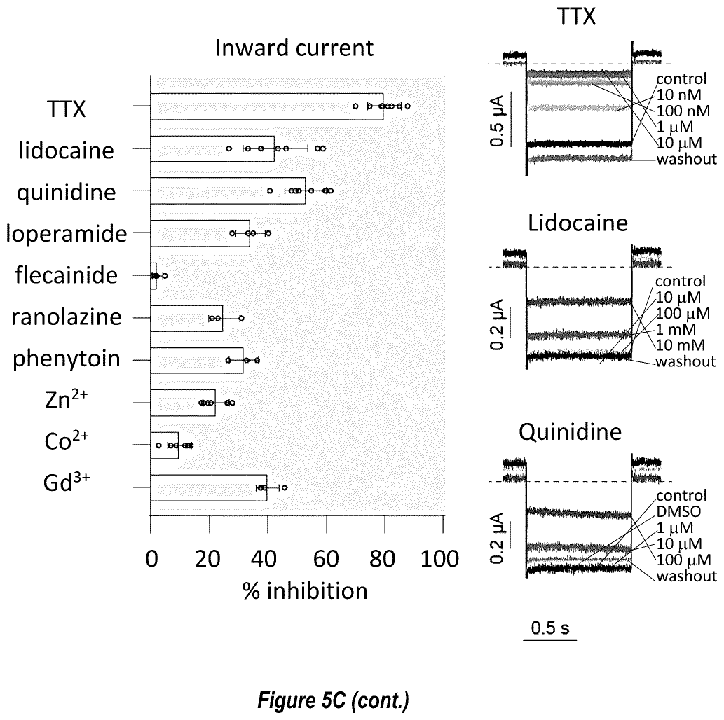

For ion-selectivity experiments (FIGS. 4E and 5A) (i) involving monovalent cations, the extracellular solution contained 150 mM XCl, 10 mM HEPES, and D-(+)-glucose was added accordingly to achieve osmolarity ˜325 mOsm/L, and the pH was adjusted to 7.4 with XOH, where X indicates the cation of interest (Na⁺, K⁺, Li+ or Cs+); (ii) involving Ca²⁺, the extracellular solution contained 110 mM CaCl₂, 10 mM HEPES, and D-(+)-glucose was added accordingly to achieve osmolarity ˜325 mOsm/L, and the pH was adjusted to 7.4 with Ca(OH)₂; (iii) the intracellular solution contained 136 mM NaCl, 10 mM NaF, 5 mM EGTA, 10 mM HEPES, and 2 mM Na₂ATP (pH 7.2) with NaOH, ˜309 mOsm/L.

### Example 1.12—Data Analysis

For data analysis, raw current traces were typically filtered using 8-pole Bessel low-pass filter at 500-800 Hz. Current traces were subjected to factor 5 data reduction for display in figures. Data are presented as mean±standard deviation (SD) from at least 3 cells from at least two batches of cells. For ion-selectivity experiments performed with Na⁺ intracellular solution (FIGS. 4E and 5A), liquid junction potential (LJP) was measured with reference to the Na⁺ intracellular solution and corrected after recording. Relative ion permeabilities (PNa/PX) were calculated using the equation P_{X}/P_{Na}=[Na⁺]_{IC} exp(EᵣₑᵥF/RT)/[X⁺]_{EC} (X=Na, K, Li or Cs), where F is Faraday's constant, R is the gas constant, T=274 K, and Eᵣₑᵥs were measured from the −80- to +80-m V ramp (corrected for LJP).

### Example 2—Evaluation of Human Na_{X} Function

Reliable ionic currents were not detected following expression of human Na_{X} in HEK293T cells or Xenopus laevis oocytes in response to changes in membrane voltage (FIGS. 1A-D, 7A-D, and 8A-D). The application of classical Na_{V} channel activators like aconitine, veratridine, pompilidotoxins, blood-depressing substance-I, and neurotoxin-II during different voltage-step protocols in oocytes did not invoke any clear human Na_{X}-mediated currents (FIGS. 1B, 1D, and 8A-B). Despite robust surface expression of human Na_{X}, co-expression with Na_{V} or voltage-gated calcium (Ca_{V}) channel auxiliary-subunits (i.e. β1, β3, α2δ1, γ2) also failed to produce dependable currents (FIGS. 1C, 1D, 7A, 7C and 8C-D). These results considerably extend prior findings that Na_{X} does not operate as a conventional voltage-gated channel^{14,15}.

Increases in the extracellular sodium concentration, [Na⁺]ₑ, have been reported to activate a non-inactivating Na⁺ current in glial and neuroblastoma (Neuro-2a) cell lines expressing murine Na_{X}. Murine Na_{X} has also been suggested to interact with the Na⁺/K⁺-ATPase and synapse-associated protein 97 (SAP97) at the plasma membrane. Despite surface expression and our ability to also co-immunoprecipitate the ATP1A1 a-subunit under some conditions (Table 2), no reliable inward Na⁺ currents were detected in oocytes co-expressing human Na_{X} with the Na⁺/K⁺-ATPase subunits and SAP97 upon exposure to 200 mM [Na⁺]ₑ (FIGS. 1C and 1D).

TABLE 2
Naₓ Channel Sample Analyzed

| Protein ID: | #PSMs | % Coverage |
|---|---|---|
| SCN7A + SCN1B | | |
| SCN7A | 254 | 36 |
| SCN1B | 62 | 40 |
| HSP71_HUMAN | 32 | 43 |
| DNJC7_HUMAN | 27 | 46 |
| F10A1_HUMAN | 22 | 31 |
| SCN7A + SCN3B | | |
| SCN7A | 234 | 35 |
| SCN3B | 19 | 44 |
| HSP71_HUMAN | 24 | 30 |
| DNJC7_HUMAN | 19 | 31 |
| AT1A1_HUMAN | 24 | 21 |

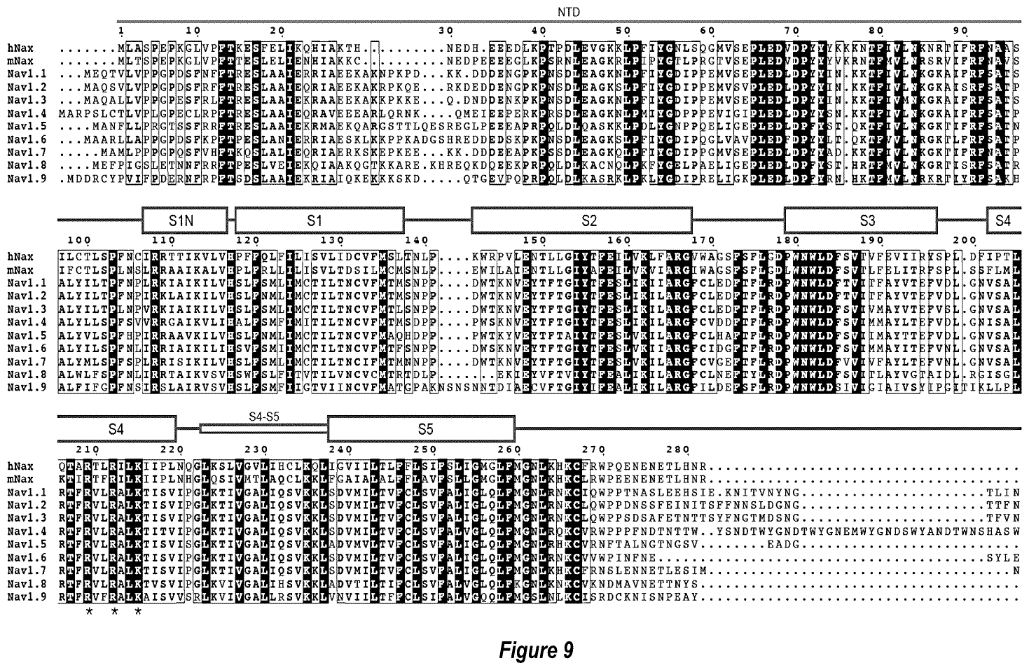

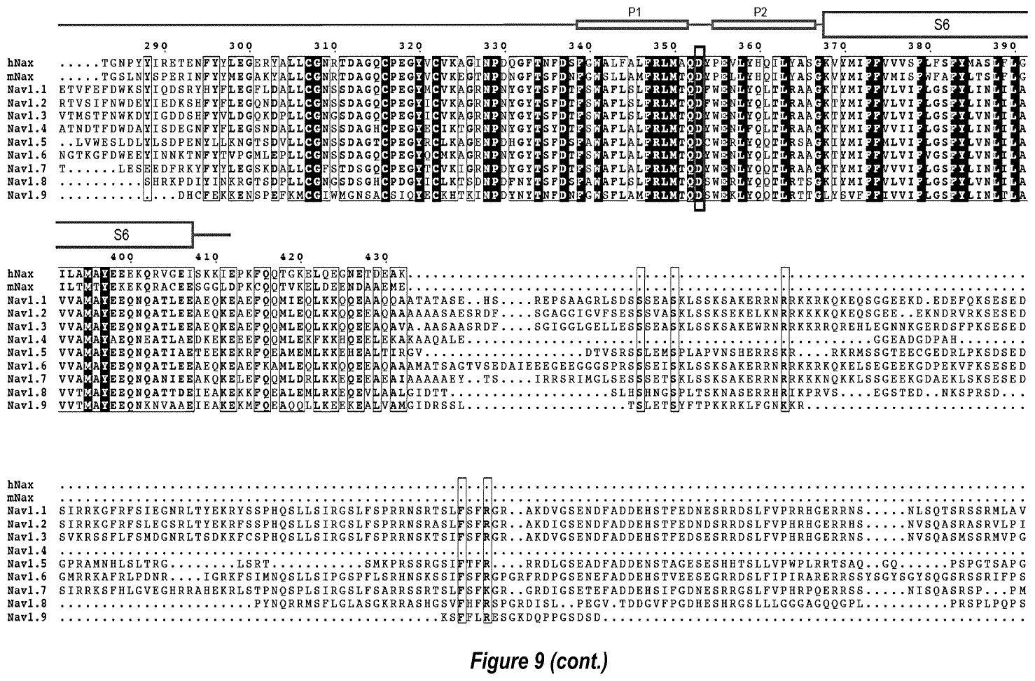

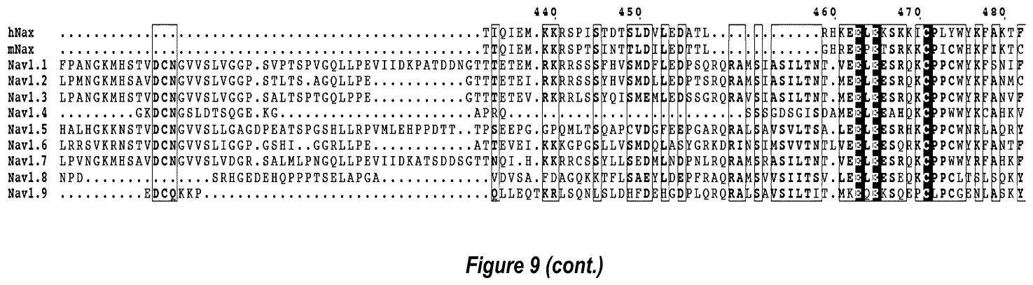

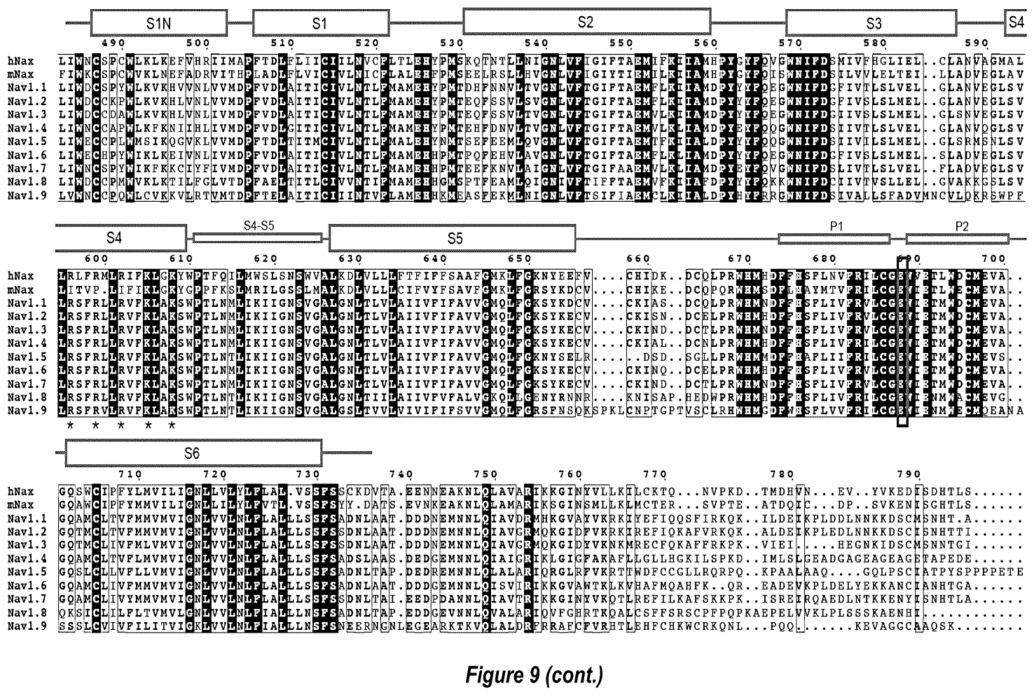

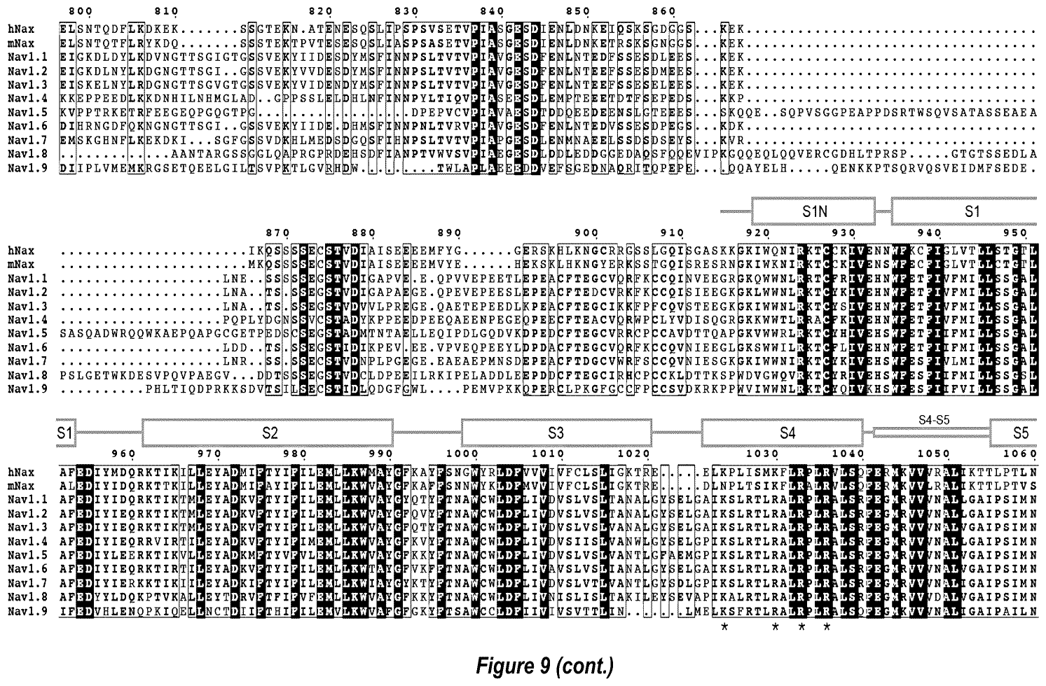

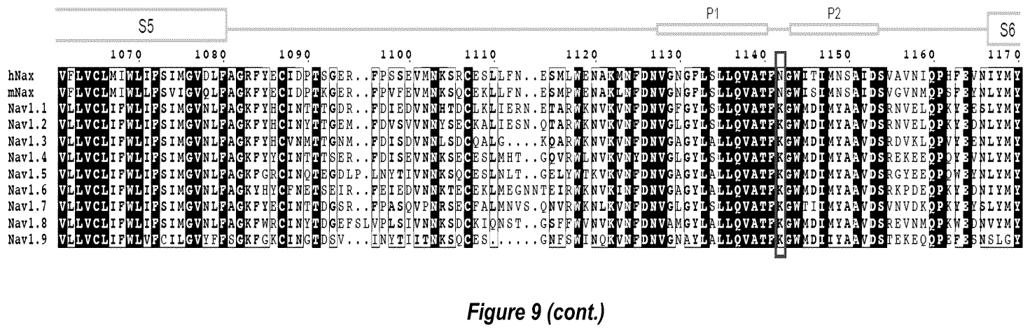

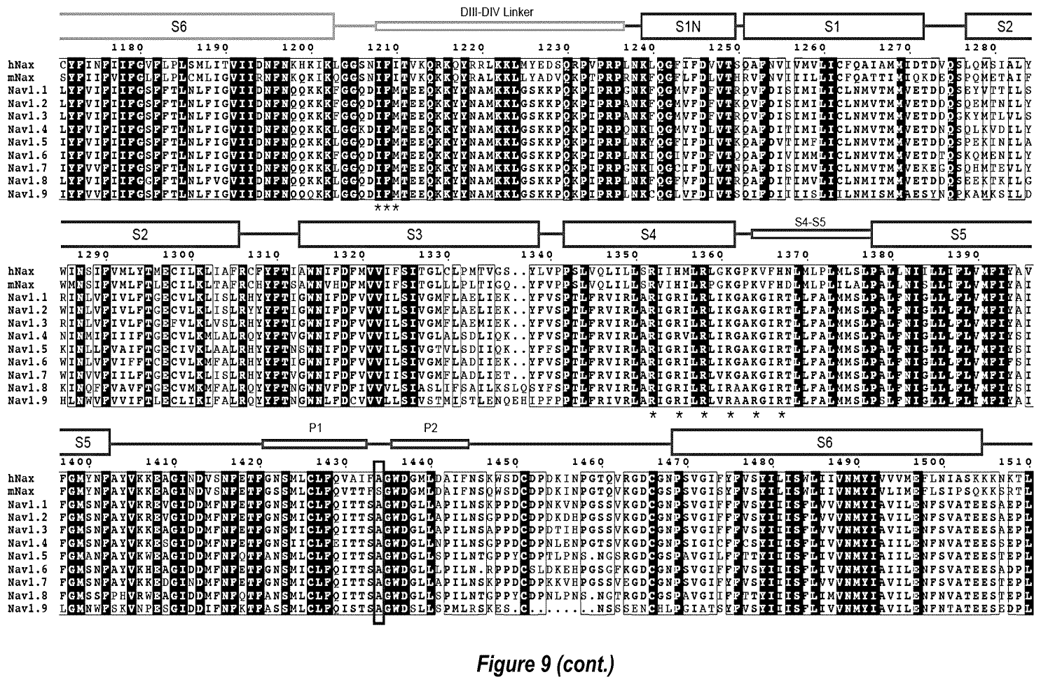

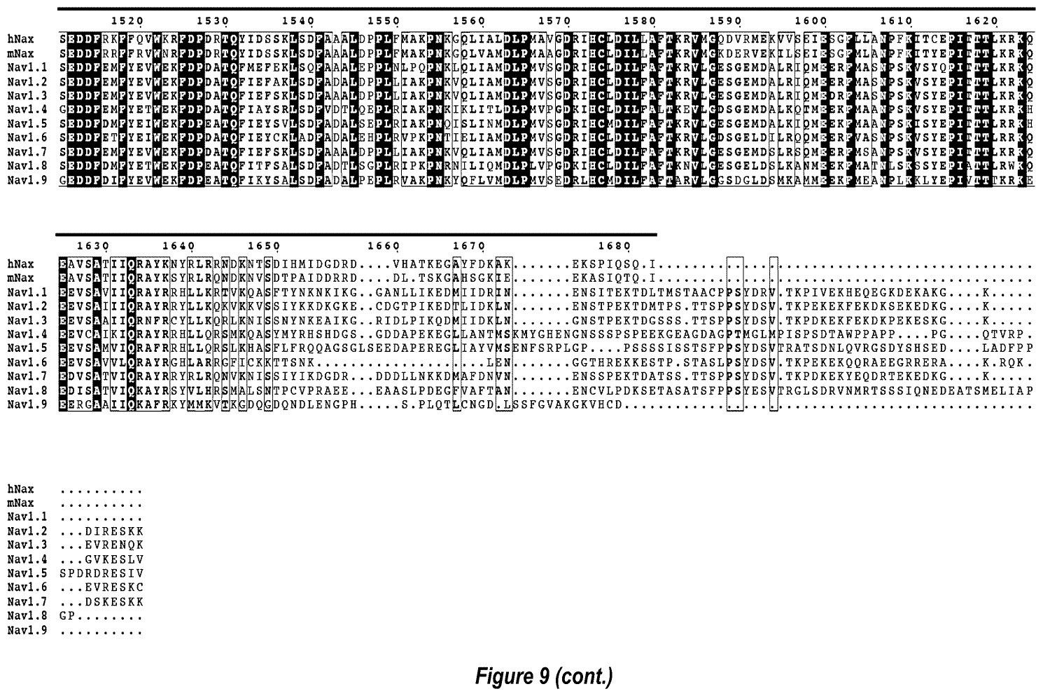

When human Na_{X} was expressed in Neuro-2a cells, we observed small and flickery inward currents upon changes in [Na⁺]ₑ from 140 to 190 mM in the whole-cell patch-clamp configuration (FIGS. 1E-G and 7E-F). Neuro-2a cells expressing the Na_{V}1.7 channel showed similar flicker behavior following osmolarity changes, indicating that this phenomenon was not specific to cells expressing human Na_{X} (FIGS. 1E-G). A non-inactivating inward current was only observed when seal resistances were low (<1 G (2), and this phenotype was found in both mock- and human Na_{X}-transfected Neuro-2a cells (FIG. 7F). Overall, it is unclear if our findings reflect a functional divergence between human and murine Na_{X} channels (FIG. 9), or slight differences in experimental protocols, but we failed to measure any reliable currents through human Na_{X}. We next considered if structure determination of human Na_{X} might provide insight into its function or regulation.

### Example 3—Structure Determination of the β3-Na_{X} Complex in Lipid Nanodiscs

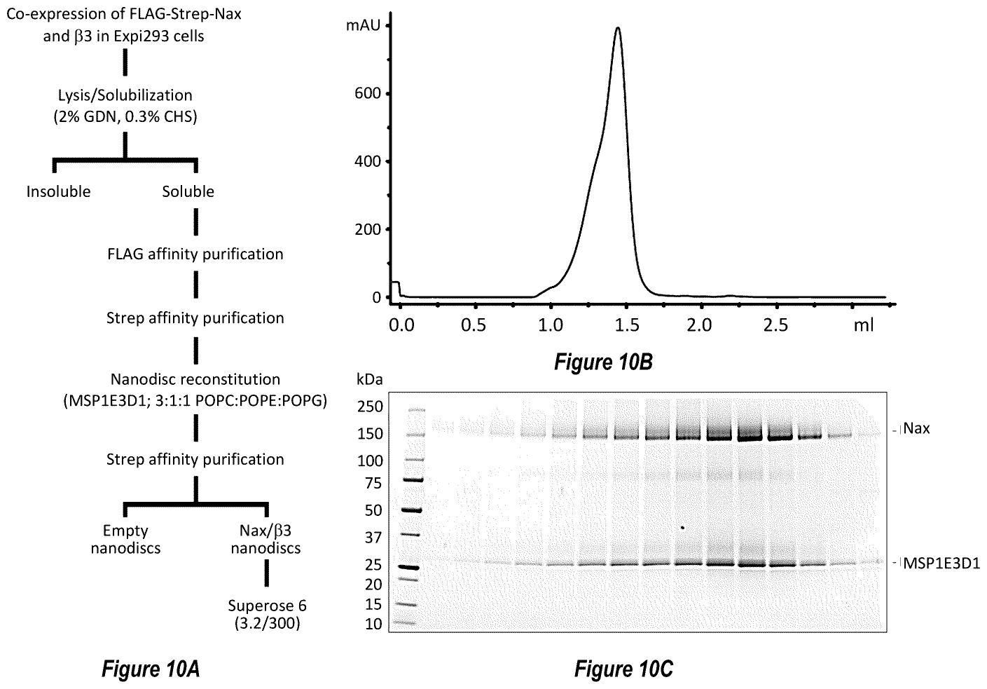

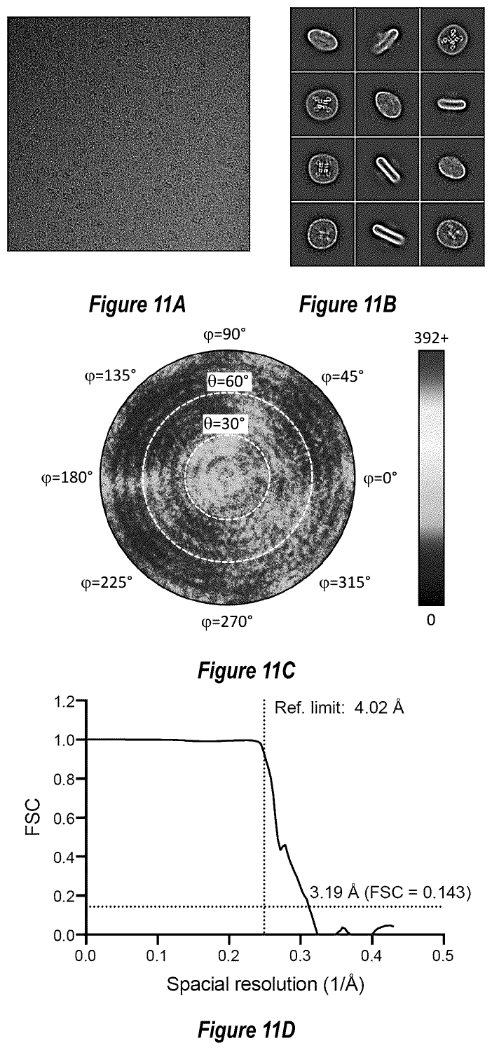

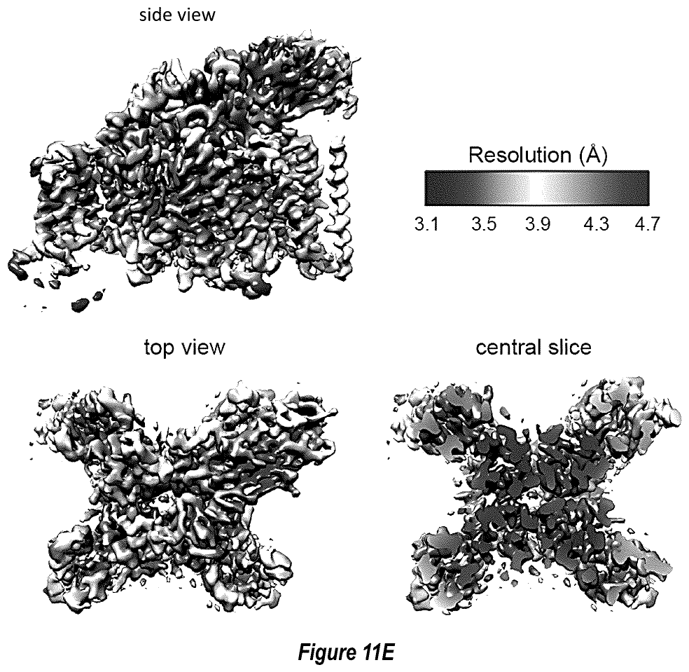

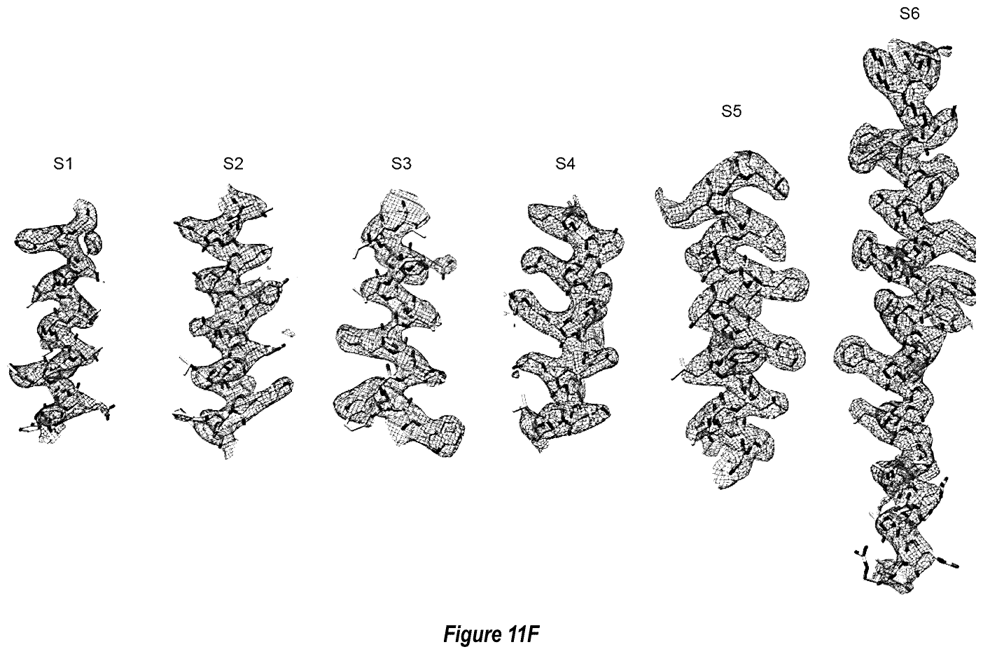

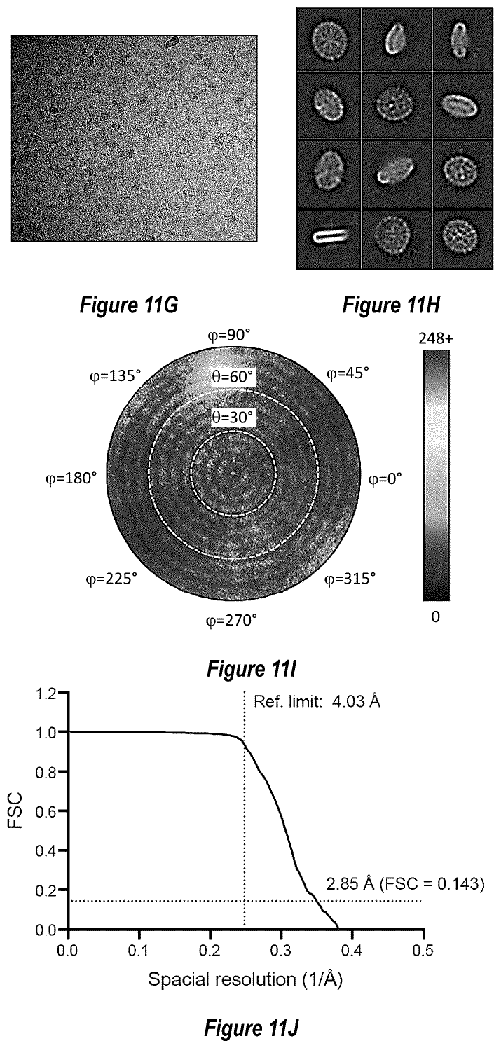

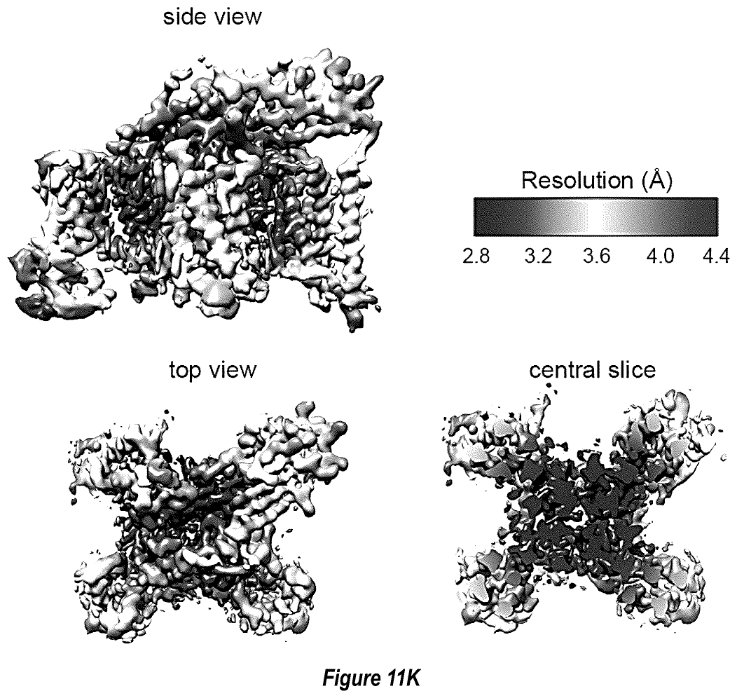

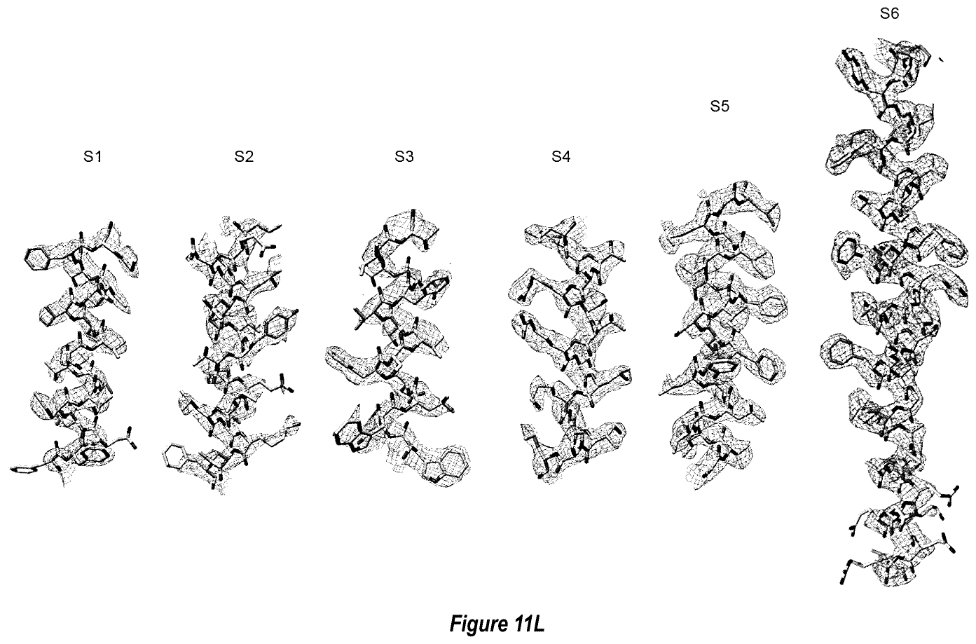

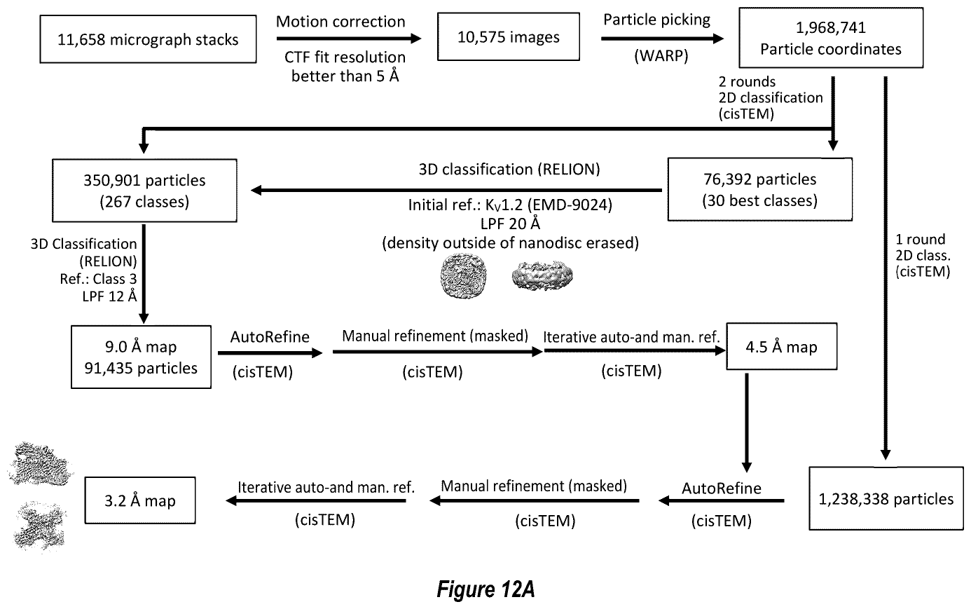

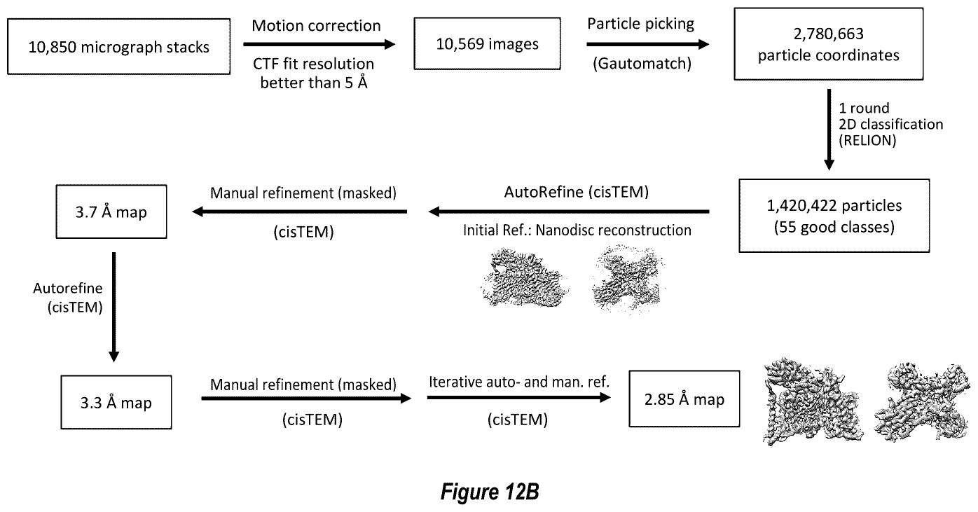

Human Na_{X} was recombinantly expressed in HEK293 cells and purified in the mild detergent glyco-diosgenin (FIGS. 10A-C). Inspired by reports that the Na_{V}1.7 channel forms stable complexes with auxiliary β-subunits²³⁻²⁵, β1- or β3-subunits were co-expressed and found to co-purify with Na_{X} (Table 2). To provide a more native environment, the β3-Na_{X} complex was reconstituted into nanodiscs using a common phospholipid mixture (FIGS. 10A-C). Prior to sample vitrification, the Na⁺ concentration was raised above the threshold reported to activate Na_{X}, to 200 mM. The resulting cryo-EM reconstruction of the human β3-Na_{X} complex extended to 3.2 Å resolution (FIGS. 11A-F, 12A, and Table 3) and represents the first structure of a eukaryotic Na_{V} channel family member determined in a reconstituted lipid membrane environment. In the first, title row of Table 3 below, the EMDB reference for β3-Na_{X}-nanodisc is EMDB-25919, and the PDB reference is PDB 7TJ8. The EMDB reference for β3-Na_{X}-GDN is EDMB-25920 and the PDB reference is PDB 7TJ9.

TABLE 3
Cryo-EM Data Collection, Refinement and Validation Statistics

| | β3-Naₓ-nanodisc | β3-Naₓ-GDN |
|---|---|---|
| | (EMDB-25919) | (EMDB-25920) |
| | (PDB 7TJ8) | (PDB 7TJ9) |
| Data collection and processing | | |
| Magnification | 165,000 | 105,000 |
| Voltage (kV) | 300 | 300 |
| Electron exposure (e-/Å²) | 48.579 | 64.009 |
| Defocus range (μm) | 0.5-1.5 | 0.5-1.5 |
| Pixel size (Å) | 0.849 | 1.1648 |
| Symmetry imposed | C1 | C1 |
| Initial particle images (no.) | 1,968,741 | 2,780,663 |
| Final particle images (no.) | 1,238,338 | 1,420,422 |
| Map resolution (Å) | 3.2 | 2.9 |
| FSC threshold | 0.143 | 0.143 |
| Map resolution range (Å) | 3.2-46.6 | 2.9-33.13 |
| Refinement | | |
| Initial model used | 6AGF | β3-Naₓ-nanodisc |
| (PDB code) | | |
| Model resolution (Å) | 3.4 | 3.1 |
| FSC threshold | 0.5 | 0.5 |
| Model resolution range (Å) | 3.2-46.6 | 2.9-33.13 |
| Map sharpening B factor (Å²) | −90 | −90 |
| Model composition | | |
| Non-hydrogen atoms | 21502 | 22342 |
| Protein residues | 1242 | 1273 |
| Ligands | 18 | 23 |
| B factors (Å²) | | |
| Protein | 67.58 | 77.83 |
| Ligand | 64.16 | 70.41 |
| R.m.s. deviations | | |
| Bond lengths (Å) | 0.004 | 0.006 |
| Bond angles (°) | 0.906 | 0.611 |
| Validation | | |
| MolProbity score | 1.78 | 1.66 |
| Clashscore | 5.79 | 5.15 |
| Poor rotamers (%) | 0.80 | 0.09 |
| Ramachandran plot | | |
| Favored (%) | 93.72 | 94.85 |
| Allowed (%) | 5.79 | 5.15 |
| Disallowed (%) | 0.49 | 0.00 |

### Example 4—Overall Structure of the β3-Na_{X} Channel Complex

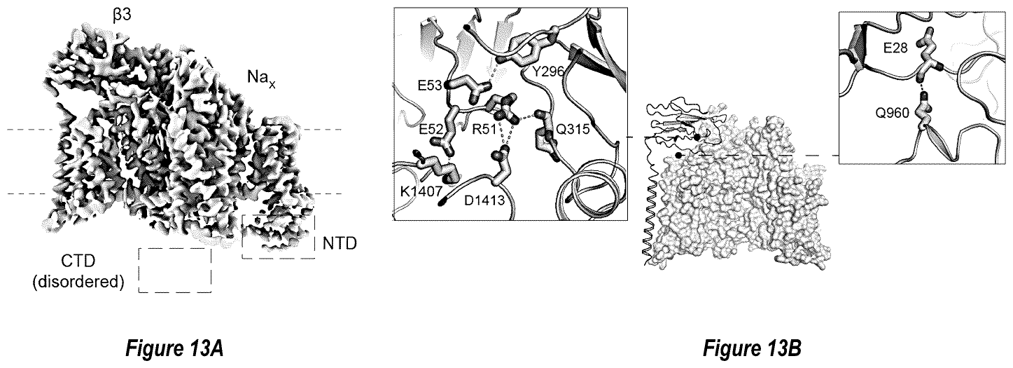

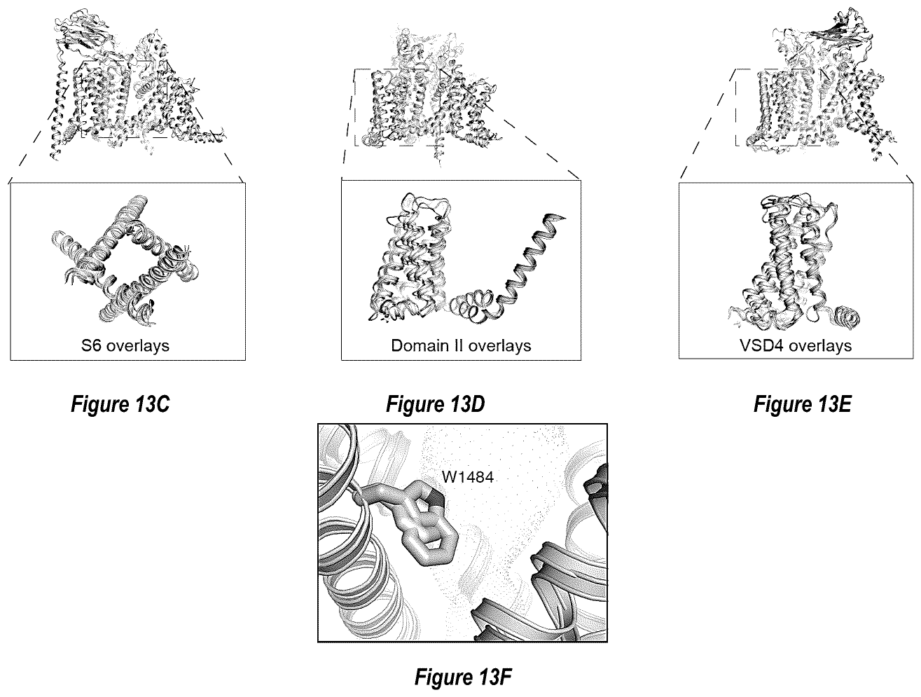

The β3-Na_{X} channel complex resembles a four-leaf clover with the VSLDs arranged in a domain-swapped organization around the central pore module (FIG. 1H), which conforms to the architecture shared among Na_{V}, Ca_{V} and NALCN channels. In Na_{X}, a soluble intracellular amino-terminal domain is docked beneath VSLD1, the intracellular DIII-DIV linker is bound alongside the pore, and the intracellular carboxyl-terminal domain (CTD) is not well resolved (FIG. 13A), features reminiscent of available human Na_{V} channel structures. On the extracellular side, two and four N-linked glycans are found to extend from the Na_{X} and β3 subunits, respectively.

The β3-subunit forms extensive interactions by wedging its immunoglobulin-domain between elaborated extracellular loops of the Na_{X} pore module and positioning its single transmembrane helix against VSLD3 (FIGS. 1H and 13B). Although contemporary β1- or β3-auxiliary subunits appear to form stable complexes with Na_{X} (Table 2), its pore module does not possess the cysteine residue required to form covalent complexes with β2- or β4-subunits^{23,27}.

The overall architecture of human Na_{X} reconstituted in lipid nanodiscs is highly similar to Na_{V}1.1, Na_{V}1.2, Na_{V}1.4, Na_{V}1.5 and Na_{V}1.7 channel structures determined in detergent^{23,28-31} (FIG. 13C). Conversely, Na_{X} does display substantial deviations in regions that are frequently implicated in Na_{V} channel gating, including a ˜5 Å positional shift of VSLD4, and >6 Å displacements along the DII S4-S5 linker (FIGS. 13D and 13E). It seems plausible that the observed structural changes in Na_{X} are a consequence of its high sequence divergence and contribute to its distinct function (FIG. 9).

### Example 5—the Na_{X} Pore Module is in a Non-Conductive State

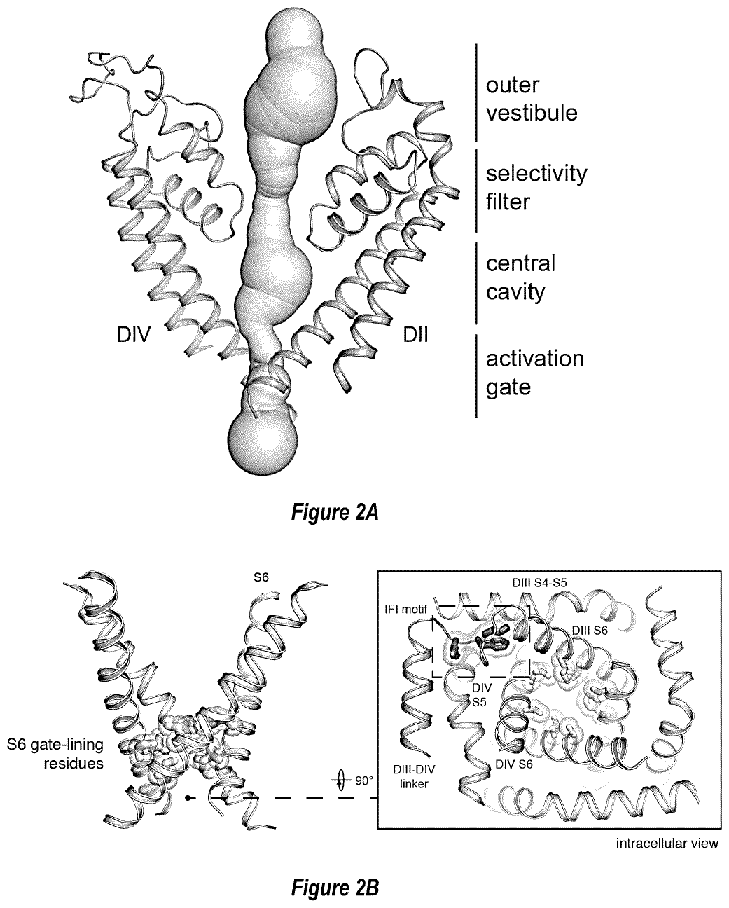

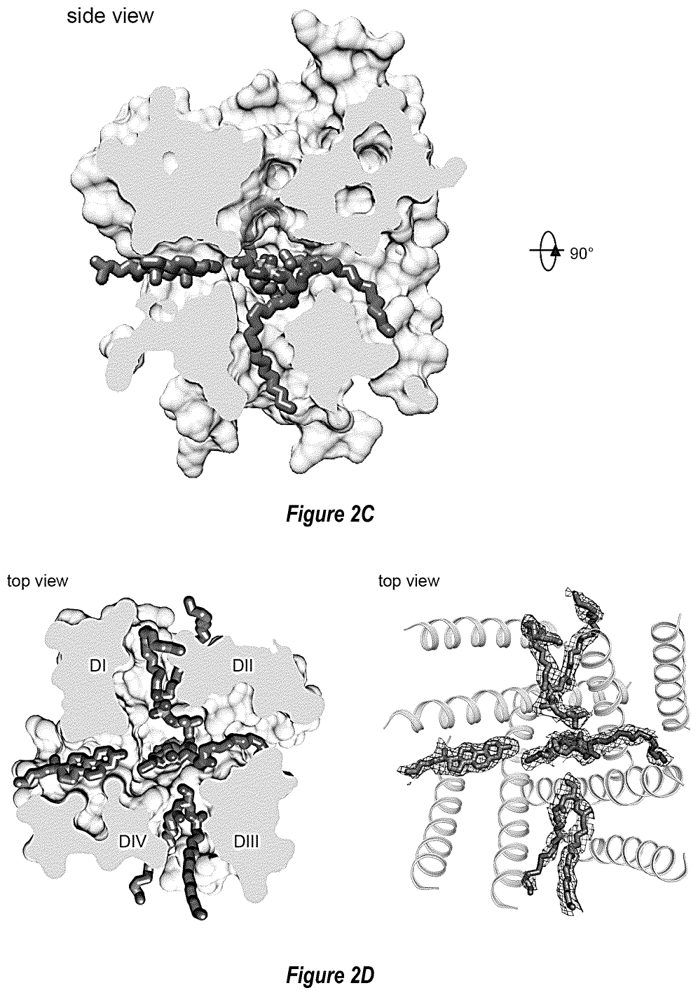

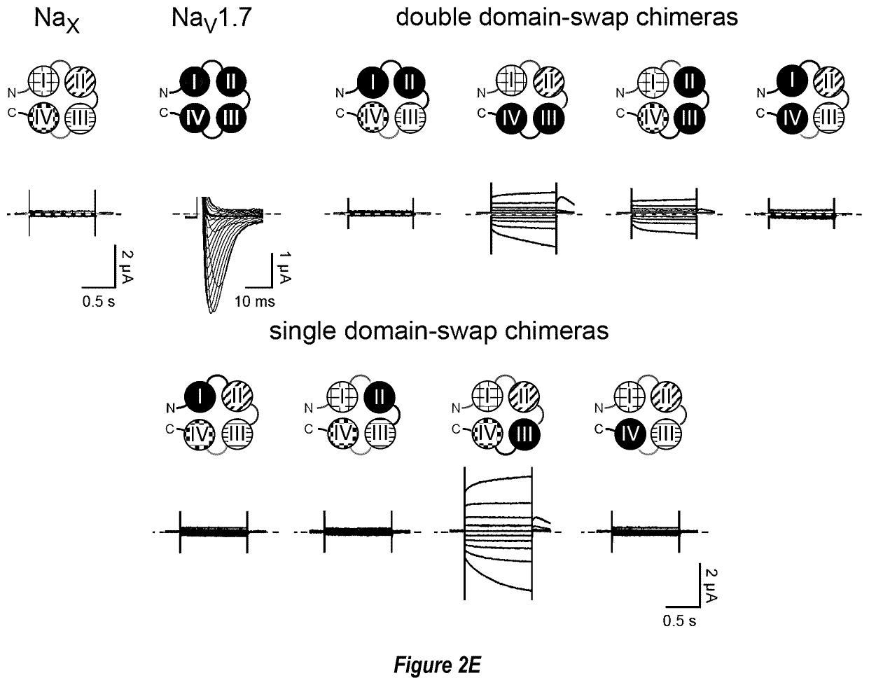

The Na_{X} pore module contains an outer vestibule, an ion selectivity filter, a central cavity, and an S6-activation gate (FIG. 2A). The S6-gate is narrow and lined by a double-ring of hydrophobic side-chains that would form a barrier to the passage of hydrated ions, which unequivocally defines a non-conductive state (FIGS. 2A, 2B and 13C)^{23,28-31}. However, because our Na_{X} structure was determined under high Na⁺ concentrations, these findings appear inconsistent with reports that murine Na_{X} forms a Na⁺-activated, non-inactivating channel. Instead, the non-conductive state observed for the pore module aligns perfectly with predictions from our physiological studies on human Na_{X} (FIGS. 1A-F, 7A-F and 8A-D).

The Na_{X} central cavity is similar in volume to those in Na_{V} channels, although the DIV-S6 phenylalanine commonly targeted by pore blocking drugs^{32,33} is replaced by DIV-Trp1484 (FIG. 13F). Na_{X} reveals four lateral fenestrations that penetrate the pore module, identifying membrane access pathways for hydrophobic drugs or lipids to directly enter into the central cavity (FIGS. 2C and 2D), as first suggested in Na_{V} channels^{28-30,34}. It is therefore conceivable that classic Na_{V} or Ca_{V} channel antagonists might target Na_{X} within the central cavity.

### Example 6—The Na_{X} DIII-DIV Linker Restricts S6-gate Dilation

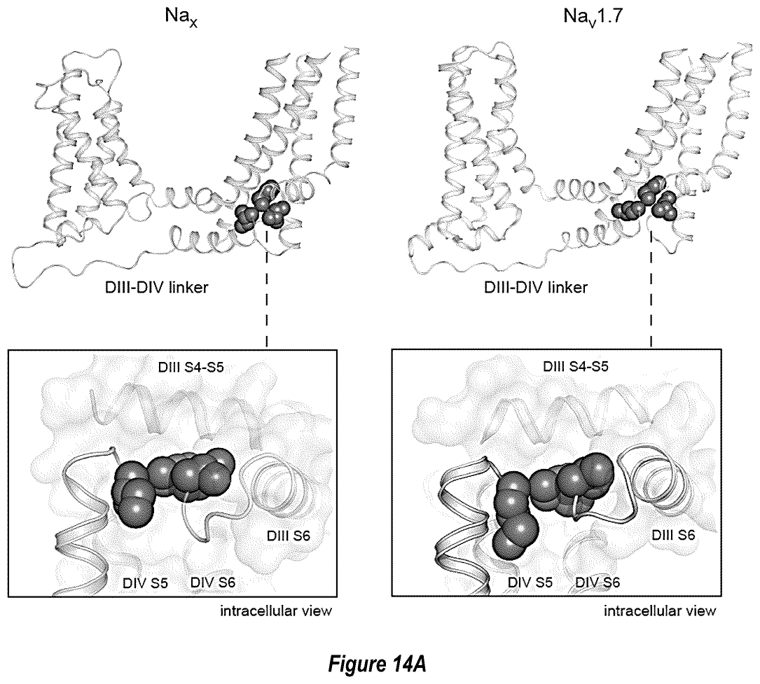

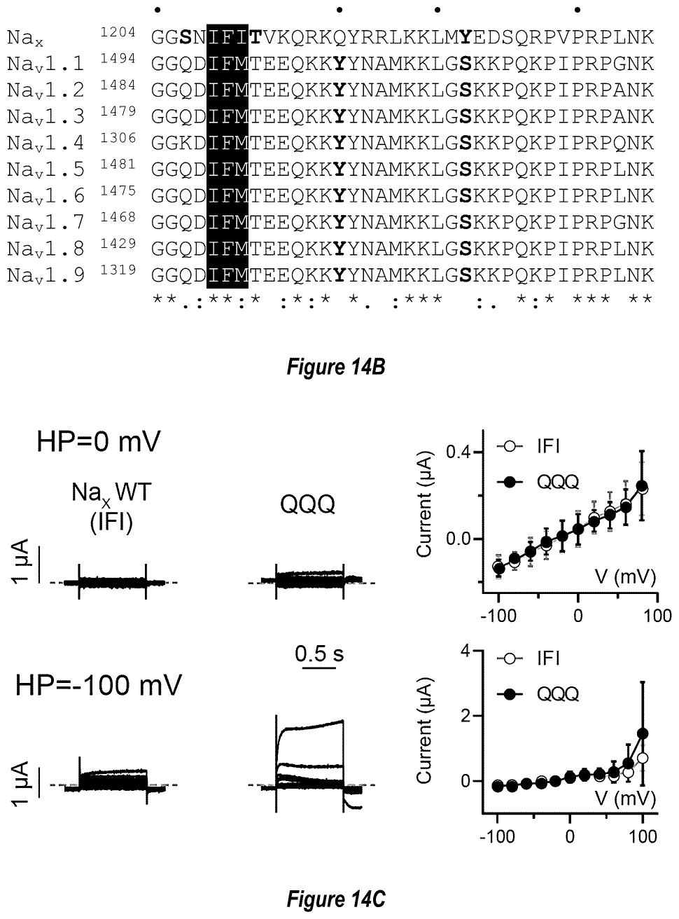

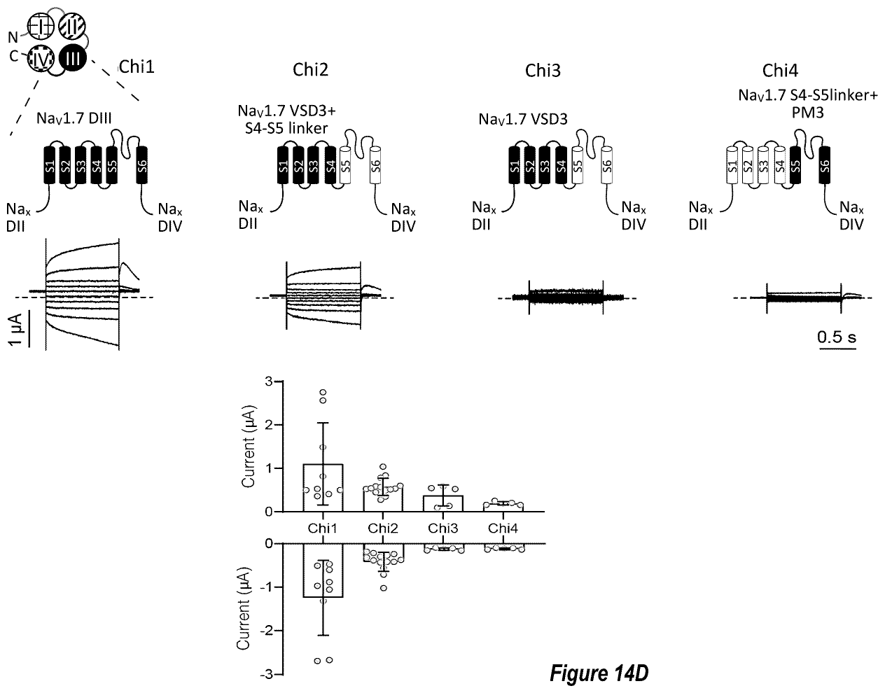

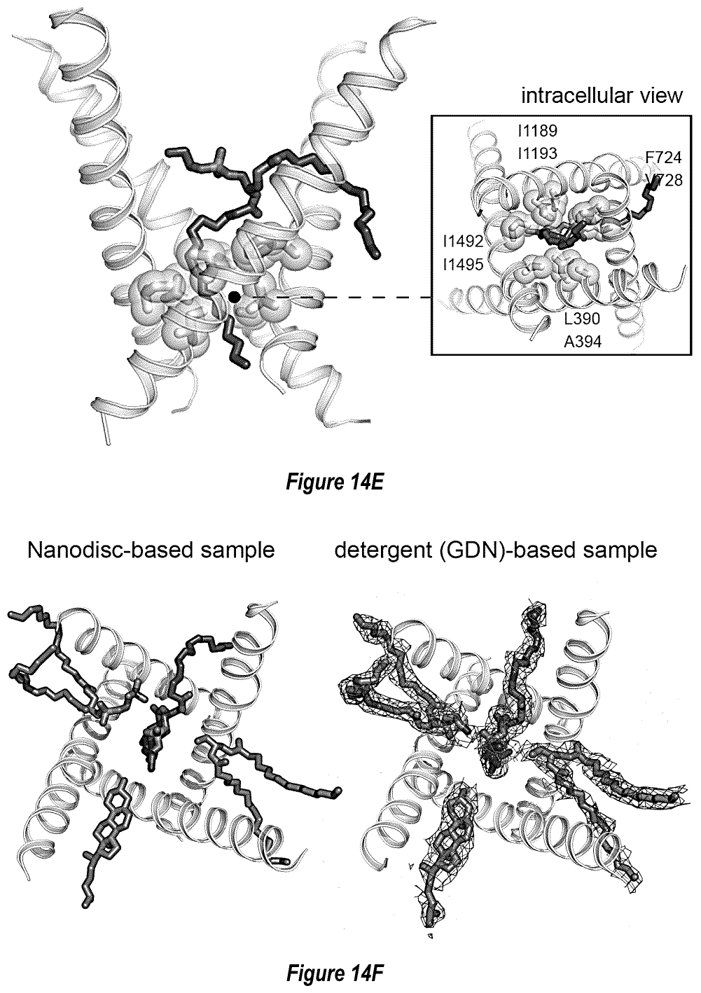

The intracellular DIII-DIV linker of Na_{X} interacts with a pore module receptor site in a manner which closely resembles the fast-inactivated state structure of human Na_{V} channels (FIGS. 2B and 14A). The Na_{X} IFI-motif is bound in-between the DIII-S4-S5 linker and the DIV-S5 and S6, analogous to complexes that restrict S6-gate dilation in Na_{V} channels (FIGS. 2B and 14A). Although the DIII-DIV linker of Na_{X} has long been recognized as a site of substantial sequence divergence (FIG. 14B), it clearly shares high structural homology with this key functional region in Na_{V} channels (FIG. 14A). Nevertheless, mutating the IFI-motif of Na_{X} into a classical non-inactivating Na_{V} channel QQQ-motif was insufficient to generate functional channels in injected oocytes (FIG. 14C), which further highlights the existence of distinct requirements to gate the Na_{X} channel.

We exploited Na_{X} and Na_{V}1.7 structural models to construct a panel of precisely-targeted chimeric channels to evaluate the determinants responsible for the non-conductive state of human Na_{X}. Double-domain DII-DIII and DIII-DIV Na_{V}1.7-Na_{X} chimeras and the single-domain DIII Na_{V}1.7-Na_{X} chimera produced large inward and outward currents when expressed alone in Xenopus oocytes (FIG. 2E). Ultimately, swapping both Na_{V}1.7-VSD3 and the associated DIII-S4-S5 linker were the minimal regions sufficient to transfer the leak channel phenotype (FIG. 14D). Because these regions are required to form the receptor site for the IFM-motif which restricts S6-dilation in Na_{V} channels (FIG. 14A), and their substitution into Na_{X} produces robust leak currents (FIGS. 2E and 14D), we propose that the DIII-DIV linker of Na_{X} serves to restrict S6-dialation and stabilize the non-conductive state of the pore module.

### Example 7—The Na_{X} Pore is Infiltrated by Membrane Lipids

By definition, a closed or inactivated state of an ion channel is non-conductive. In the β3-Na_{X} structure, four lipids penetrate through the lateral pore fenestrations to enter the central cavity above the narrow, hydrophobic S6-gate. These striking densities can be unambiguously assigned as three phospholipids and one cholesterol (FIGS. 2C and 2D). One phospholipid even straddles and seals the intracellular S6-activation gate (FIGS. 2C and 14E).

To evaluate if the lipid-nanodisc environment was responsible for the robust visualization of membrane lipids in β3-Na_{X}, we prepared a new sample in the detergent glyco-diosgenin without exogenous phospholipid supplementation and determined its structure to 2.85 Å resolution (FIGS. 11G-L, 12B and Table 3). We observed an identical Na_{X} pore structure penetrated by four well-defined co-purifying lipids (FIG. 14F). The ion conduction pathway in β3-Na_{X} is therefore unequivocally non-conductive in this pore module conformation because it is infiltrated and occluded by lipids (FIGS. 2A-D and 14E-F). The presence of well-resolved, co-purifying lipids bound within Na_{X} may reflect the intrinsic stability of its non-conductive state and hydrophobicity of the S6 gate and central cavity.

### Example 8—Targeted Pore-Wetting Mutations can Activate Human Na_{X}

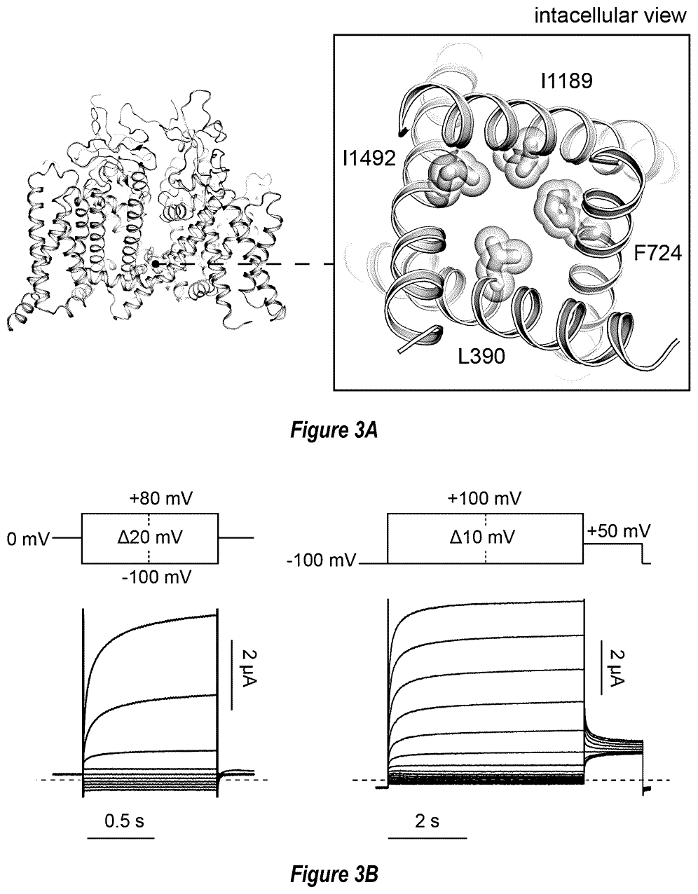

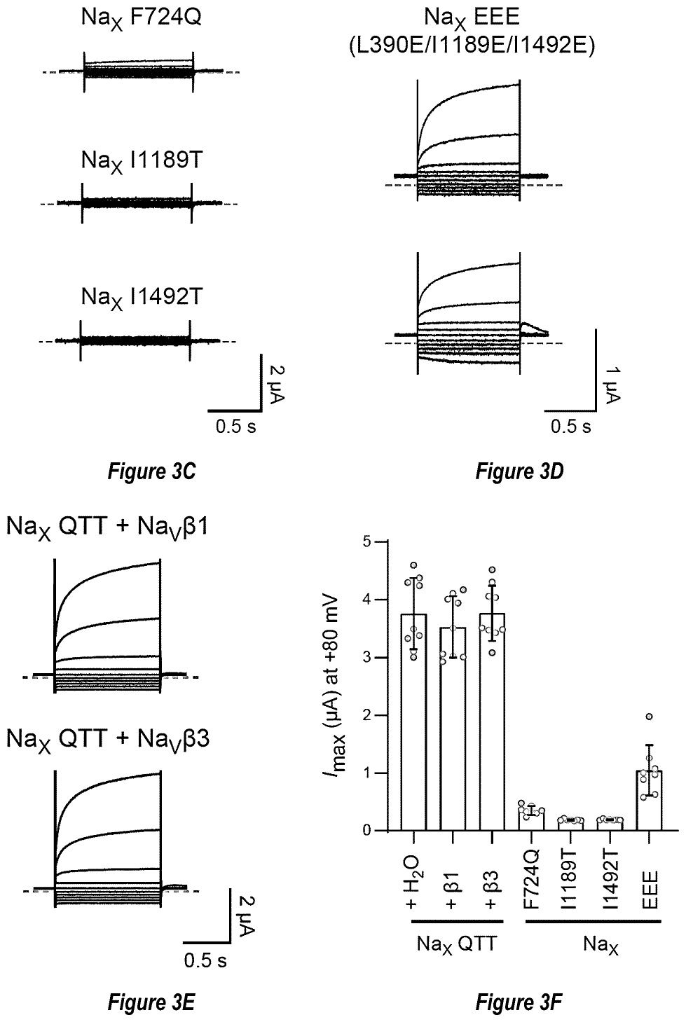

Based on our collective observations that the S6-gate is narrow, hydrophobic, and lipid-bound, and that disruption of the IFI-motif receptor site may promote S6-dilation, we reasoned that the targeted introduction of polar residues around the S6-gate might promote pore hydration, destabilization of bound lipids, and transition of Na_{X} to an open, conductive state (FIGS. 3A-F). Accordingly, a triple-mutant Na_{X} channel construct (F724Q-I1189T-I1492T) containing polar substitutions at three S6-gate lining positions, Na_{X}-QTT, produced robust ionic currents when expressed alone in Xenopus oocytes in response to voltage-step protocols (FIGS. 3A and 3B). The corresponding single-point mutant Na_{X} channel constructs failed to produce robust currents, signifying a pore-wetting threshold exists to achieve S6-gate dilation through destabilization of the non-conductive pore module state (FIG. 3C). The L390E-I189E-I1492E triple-mutant Na_{X} channel construct (Na_{X}-EEE) also displayed function but with much lower current amplitudes and higher current variability relative to Na_{X}-QTT (FIGS. 3D and 3F).

In injected oocytes, Na_{X}-QTT currents are outward-rectifying with no signs of inactivation even during long (5 sec) depolarization test pulses (FIG. 3B). Because Na_{X} can interact stably with the β1- or β3-subunits (FIG. 1H and Table 2), the functional effects of these auxiliary subunits were evaluated. No difference in current amplitudes or kinetics were observed when Na_{X}-QTT was expressed in the presence of the β1- or β3-subunits, respectively (FIGS. 3E and 3F). In HEK293T cells, Na_{X}-QTT also behaves as a non-inactivating channel in whole cell patch-clamp experiments when expressed with a C-terminal GFP-2×FLAG tag (see below). Critically, the Na_{X}-QTT channel construct contains only three mutations targeted around the intracellular S6-gate (FIG. 3A), far removed from the selectivity filter and other important channel regions, so below we characterize Na_{X}-QTT as a proxy to evaluate ion selectivity and pharmacology of the human Na_{X} channel.

### Example 9—The Na_{X} Selectivity Filter

The unique ion selectivity filter of Na_{X} (DENA-motif) is supported by a local architecture that differs radically from the scaffold which supports the classic DEKA-motif in Na_{V} channels (FIGS. 4A-C). Four landmark tryptophan residues in Na_{V} channels form interdomain interactions involving three conserved threonine side-chains (FIG. 4B)^{23,28-30,35,36}. In Na_{X}, Pro355 replaces the DI tryptophan, while Ala351 and Ile1432 substitute for the DI and DIV threonine counterparts, respectively (FIG. 4B). Substitutions at positions equivalent to Na_{X} Pro355 and Ile1432 are in Na_{V}1.5 are pathogenic, highlighting the potential functional relevance of these alterations. Consequently, the Na_{X} selectivity filter is wider and more electronegative than the selectivity filter in Na_{V} channels (FIG. 4B).

At the DENA-motif of Na_{X}, Asp353 (DI) hydrogen bonds to the backbone of DII-Trp689, Glu688 (DII) points into the ion permeation pathway, and Asn1142 (DIII) bonds to the DIV-Phe1433 carbonyl to impose a ˜1.5 Å radial shift onto Ala1434 (DIV) compared to the DIV alanine in Na_{V} channels (FIGS. 4A and 4C). The relative displacement of Ala1434 is accommodated by DI-Pro355 in Na_{X}, which also permits DI-Tyr359 to reorient ˜90° to fill the volume vacated by the absent conventional DI tryptophan (FIG. 4C). This structural remodeling displaces DI-Glu356 and DII-Glu691 in the outer vestibule by 2.5-3.5 Å, which modifies the canonical tetrodotoxin (TTX)-binding site and may highlight the potential to discover Na_{X}-selective inhibitors (FIG. 15A).

### Example 10—Na_{X} does not Discriminate Monovalent Cations

The signature DIII-Asn1142 side-chain dramatically alters the structure and chemical profile of the Na_{X} selectivity filter relative to the conventional DIII-lysine that plays a key role in establishing Na⁺-selectivity in Na_{V} channels (FIGS. 4A-C). Accordingly, we introduced a DENA-motif into the human Na_{V}1.7 channel through a single mutation and observed a pronounced loss in Na⁺ and Lit selectivity in injected Xenopus oocytes, whereas measurable K+ currents with unusual kinetics were recorded (FIG. 15B). In reciprocal experiments, mutational transfer of a canonical DEKA-motif into Na_{X} produced no measurable currents (FIG. 15B), confirming that molecular features beyond the Na_{X} DENA-motif contribute to its activation.

We examined currents from the Na_{X}-QTT construct expressed in HEK293T cells and found that when all cations within the extracellular solution were replaced with the large cation N-methyl-D-glucamine (NMDG), the reversal potential shifted from +5 mV to below −100 mV (FIG. 4D), establishing that this construct behaves as a cation channel, and that large cations do not permeate. Replacement of all extracellular Cl-ions with the large anion methansulfonate did not change the current profile (FIG. 15C), indicating that Na_{X} behaves as a cation-selective channel, which is consistent with the size and electronegative profile of the selectivity filter (FIG. 4B). As expected, using a ramp protocol (−80 to +80 mV) with a simple Na⁺ intracellular solution (150 mM), the reversal potential was close to 0 mV under symmetrical NaCl/NaCl conditions (FIG. 4E). Notably, only slight changes in the reversal potentials were measured when cells were exposed to different extracellular monovalent cations (150 mM), establishing that Na_{X}-QTT does not effectively discriminate between Na⁺, K⁺, Cs⁺ or Li⁺ ions (FIG. 4E)

### Example 11—Na_{X} is Inhibited by Extracellular Ca²⁺

We measured currents from Na_{X}-QTT and observed that Ca²⁺ does not permeate (FIG. 5A). However, extracellular Ca²⁺ showed an inhibitory effect, where a physiologically-relevant concentration of Ca²⁺ (1 mM) inhibited ˜70% of the outward Na⁺ current (FIG. 5A). Notably, extracellular Ca²⁺ block is ˜10-fold more potent on Na_{X} relative to traditional Na_{V} channels, confirming physiochemical distinctions within the selectivity filter (FIGS. 4A and 4B). The divalent cations Zn²⁺ and Co²⁺ inhibited Na_{X}-QTT currents at 1 mM, whereas inhibition by the trivalent cation Gd³⁺ was more potent (FIG. 5B), demonstrating that pore block by multivalent cations is not substantially impacted by the wider and more electronegative selectivity filter structure of Na_{X} (FIG. 4B).

### Example 12—Na_{X} is Sensitive to Classic Na_{V} Channel Blockers

We tested a range of classical Na_{V} channel blockers to establish the pharmacological properties of Na_{X}-QTT. Remarkably, and strongly contrasting prior reports that Na_{X} is a TTX-resistant channel, tetrodotoxin showed inhibitory effects on Na_{X}-QTT at concentrations as low as 10 nM (FIG. 5C). The potency of TTX block suggests that it occurs at the selectivity filter, consistent with substantial conservation of side-chains implicated in TTX-block of sensitive Na_{V} channel subtypes (FIGS. 9 and 15A).

The general anaesthetic lidocaine was effective at blocking Na_{X}-QTT currents at low mM concentrations (FIG. 5C), presumably by binding within the central cavity of the channel. Other classical pore-blocking drugs like quinidine and loperamide were equally potent inhibitors of Na_{X}-QTT currents at high μM concentrations (FIG. 5C). By contrast, strong inhibitory effects were not observed for flecainide, ranolazine or phenytoin on Na_{X}-QTT currents at concentrations up to 300 μM (FIG. 5C).

### Example 13—Na_{X} Reveals Atypical Voltage Sensor-Like Domains

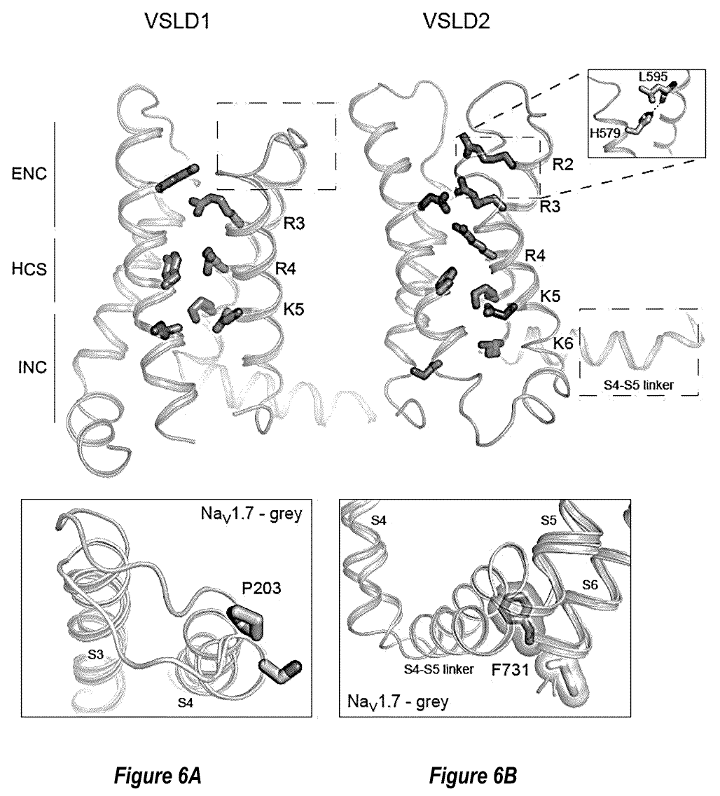

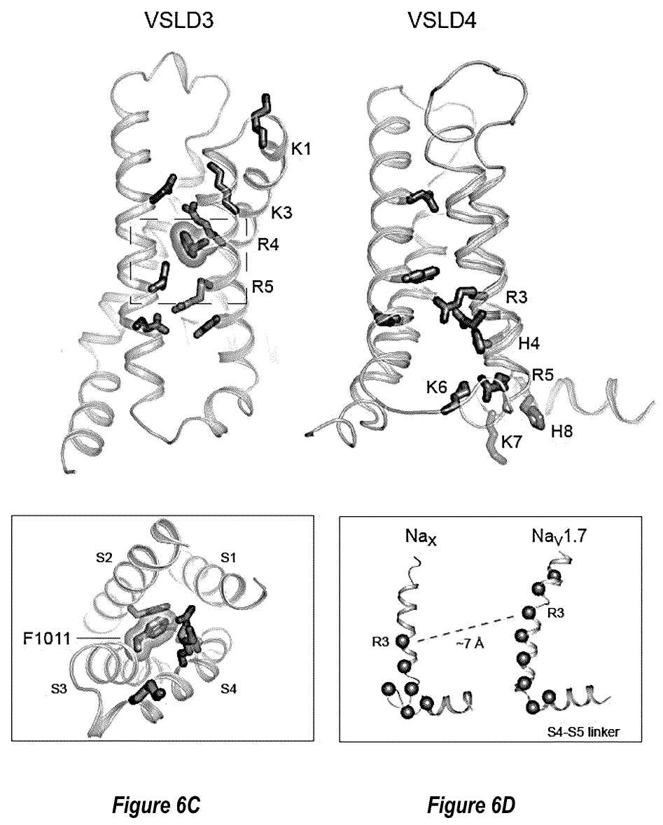

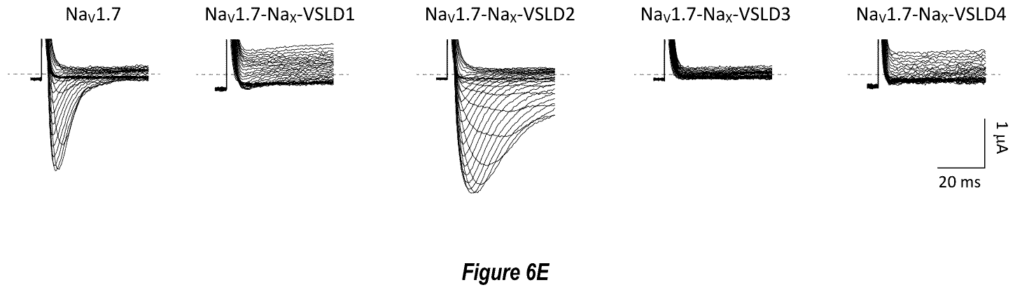

Conditions under which murine or human Na_{X} channels produce voltage-activated currents have not yet been identified, and this apparent lack of voltage sensitivity remains a puzzle. Paradoxically, the number of S4-gating charges is only modestly reduced in human Na_{X}^{14,15,22} (FIG. 9), and its VSLDs shares a high overall structural similarity with the VSDs of Na_{V} and Ca_{V} channels (FIGS. 13D and 13E). Intriguingly, while VSLD1, VSLD2 and VSLD3 in Na_{X} are all found in activated conformations, VSLD4 is deactivated (FIG. 6A-D). Closer evaluation of the Na_{X} VSLDs should provide insight into its apparent defective voltage-sensitivity.

Relative to human Na_{V} channels, VSLD1 in Na_{X} contains only three of four expected S4-gating charges (₂₀₃PTLQTARTLRILKIIP₂₁₈; SEQ ID NO: 87; FIGS. 6A and 9). K4 is coordinated by the intracellular negative charge cluster (INC) beneath the hydrophobic construction site (HCS), akin to an activated conformation (FIG. 6A)^{37,38}. Notably, glutamine neutralization at R1 is known to reduce voltage-sensitivity in other channels⁴⁰, and Pro203 imparts a radial bend within the extracellular S3-S4 loop that may impact S4 movement in Na_{X}, at a position conserved as serine in Na_{V} channels (FIGS. 6A and 9).

Na_{X} VSLD2 contains all five anticipated S4-gating charges (₅₉₃ALLRLFRMLRIFKLGK₆₀₈; SEQ ID NO: 88), and its activated state appears stabilized by a unique S3 His579 side-chain that bonds to the S4 carbonyl backbone (FIGS. 6B and 9). Furthermore, a radical shift of the DII S4-S5 linker towards the cytosol (3-7 Å) relative to human Na_{V} channel structures likely alters VSLD2 coupling (FIGS. 6B and 13D). This is propagated by a single residue deletion in the Na_{X} DII-S6 helix, which repositions Phe731 to undergo a ˜180° reorientation to enforce S4-S5 linker displacement (FIGS. 6B and 9).

Na_{X} VSLD3 displays only four of six canonical S4-gating charges (₁₀₂₄KPLISMKFLRPLRVLSQ₁₀₄₀; SEQ ID NO: 89), an alteration predicted to stabilize its activated conformation and decrease intrinsic voltage-sensitivity (FIGS. 6C and 9). Additionally, Phe1011 (S3) substantially and uniquely extends the HCS in VSLD3 (FIGS. 6C and 9), which would be expected to further impact S4-gating charge movement.

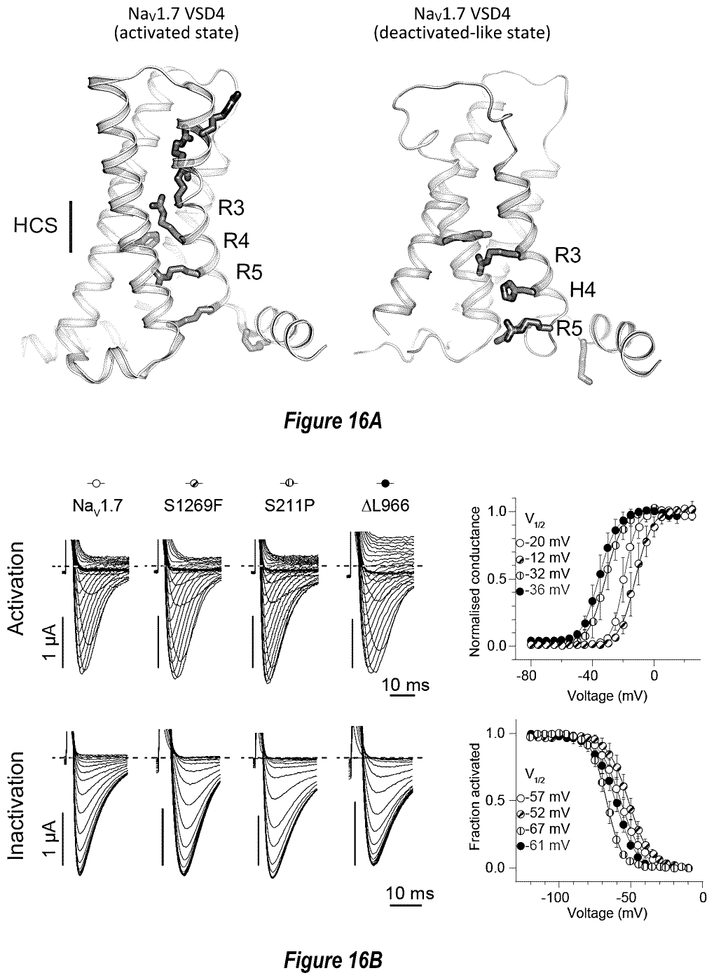

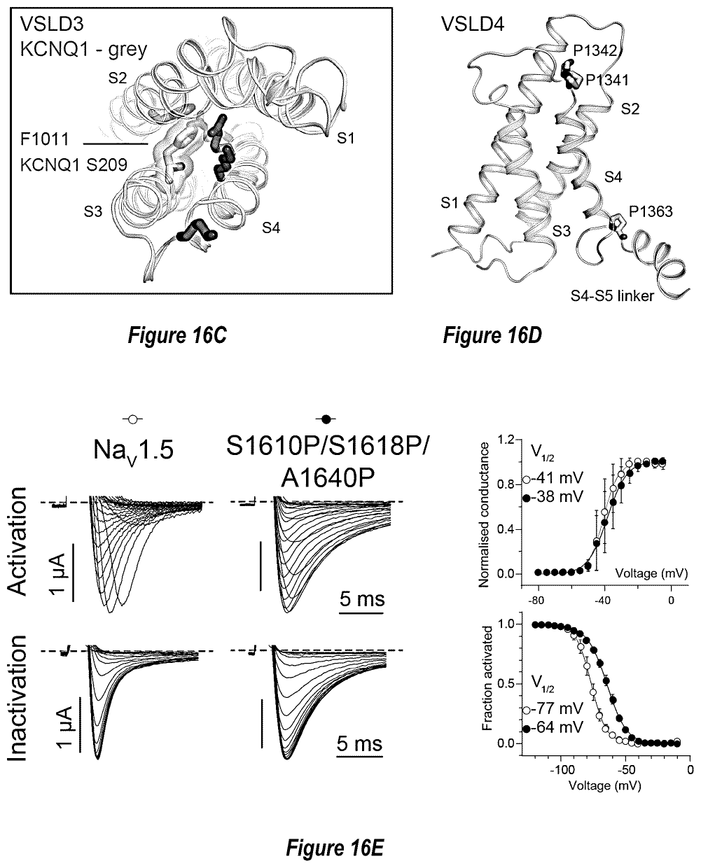

Na_{X} VSLD4 contains only four S4 gating charges (₁₃₄₆QLILLSRIIHMLRLGKGPKVFH₁₃₆₇; SEQ ID NO: 90), since H4 and H8 are unlikely to be charged at physiological pH^{37,39,44,45} (FIGS. 6D and 9). By comparison, VSD4 and the associated DIV S4-S5 linker in canonical Na_{V} channels conserve eight S4-gating charges that are inextricably linked to the fast inactivation mechanism. In stark contrast to human Na_{V} channel structures^{23,28,29,31,37} and expectations at 0 mV, VSLD4 of Na_{X} exists in a deactivated-like state, as indicated by a relative ˜7 Å displacement of the S4 towards the cytosol (FIGS. 6D and 16A). This observation is surprising, but consistent with the deficient voltage-sensitivity so far described for Na_{X}.

### Example 14—Probing Function of the Divergent Na_{X} VSLDs

Structural features identified in the VSLDs of Na_{X} suggest non-canonical contributions to channel function. Moreover, a number of prominent sequence peculiarities occur at positions in Na_{X} that have been reported as disease-causing alterations in related human channels. Pro203 in Na_{X} VSLD1 is equivalent to the pathogenic Ser211Pro mutation in Na_{V}1.7 reported to hyperpolarize channel activation by −12 mV (FIGS. 6A and 16B). Structure-based sequence alignments reveal that an analogous in-frame DII-S6 deletion is pathogenic in Na_{V}1.7 (ΔLeu966) and produces a −16 mV hyperpolarization of activation (FIGS. 6B and 16B). Phe1011 in Na_{X} VSLD3 corresponds to the pathogenic Ser209Phe mutation in KCNQ1 reported to impart a strong hyperpolarization (−45 mV) on channel activation (FIG. 16C) 43, while the equivalent Ser1269Phe substitution in Na_{V}1.7 produced a ˜8 mV depolarizing shift (FIGS. 6C and 16B). In addition, three prolines are uniquely present in VSLD4 of Na_{X} (FIGS. 9 and 16D), and mutation of the corresponding VSD4 residues in Na_{V}1.5 to proline imparted a strong depolarizing shift (15 mV) onto steady-state inactivation, consistent with the deactivated conformation observed in VSLD4 (FIGS. 6D, 16A and 16E).

To further probe Na_{X} VSLD function, we engineered chimeric constructs through targeted transfer of individual VSLDs into Na_{V}1.7. Remarkably, robust currents were recorded from injected Xenopus laevis oocytes expressing the Na_{V}1.7-Na_{X}-VSLD2 channel chimera (FIG. 6E), indicating that VSLD2 is either constitutively activated or intrinsically capable of electromechanical-coupling. By contrast, wholesale transfer of Na_{X}-VSLD1, VSLD3 or VSLD4 rendered Na_{V}1.7 chimeric channels non-functional (FIG. 6E). Thus, despite Na_{X} sharing global structural similarities with Na_{V}, Ca_{V} and Kv channels, our findings suggest that cumulative sequence adaptations may result in highly divergent functional outcomes across the channel superfamily.

### Example 15—Exemplary Na_{X}/Na_{V}1.7 Chimeric Constructs

A series of chimeric human Na_{X} proteins were constructed in which one or more regions from human Na_{V}1.7 were inserted in place of the corresponding Na_{X} sequences, as noted in Table 4 below. A construct is considered as functional (see Table 4) if the current amplitude of Xenopus laevis oocytes expressing the construct elicited at +80 mV or −100 mV from a holding potential of 0 mV is significantly higher than that from oocytes expressing human Na_{X} wild-type (see FIG. 18). Data for the first 7 chimeras of Table 4, and for a hNa_{X}-hNa_{V}1.7 DIV chimera, are also described in Example 6 and are shown in FIG. 2E.

TABLE 4
Constructs

| SEQ | Construct | | |
|---|---|---|---|
| ID | No. | | |
| NO | (FIG. 18) | Construct | Function* |
| 2 | 1 | hNaₓ_{_{—}}hNaᵥ1.7_DI-DII chimera | No |
| 3 | 2 | hNaₓ_{_{—}}hNaᵥ1.7_DIII-DIV chimera | Yes |
| 4 | 3 | hNaₓ_{_{—}}hNaᵥ1.7_DII-DIII chimera | Yes |
| 5 | 4 | hNaₓ_{_{—}}hNaᵥ1.7_DI-DIV chimera | No |
| 6 | 5 | hNaₓ-hNaᵥ1.7_DI chimera | No |
| 7 | 6 | hNaₓ-hNaᵥ1.7_DII chimera | No |
| 8 | 7 | hNaₓ-hNaᵥ1.7_DIII chimera | Yes |
| 9 | 8 | hNaₓ-hNaᵥ1.7_DIV-DIII-DIVlinker chimera | No |
| 10 | 9 | hNaₓ_{_{—}}hNaᵥ1.7_DIII_Chimera_SF chimera | Yes |
| 11 | 10 | hNaₓ_{_{—}}hNaᵥ1.7_D3-D4link chimera | No |
| 12 | 11 | hNaₓ_{_{—}}hNaᵥ1.7_D3-D4link-PM chimera | Yes |
| 13 | 12 | hNaₓ_{_{—}}hNaᵥ1.7_D3-D4link-PM-S4S5link chimera | No |
| 14 | 13 | hNaₓ_{_{—}}hNaᵥ1.7_VSD3 chimera | No |
| 15 | 14 | hNaₓ_{_{—}}hNaᵥ1.7_VSD3-S4S5link chimera | No |
| 16 | 15 | hNaₓ_{_{—}}hNaᵥ1.7_VSD3-S4S5link-PM chimera | Yes |
| 17 | 16 | hNaₓ_{_{—}}hNaᵥ1.7_DIII_VSD3-S3-S4 chimera | No |
| 18 | 17 | hNaₓ_{_{—}}hNaᵥ1.7_DIII_VSD3-S5-S6 chimera | No |
| 19 | 18 | hNaₓ_{_{—}}hNaᵥ1.7_DIII_VSD3-S3-S4loop_S4-S5link_S5 chimera | No |
| 20 | 19 | hNaₓ_{_{—}}hNaᵥ1.7_DIII_DIVS6-CTD chimera | Yes |
| 21 | 20 | Naₓ_{_{—}}Naᵥ1.7_D1-D2_linker chimera | No |
| 22 | 21 | Naₓ_{_{—}}Naᵥ1.7_D2-D3_linker chimera | No |
| 23 | 22 | Naₓ_{_{—}}Naᵥ1.7_D3-D4_linker chimera | No |
| 24 | 23 | Naₓ_{_{—}}Naᵥ1.7_ultimateloop chimera | No |
| 25 | 24 | Naₓ F724Q/I1189T/I1492T (QTT) | Yes |
| 26 | 25 | Naₓ L390E/I1189E/I1492E (EEE) | Yes |

### Discussion

The above Examples pursue an integrated analysis of structure, function and pharmacology to provide insight into the Na_{X} channel. Through comprehensive assessment of different expression systems, cofactors, and pharmacological activators, we demonstrated that human Na_{X} does not function as a voltage-activated channel. The molecular features responsible for the altered voltage sensitivity of Na_{X} might include single residue changes within the selectivity filter, voltage sensors, and pore module, as analogous changes can drastically impact the gating properties of Na_{V}1.5 and Na_{V}1.7 channels (FIGS. 6A-E, 9, 16B and 16E). In fact, many positional changes which impart structural alterations in Na_{X} appear to underlie disease hotspot positions in Na_{V} channels, but the significance of these observations remains unknown.

Using structure-guided protein engineering, we found that the transfer of entire homologous domains or subdomains (e.g. VSLDs) between Na_{X} and Na_{V}1.7 produces either non-functional constructs or channels with highly unusual gating characteristics (FIGS. 2E and 6E). These results suggest that the coupling or energetics of Na_{X} gating has diverged substantially from Na_{V} channels, which likely points to structural differences that burden the coupling interfaces between these closely related channel scaffolds. Notably, our studies do not exclude the possibility that Na_{X} may function as a voltage-modulated channel in specific cellular contexts. It is conceivable that binding of a putative auxiliary subunit or cofactor may impart some degree of voltage sensitivity onto Na_{X}, as recently described for the NALCN or TMEM16A channels.

### Reconciling Differences Over Na⁺-Sensing

Murine Na_{X} has been reported to produce non-inactivating inward currents upon increasing the extracellular Na⁺ concentration >150 mM. We only observed qualitatively similar recordings with low seal resistance and failed to reconstitute Na⁺-activated currents upon heterologous expression of human Na_{X}, even when co-expressed with factors expected to stabilize it on the plasma membrane (i.e. SAP97) (FIGS. 1D and 7F). It is plausible that sequence divergence or cellular context differences between murine and human Na_{X} proteins account for these seemingly conflicting functional outcomes (FIG. 9).

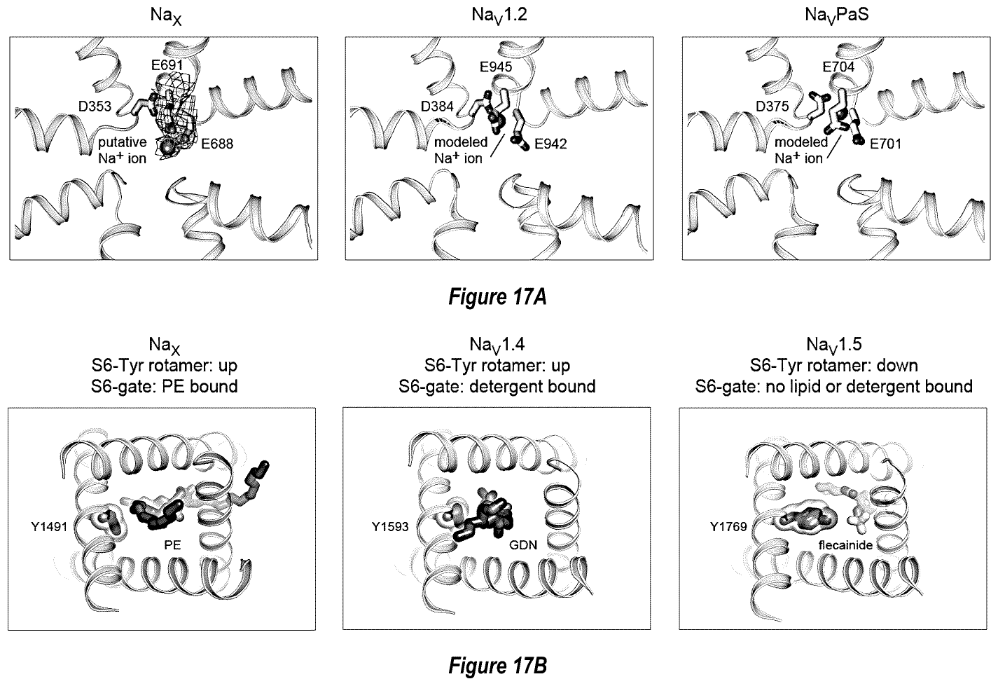

Our cryo-EM samples of β3-Na_{X} were prepared above the reported threshold required for Na⁺-dependent gating, in 200 mM NaCl⁹⁻¹¹. No clear Na⁺-sensing locus or activation-based mechanism was observed upon structural analyses, and the only map feature that we might assign as an extracellular Na⁺ ion is bound near the Na_{X} selectivity filter to a site also found in Na_{V} channels (FIG. 17A)^{29,49}. We infer that the Na⁺-sensing mechanism described for Na_{X} is most likely contributed by an exogenous protein factor or pathway, such as the Na⁺/K⁺ ATPase, the endothelin receptor, or a yet to be identified system. It is conceivable that post-translational modification of the predicted phosphorylation sites along the Na_{X} DIII-DIV linker (FIG. 14B) might help dislodge the IFI motif from its pore module receptor site to facilitate S6-dilation and channel gating. Alternatively, the presence of unusual but highly conserved histidine residues found within DIV and VSLD4 of Na_{X} could also raise speculation of a potential intracellular divalent cation- or pH-sensitive activation or modulation mechanism (FIGS. 6D and 9).

The Na_{X} Hypothesis: A Non-Selective Na⁺ Leak Channel

Guided by structural analyses and reiterative protein-engineering experiments, we found that targeted mutation of three residues around the hydrophobic S6-gate region is sufficient to produce robust ionic currents through human Na_{X} expressed in Xenopus laevis oocytes and HEK293T cells (FIGS. 3, 4D, 4E and 5). Presumably, the introduction of three polar side-chains into the Na_{X}-QTT channel construct will hydrate the S6-gate, dislodge bound membrane lipids, and allow for pore dilation and ion conduction (FIGS. 2B-2D). This conceptual and technical breakthrough allowed us to characterize the basic properties of human Na_{X} for the first time.

Distinct from Na_{V} channels, Na_{X}-QTT effectively conducts monovalent cations as an Ohmic leak channel in the absence of extracellular Ca²⁺ ions (FIGS. 3, 4D, 4E). Ca²⁺ does not permeate but blocks Na_{X}-QTT, so in the presence of physiological levels of extracellular Ca²⁺, Na_{X}-QTT currents are outward-rectifying with only a small inward leak component at negative membrane potentials (FIG. 5A). This unique current phenotype has pharmacological sensitivities expected for a Na_{V}-like channel, including apparent selectivity filter block by TTX, Zn²⁺, Co²⁺ and Gd³⁺, and presumed pore block by lidocaine, quinidine and loperamide (FIGS. 5B and 5C). Flecainide, ranolazine or phenytoin did not produce strong inhibition of Na_{X}-QTT currents (FIG. 5C), indicating a distinctive pharmacological profile of human Na_{X} relative to canonical Na_{V} channels, and possibly reflecting unique central cavity-lining residues like DIV-Trp1484 (FIG. 13F), or the potential absence of an inactivated state in Na_{X} (FIGS. 3B and 4D). Although Na_{X}-QTT currents were non-inactivating and not modulated by voltage, future studies will be required to define these important characteristics in wild-type human Na_{X} channels because the pore-wetting QTT-mutations are targeted to the S6-gate region. Nevertheless, it is noteworthy that an earlier study had suggested Na_{X} does function as a leak channel in vivo.

The physical, chemical and electrostatic profile of the Na_{X} selectivity filter structure meets expectations of a cation-selective channel (FIGS. 4A and 4B), but it lacks the DIII-lysine side-chain that imparts Na⁺-selectivity in Na_{V} channels (FIG. 9). Indeed, our electrophysiological characterization of Na_{X}-QTT revealed no selectivity among monovalent cations examined (Na⁺, K⁺, Cs⁺ and Li⁺) (FIG. 4E). Therefore, we hypothesize that human Na_{X} functions as Na⁺-leak channel which is sensitive to modulation by extracellular Ca²⁺ under physiological conditions (FIG. 5A). Notably, Ca²⁺ block of Na_{X}-QTT is ˜10-fold more potent than Ca²⁺ block on Na_{V} channels (FIG. 5A), but similar to the Ca²⁺ block sensitivity of the distantly related non-selective NALCN Na⁺-leak channel, perhaps reflecting a shared control mechanism to avoid excessive depolarizing Na⁺ influx into cells.

### Visualizing Pore-Bound Membrane Lipids

A striking feature of our Na_{X} channel structures determined in detergent or lipid nanodiscs was the visualization of a non-conductive state where lipids completely occlude the ion conduction pathway and seal the S6-gate (FIGS. 2C, 2D and 14F). The phospholipid which plugs the S6-gate is reminiscent of cholesterol-like molecules bound at the S6-gate in Na_{V} channel structures^{28,29,31} (FIG. 17B). Occupancy of these hydrophobic ligands at the S6-gate appears to correlate with the “up” conformation of a conserved DIV-S6 tyrosine side-chain that has been classically implicated in drug block in Na_{V} channels (FIGS. 9 and 17B)^{23,31-33}. Notably, drug binding within the central cavity can enforce a “down” conformation of this conserved S6 tyrosine^{30,31}, concurrent with displacement of the lipid-like density at the S6-gate (FIG. 17B)³¹. Our Na_{X} structures therefore support an emerging paradigm whereby membrane lipids can seal the S6-gate, and that lipid occupancy may modulate ion conductance, drug binding and pore structure. This developing model strongly contrasts with the long-held view that the S6-gate must close tightly and completely shut in Na_{V} channels³⁰.

### Summary and Outlook

The basic physiological and pharmacological characteristics of the Na_{X} channel have remained mysterious since its cloning nearly three decades ago. We find that human Na_{X} does not function independently as a Na⁺-activated channel, nor as a voltage-activated channel, under the conditions that we have examined. Through structure-guided protein engineering efforts, we uncovered an approach to characterize a human Na_{X} channel variant using traditional heterologous expression systems. The Na_{X}-QTT channel construct displayed a distinctive but Na_{V} channel-like pharmacological profile, including potent inhibition by Ca²⁺ and TTX, and revealed a non-selective cationic conductance suggesting that Na_{X} channel may operate as a Ca²⁺-modulated, Na⁺ leak channel in vivo. Overall, this study provides essential tools to further interrogate the physiology and pharmacology of Na_{X}, and greatly advances our understanding of this atypical ion channel.

### REFERENCES

1 Noda, M. & Hiyama, T. Y. Sodium sensing in the brain. Pflugers Arch 467, 465-474, doi:10.1007/s00424-014-1662-4 (2015).
2 Andersson, B. Thirst—and brain control of water balance. Am Sci 59, 408-415 (1971).
3 Liamis, G. et al. Electrolyte disorders in community subjects: prevalence and risk factors. Am J Med 126, 256-263, doi:10.1016/j.amjmed.2012.06.037 (2013).
4 Adrogue, H. J. Consequences of inadequate management of hyponatremia. Am J Nephrol 25, 240-249, doi:10.1159/000086019 (2005).
5 Watanabe, E. et al. Nav2/NaG channel is involved in control of salt-intake behavior in the CNS. J Neurosci 20, 7743-7751 (2000).
6 Nomura, K. et al. [Na(+)] Increases in Body Fluids Sensed by Central NaX Induce Sympathetically Mediated Blood Pressure Elevations via H(+)-Dependent Activation of ASIC1a. Neuron 101, 60-75 e66, doi:10.1016/j.neuron.2018.11.017 (2019).
7 Hiyama, T. Y. et al. Autoimmunity to the sodium-level sensor in the brain causes essential hypernatremia. Neuron 66, 508-522, doi:10.1016/j.neuron.2010.04.017 (2010).
8 Xu, W. et al. Sodium channel NaX is a regulator in epithelial sodium homeostasis. Sci Transl Med 7, 312ra177, doi:10.1126/scitranslmed.aad0286 (2015).
9 Hiyama, T. Y. et al. Na(x) channel involved in CNS sodium-level sensing. Nat Neurosci 5, 511-512, doi:10.1038/nn0602-856 (2002).
10 Shimizu, H. et al. Glial NaX channels control lactate signaling to neurons for brain [Na+] sensing. Neuron 54, 59-72, doi:10.1016/j.neuron.2007.03.014 (2007).
11 Hiyama, T. Y. et al. Endothelin-3 expression in the subfornical organ enhances the sensitivity of Na(x), the brain sodium-level sensor, to suppress salt intake. Cell Metab 17, 507-519, doi:10.1016/j.cmet.2013.02.018 (2013).
12 Matsumoto, M. et al. Channel properties of NaX expressed in neurons. PLOS One 10, e0126109, doi:10.1371/journal.pone.0126109 (2015).
13 Noda, M. & Hiyama, T. Y. The Na(x) Channel: What It Is and What It Does. Neuroscientist 21, 399-412, doi:10.1177/1073858414541009 (2015).
14 Akopian, A. N., Souslova, V., Sivilotti, L. & Wood, J. N. Structure and distribution of a broadly expressed atypical sodium channel. FEBS Lett 400, 183-187, doi:10.1016/s0014-5793 (96) 01389-0 (1997).
15 Felipe, A., Knittle, T. J., Doyle, K. L. & Tamkun, M. M. Primary structure and differential expression during development and pregnancy of a novel voltage-gated sodium channel in the mouse. J Biol Chem 269, 30125-30131 (1994).
16 Goldin, A. L. et al. Nomenclature of voltage-gated sodium channels. Neuron 28, 365-368, doi:10.1016/s0896-6273 (00) 00116-1 (2000).
17 Noda, M. et al. Existence of distinct sodium channel messenger RNAs in rat brain. Nature 320, 188-192, doi:10.1038/320188a0 (1986).
18 Shen, H. et al. Structure of a eukaryotic voltage-gated sodium channel at near-atomic resolution. Science 355, doi:10.1126/science.aal4326 (2017).
19 Chanda, B. & Bezanilla, F. Tracking voltage-dependent conformational changes in skeletal muscle sodium channel during activation. J Gen Physiol 120, 629-645, doi:10.1085/jgp.20028679 (2002).
20 Capes, D. L., Goldschen-Ohm, M. P., Arcisio-Miranda, M., Bezanilla, F. & Chanda, B. Domain IV voltage-sensor movement is both sufficient and rate limiting for fast inactivation in sodium channels. J Gen Physiol 142, 101-112, doi:10.1085/jgp.201310998 (2013).
21 Clairfeuille, T. et al. Structural basis of alpha-scorpion toxin action on NaV channels. Science 363, doi:10.1126/science.aav8573 (2019).
22 George, A. L., Jr., Knittle, T. J. & Tamkun, M. M. Molecular cloning of an atypical voltage-gated sodium channel expressed in human heart and uterus: evidence for a distinct gene family. Proc Natl Acad Sci USA 89, 4893-4897, doi:10.1073/pnas.89.11.4893 (1992).
23 Shen, H., Liu, D., Wu, K., Lei, J. & Yan, N. Structures of human Nav1.7 channel in complex with auxiliary subunits and animal toxins. Science 363, 1303-1308, doi:10.1126/science.aaw2493 (2019).
24 Laedermann, C. J., Syam, N., Pertin, M., Decosterd, I. & Abriel, H. beta1- and beta3-voltage-gated sodium channel subunits modulate cell surface expression and glycosylation of Nav1.7 in HEK293 cells. Front Cell Neurosci 7, 137, doi:10.3389/fncel.2013.00137 (2013).
25 Kanellopoulos, A. H. et al. Mapping protein interactions of sodium channel NaV1.7 using epitope-tagged gene-targeted mice. EMBO J 37, 427-445, doi:10.15252/embj.201796692 (2018).
26 Sokolov, M. V. et al. Co-expression of beta Subunits with the Voltage-Gated Sodium Channel NaV1.7: the Importance of Subunit Association and Phosphorylation and Their Effects on Channel Pharmacology and Biophysics. J Mol Neurosci 65, 154-166, doi:10.1007/s12031-018-1082-6 (2018).
27 Das, S., Gilchrist, J., Bosmans, F. & Van Petegem, F. Binary architecture of the Nav1.2-beta2 signaling complex. Elife 5, doi:10.7554/eLife.10960 (2016).
28 Pan, X. et al. Structure of the human voltage-gated sodium channel Nav1.4 in complex with beta1. Science 362, doi:10.1126/science.aau2486 (2018).
29 Pan, X. et al. Molecular basis for pore blockade of human Na (+) channel Nav1.2 by the mu-conotoxin KIIIA. Science 363, 1309-1313, doi:10.1126/science.aaw2999 (2019).
30 Jiang, D. et al. Structure of the Cardiac Sodium Channel. Cell 180, 122-134 e110, doi:10.1016/j.cell.2019.11.041 (2020).
31 Li, Z. et al. Structural basis for pore blockade of the human cardiac sodium channel Nav1.5 by tetrodotoxin and quinidine. bioRxiv doi.org/10.1101/2019.12.30.890681 (2020).
32 Ragsdale, D. S., McPhee, J. C., Scheuer, T. & Catterall, W. A. Molecular determinants of state-dependent block of Na+ channels by local anesthetics. Science 265, 1724-1728, doi:10.1126/science.8085162 (1994).
33 Pless, S. A., Galpin, J. D., Frankel, A. & Ahern, C. A. Molecular basis for class Ib anti-arrhythmic inhibition of cardiac sodium channels. Nat Commun 2, 351, doi:10.1038/ncomms1351 (2011).
34 Hille, B. Local anesthetics: hydrophilic and hydrophobic pathways for the drug-receptor reaction. J Gen Physiol 69, 497-515, doi:10.1085/jgp.69.4.497 (1977).
35 Payandeh, J., Scheuer, T., Zheng, N. & Catterall, W. A. The crystal structure of a voltage-gated sodium channel. Nature 475, 353-358, doi:10.1038/nature10238 (2011).
36 Payandeh, J., Gamal El-Din, T. M., Scheuer, T., Zheng, N. & Catterall, W. A. Crystal structure of a voltage-gated sodium channel in two potentially inactivated states. Nature 486, 135-139, doi:10.1038/nature11077 (2012).
37 Ahuja, S. et al. Structural basis of Nav1.7 inhibition by an isoform-selective small-molecule antagonist. Science 350, aac5464, doi:10.1126/science.aac5464 (2015).
38 Tao, X., Lee, A., Limapichat, W., Dougherty, D. A. & Mackinnon, R. A gating charge transfer center in voltage sensors. Science 328, 67-73, doi:10.1126/science. 1185954 (2010).
39 Gagnon, D. G. & Bezanilla, F. A single charged voltage sensor is capable of gating the Shaker K⁺ channel. J Gen Physiol 133, 467-483, doi:10.1085/jgp.200810082 (2009).
40 Kontis, K. J., Rounaghi, A. & Goldin, A. L. Sodium channel activation gating is affected by substitutions of voltage sensor positive charges in all four domains. J Gen Physiol 110, 391-401, doi:10.1085/jgp.110.4.391 (1997).
41 Estacion, M. et al. Can robots patch-clamp as well as humans? Characterization of a novel sodium channel mutation. J Physiol 588, 1915-1927, doi:10.1113/jphysiol.2009.186114 (2010).
42 Yang, Y., Estacion, M., Dib-Hajj, S. D. & Waxman, S. G. Molecular architecture of a sodium channel S6 helix: radial tuning of the voltage-gated sodium channel 1.7 activation gate. J Biol Chem 288, 13741-13747, doi:10.1074/jbc.M113.462366 (2013).
43 Eldstrom, J. et al. Mechanistic basis for LQT1 caused by S3 mutations in the KCNQ1 subunit of IKs. J Gen Physiol 135, 433-448, doi:10.1085/jgp.200910351 (2010).
44 Kuhn, F. J. & Greeff, N. G. Movement of voltage sensor S4 in domain 4 is tightly coupled to sodium channel fast inactivation and gating charge immobilization. J Gen Physiol 114, 167-183, doi:10.1085/jgp.114.2.167 (1999).
45 Arnold, W. D. et al. Defective fast inactivation recovery of NaV 1.4 in congenital myasthenic syndrome. Ann Neurol 77, 840-850, doi:10.1002/ana.24389 (2015).
46 Bosmans, F., Martin-Eauclaire, M. F. & Swartz, K. J. Deconstructing voltage sensor function and pharmacology in sodium channels. Nature 456, 202-208, doi:10.1038/nature07473 (2008).
47 Bosmans, F., Puopolo, M., Martin-Eauclaire, M. F., Bean, B. P. & Swartz, K. J. Functional properties and toxin pharmacology of a dorsal root ganglion sodium channel viewed through its voltage sensors. J Gen Physiol 138, 59-72, doi:10.1085/jgp.201110614 (2011).
48 Xu, H. et al. Structural Basis of Nav1.7 Inhibition by a Gating-Modifier Spider Toxin. Cell 176, 702-715 e714, doi:10.1016/j.cell.2018.12.018 (2019).
49 Shen, H. et al. Structural basis for the modulation of voltage-gated sodium channels by animal toxins. Science 362, doi:10.1126/science.aau2596 (2018).
50 Mastronarde, D. N. Automated electron microscope tomography using robust prediction of specimen movements. J Struct Biol 152, 36-51, doi:10.1016/j.jsb.2005.07.007 (2005).
51 Tegunov, D. & Cramer, P. Real-time cryo-electron microscopy data preprocessing with Warp. Nat Methods 16, 1146-1152, doi:10.1038/s41592-019-0580-y (2019).
52 Zivanov, J. et al. New tools for automated high-resolution cryo-EM structure determination in RELION-3. Elife 7, doi:10.7554/eLife.42166 (2018).
53 Grant, T., Rohou, A. & Grigorieff, N. cisTEM, user-friendly software for single-particle image processing. Elife 7, doi:10.7554/eLife.35383 (2018).
54 Scheres, S. H. RELION: implementation of a Bayesian approach to cryo-EM structure determination. J Struct Biol 180, 519-530, doi:10.1016/j.jsb.2012.09.006 (2012).
55 Zheng, S. Q. et al. MotionCor2: anisotropic correction of beam-induced motion for improved cryo-electron microscopy. Nat Methods 14, 331-332, doi:10.1038/nmeth.4193 (2017).
56 Rohou, A. & Grigorieff, N. CTFFIND4: Fast and accurate defocus estimation from electron micrographs. J Struct Biol 192, 216-221, doi:10.1016/j.jsb.2015.08.008 (2015).
57 Matthies, D. et al. Single-particle cryo-EM structure of a voltage-activated potassium channel in lipid nanodiscs. Elife 7, doi:10.7554/eLife.37558 (2018).
58 Kelley, L. A., Mezulis, S., Yates, C. M., Wass, M. N. & Sternberg, M. J. The Phyre2 web portal for protein modeling, prediction and analysis. Nat Protoc 10, 845-858, doi:10.1038/nprot.2015.053 (2015).
59 Emsley, P., Lohkamp, B., Scott, W. G. & Cowtan, K. Features and development of Coot. Acta Crystallogr D Biol Crystallogr 66, 486-501, doi:10.1107/S0907444910007493 (2010).
60 Afonine, P. V. et al. Real-space refinement in PHENIX for cryo-EM and crystallography. Acta Crystallogr D Struct Biol 74, 531-544, doi:10.1107/S2059798318006551 (2018).
61 Goddard, T. D. et al. UCSF ChimeraX: Meeting modern challenges in visualization and analysis. Protein Sci 27, 14-25, doi:10.1002/pro.3235 (2018).
62 Croll, T. I. ISOLDE: a physically realistic environment for model building into low-resolution electron-density maps. Acta Crystallogr D Struct Biol 74, 519-530, doi:10.1107/S2059798318002425 (2018).
63 The PyMOL Molecular Graphics System, V. r. p., Schrödinger, LLC.
64 Pettersen, E. F. et al. UCSF Chimera--a visualization system for exploratory research and analysis. J Comput Chem 25, 1605-1612, doi:10.1002/jcc.20084 (2004).
65 Chovancova, E. et al. CAVER 3.0: a tool for the analysis of transport pathways in dynamic protein structures. PLOS Comput Biol 8, e1002708, doi:10.1371/journal.pcbi. 1002708 (2012).
66 Sievers, F. et al. Fast, scalable generation of high-quality protein multiple sequence alignments using Clustal Omega. Mol Syst Biol 7, 539, doi:10.1038/msb.2011.75 (2011).
67 Robert, X. & Gouet, P. Deciphering key features in protein structures with the new ENDscript server. Nucleic Acids Res 42, W320-324, doi:10.1093/nar/gku316 (2014).
68 Chua, C. H., Wulf, M., Weidling, C., Rasmussen, L. P. & Pless, S. A. The NALCN channel complex is voltage sensitive and directly modulated by extracellular calcium. Sci Adv 6, eaaz3154 (2020).

### SEQUENCE TABLE

The following table further describes certain sequences referenced herein.

TABLE 5
Sequences

| SEQ | | |
|---|---|---|
| ID NO. | Identity | Sequence |
| 1 | Wild-type human | MLASPEPKGLVPFTKESFELIKQHIAKTHNEDHEEEDLKPTPDLEVGKKLPF |
| | Nax (hNax) | IYGNLSQGMVSEPLEDVDPYYYKKKNTFIVLNKNRTIFRFNAASILCTLSPF |
| | (Uniprot Q01118- | NCIRRTTIKVLVHPFFQLFILISVLIDCVEMSLTNLPKWRPVLENTLLGIYT |
| | 1) | FEILVKLFARGVWAGSFSFLGDPWNWLDESVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVERILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLFNESMLWENAKMNFDNVGNGELSLLQVATENGW |
| | | ITIMNSAIDSVAVNIQPHFEVNIYMYCYFINFIIFGVFLPLSMLITVIIDNF |
| | | NKHKIKLGGSNIFITVKQRKQYRRLKKLMYEDSQRPVPRPLNKLQGFIFDVV |
| | | TSQAFNVIVMVLICFQAIAMMIDTDVQSLQMSIALYWINSIFVMLYTMECIL |
| | | KLIAFRCFYFTIAWNIFDFMVVIFSITGLCLPMTVGSYLVPPSLVQLILLSR |
| | | IIHMLRLGKGPKVFHNLMLPLMLSLPALLNIILLIFLVMFIYAVFGMYNFAY |
| | | VKKEAGINDVSNFETFGNSMLCLFQVAIFAGWDGMLDAIFNSKWSDCDPDKI |
| | | NPGTQVRGDCGNPSVGIFYFVSYILISWLIIVNMYIVVVMEFLNIASKKKNK |
| | | TLSEDDFRKFFQVWKRFDPDRTQYIDSSKLSDFAAALDPPLFMAKPNKGQLI |
| | | ALDLPMAVGDRIHCLDILLAFTKRVMGQDVRMEKVVSEIESGELLANPFKIT |
| | | CEPITTTLKRKQEAVSATIIQRAYKNYRLRRNDKNTSDIHMIDGDRDVHATK |
| | | EGAYFDKAKEKSPIQSQI |
| 2 | hNax_hNav1.7_DI | MAMLPPPGPQSFVHFTKQSLALIEQRIAERKSKEPKEEKKDDDEEAPKPSSD |
| | -DII_chimera | LEAGKQLPFIYGDIPPGMVSEPLEDLDPYYADKKTFIVLNKGKTIFRFNATP |
| | | ALYMLSPFSPLRRISIKILVHSLESMLIMCTILTNCIFMTMNNPPDWTKNVE |
| | | YTFTGIYTFESLVKILARGFCVGEFTFLRDPWNWLDFVVIVFAYLTEFVNLG |
| | | NVSALRTFRVLRALKTISVIPGLKTIVGALIQSVKKLSDVMILTVFCLSVFA |
| | | LIGLQLFMGNLKHKCFRNSLENNETLESIMNTLESEEDFRKYFYYLEGSKDA |
| | | LLCGFSTDSGQCPEGYTCVKIGRNPDYGYTSFDTFSWAFLALFRLMTQDYWE |
| | | NLYQQTLRAAGKTYMIFFVVVIFLGSFYLINLILAVVAMAYEEQNQANIEEA |
| | | KQKELEFQQMLDRLKKEQEEAEAIAAAAAEYTSIRRSRIMGLSESSSETSKL |
| | | SSKSAKERRNRRKKKNQKKLSSGEEKGDAEKLSKSESEDSIRRKSFHLGVEG |
| | | HRRAHEKRLSTPNQSPLSIRGSLFSARRSSRTSLFSFKGRGRDIGSETEFAD |
| | | DEHSIFGDNESRRGSLFVPHRPQERRSSNISQASRSPPMLPVNGKMHSAVDC |
| | | NGVVSLVDGRSALMLPNGQLLPEVIIDKATSDDSGTTNQIHKKRRCSSYLLS |
| | | EDMLNDPNLRQRAMSRASILTNTVEELEESRQKCPPWWYRFAHKFLIWNCSP |
| | | YWIKFKKCIYFIVMDPFVDLAITICIVLNTLFMAMEHHPMTEEFKNVLAIGN |
| | | LVFTGIFAAEMVLKLIAMDPYEYFQVGWNIFDSLIVTLSLVELFLADVEGLS |
| | | VLRSFRLLRVFKLAKSWPTLNMLIKIIGNSVGALGNLTLVLAIIVFIFAVVG |
| | | MQLFGKSYKECVCKINDDCTLPRWHMNDFFHSFLIVFRVLCGEWIETMWDCM |
| | | EVAGQAMCLIVYMMVMVIGNLVVLNLFLALLLSSFSSDNLTAIEEDPDANNL |
| | | QIAVTRIKKGINYVKQTLREFILKAFSKKPKISREIRQAEDLNTKKENYISN |
| | | HTLAEMSKGHNFLKEKDKISGFGSSVDKHLMEDSDGQSFIHNPSLTVTVPIA |
| | | PGESDLENMNAEELSSDSDSEYSKVRLNRSSSSECSTVDNPLPGEGEEAEAE |
| | | PMNSDEPEACFTDGCVWRFSCCQVNIESGKGKIWQNIRKTCCKIVENNWFKC |
| | | FIGLVTLLSTGTLAFEDIYMDQRKTIKILLEYADMIFTYIFILEMLLKWMAY |
| | | GFKAYFSNGWYRLDFVVVIVFCLSLIGKTREELKPLISMKFLRPLRVLSQFE |
| | | RMKVVVRALIKTTLPTLNVFLVCLMIWLIFSIMGVDLFAGRFYECIDPTSGE |
| | | RFPSSEVMNKSRCESLLFNESMLWENAKMNFDNVGNGFLSLLQVATENGWIT |
| | | IMNSAIDSVAVNIQPHFEVNIYMYCYFINFIIFGVFLPLSMLITVIIDNENK |
| | | HKIKLGGSNIFITVKQRKQYRRLKKLMYEDSQRPVPRPLNKLQGFIFDVVTS |
| | | QAFNVIVMVLICFQAIAMMIDTDVQSLQMSIALYWINSIFVMLYTMECILKL |
| | | IAFRCFYFTIAWNIFDFMVVIFSITGLCLPMTVGSYLVPPSLVQLILLSRII |
| | | HMLRLGKGPKVFHNLMLPLMLSLPALLNIILLIFLVMFIYAVFGMYNFAYVK |
| | | KEAGINDVSNFETFGNSMLCLFQVAIFAGWDGMLDAIFNSKWSDCDPDKINP |
| | | GTQVRGDCGNPSVGIFYFVSYILISWLIIVNMYIVVVMEFLNIASKKKNKTL |
| | | SEDDFRKFFQVWKRFDPDRTQYIDSSKLSDFAAALDPPLFMAKPNKGQLIAL |
| | | DLPMAVGDRIHCLDILLAFTKRVMGQDVRMEKVVSEIESGELLANPFKITCE |
| | | PITTTLKRKQEAVSATIIQRAYKNYRLRRNDKNTSDIHMIDGDRDVHATKEG |
| | | AYFDKAKEKSPIQSQI |
| 3 | hNax_hNav1.7_DI | MLASPEPKGLVPFTKESFELIKQHIAKTHNEDHEEEDLKPTPDLEVGKKLPF |
| | II-DIV_chimera | NCIRRTTIKVLVHPFFQLFILISVLIDCVEMSLTNLPKWRPVLENTLLGIYT |
| | | IYGNLSQGMVSEPLEDVDPYYYKKKNTFIVLNKNRTIFRENAASILCTLSPF |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFGWALFALFRLMAQDYPEVLYHQILY |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDELKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGQDIFMTEEQKKYYNAMKKLGSKKPQKPIPRPGNKIQG |
| | | CIFDLVTNQAFDISIMVLICLNMVTMMVEKEGQSQHMTEVLYWINVVFIILF |
| | | TGECVLKLISLRHYYFTVGWNIFDFVVVIISIVGMFLADLIETYFVSPTLER |
| | | VIRLARIGRILRLVKGAKGIRTLLFALMMSLPALFNIGLLLFLVMFIYAIFG |
| | | MSNFAYVKKEDGINDMFNFETFGNSMICLFQITTSAGWDGLLAPILNSKPPD |
| | | CDPKKVHPGSSVEGDCGNPSVGIFYFVSYIIISFLVVVNMYIAVILENFSVA |
| | | TEESTEPLSEDDFEMFYEVWEKFDPDATQFIEFSKLSDFAAALDPPLLIAKP |
| | | NKVQLIAMDLPMVSGDRIHCLDILFAFTKRVLGESGEMDSLRSQMEERFMSA |
| | | NPSKVSYEPITTTLKRKQEDVSATVIQRAYRRYRLRQNVKNISSIYIKDGDR |
| | | DDDLLNKKDMAFDNVNENSSPEKTDATSSTTSPPSYDSVTKPDKEKYEQDRT |
| | | EKEDKGKDSKESKK |
| 4 | hNax_hNav1.7_DI | MLASPEPKGLVPFTKESFELIKQHIAKTHNEDHEEEDLKPTPDLEVGKKLPF |
| | I-DIII_chimera | IYGNLSQGMVSEPLEDVDPYYYKKKNTFIVLNKNRTIFRFNAASILCTLSPF |
| | | NCIRRTTIKVLVHPFFQLFILISVLIDCVEMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPYWIKFKKCIYFIVMDPFVDLAITICIVLNTL |
| | | FMAMEHHPMTEEFKNVLAIGNLVFTGIFAAEMVLKLIAMDPYEYFQVGWNIE |
| | | DSLIVTLSLVELFLADVEGLSVLRSFRLLRVFKLAKSWPTLNMLIKIIGNSV |
| | | GALGNLTLVLAIIVFIFAVVGMQLFGKSYKECVCKINDDCTLPRWHMNDFFH |
| | | SFLIVFRVLCGEWIETMWDCMEVAGQAMCLIVYMMVMVIGNLVVLNLFLALL |
| | | LSSFSSDNLTAIEEDPDANNLQIAVTRIKKGINYVKQTLREFILKAFSKKPK |
| | | ISREIRQAEDLNTKKENYISNHTLAEMSKGHNFLKEKDKISGFGSSVDKHLM |
| | | EDSDGQSFIHNPSLTVTVPIAPGESDLENMNAEELSSDSDSEYSKVRLNRSS |
| | | SSECSTVDNPLPGEGEEAEAEPMNSDEPEACFTDGCVWRFSCCQVNIESGKG |
| | | KIWWNIRKTCYKIVEHSWFESFIVLMILLSSGALAFEDIYIERKKTIKIILE |
| | | YADKIFTYIFILEMLLKWIAYGYKTYFTNAWCWLDFLIVDVSLVTLVANTLG |
| | | YSDLGPIKSLRTLRALRPLRALSRFEGMRVVVNALIGAIPSIMNVLLVCLIF |
| | | WLIFSIMGVNLFAGKFYECINTTDGSRFPASQVPNRSECFALMNVSQNVRWK |
| | | NLKVNFDNVGLGYLSLLQVATFKGWTIIMYAAVDSVNVDKQPKYEYSLYMYI |
| | | YFVVFIIFGSFFTLNLFIGVIIDNFNQQKKKLGGQDIFMTEEQKKYYNAMKK |
| | | LGSKKPQKPIPRPGNKLQGFIFDVVTSQAFNVIVMVLICFQAIAMMIDTDVQ |
| | | SLQMSIALYWINSIFVMLYTMECILKLIAFRCFYFTIAWNIFDFMVVIFSIT |
| | | GLCLPMTVGSYLVPPSLVQLILLSRIIHMLRLGKGPKVFHNLMLPLMLSLPA |
| | | LLNIILLIFLVMFIYAVFGMYNFAYVKKEAGINDVSNFETFGNSMLCLFQVA |
| | | IFAGWDGMLDAIFNSKWSDCDPDKINPGTQVRGDCGNPSVGIFYFVSYILIS |
| | | WLIIVNMYIVVVMEFLNIASKKKNKTLSEDDFRKFFQVWKRFDPDRTQYIDS |
| | | SKLSDFAAALDPPLFMAKPNKGQLIALDLPMAVGDRIHCLDILLAFTKRVMG |
| | | QDVRMEKVVSEIESGFLLANPFKITCEPITTTLKRKQEAVSATIIQRAYKNY |
| | | RLRRNDKNTSDIHMIDGDRDVHATKEGAYFDKAKEKSPIQSQI |
| 5 | hNax_hNav1.7_DI | MAMLPPPGPQSFVHFTKQSLALIEQRIAERKSKEPKEEKKDDDEEAPKPSSD |
| | -DIV_chimera | LEAGKQLPFIYGDIPPGMVSEPLEDLDPYYADKKTFIVLNKGKTIFRENATP |
| | | ALYMLSPFSPLRRISIKILVHSLFSMLIMCTILTNCIFMTMNNPPDWTKNVE |
| | | YTFTGIYTFESLVKILARGFCVGEFTFLRDPWNWLDFVVIVFAYLTEFVNLG |
| | | NVSALRTFRVLRALKTISVIPGLKTIVGALIQSVKKLSDVMILTVFCLSVFA |
| | | LIGLQLFMGNLKHKCFRNSLENNETLESIMNTLESEEDFRKYFYYLEGSKDA |
| | | LLCGFSTDSGQCPEGYTCVKIGRNPDYGYTSFDTFSWAFLALFRLMTQDYWE |
| | | NLYQQTLRAAGKTYMIFFVVVIFLGSFYLINLILAVVAMAYEEQNQANIEEA |
| | | KQKELEFQQMLDRLKKEQEEAEAIAAAAAEYTSIRRSRIMGLSESSSETSKL |
| | | SSKSAKERRNRRKKKNQKKLSSGEEKGDAEKLSKSESEDSIRRKSFHLGVEG |
| | | HRRAHEKRLSTPNQSPLSIRGSLFSARRSSRTSLFSFKGRGRDIGSETEFAD |
| | | DEHSIFGDNESRRGSLFVPHRPQERRSSNISQASRSPPMLPVNGKMHSAVDC |
| | | NGVVSLVDGRSALMLPNGQLLPEVIIDKATSDDSGTTNQIHKKRRCSSYLLS |
| | | EDMLNDPNLRQRAMSRASILTNTVEELEESRQKCPPWWYRFAHKFLIWNCSP |
| | | CWLKLKEFVHRIIMAPFTDLFLIICIILNVCFLTLEHYPMSKQTNTLLNIGN |
| | | LVFIGIFTAEMIFKIIAMHPYGYFQVGWNIFDSMIVFHGLIELCLANVAGMA |
| | | LLRLFRMLRIFKLGKYWPTFQILMWSLSNSWVALKDLVLLLFTFIFFSAAFG |
| | | MKLFGKNYEEFVCHIDKDCQLPRWHMHDFFHSFLNVFRILCGEWVETLWDCM |
| | | EVAGQSWCIPFYLMVILIGNLLVLYLFLALVSSFSSCKDVTAEENNEAKNLQ |
| | | LAVARIKKGINYVLLKILCKTQNVPKDTMDHVNEVYVKEDISDHTLSELSNT |
| | | QDFLKDKEKSSGTEKNATENESQSLIPSPSVSETVPIASGESDIENLDNKEI |
| | | QSKSGDGGSKEKIKQSSSSECSTVDIAISEEEEMFYGGERSKHLKNGCRRGS |
| | | SLGQISGASKKGKIWQNIRKTCCKIVENNWFKCFIGLVTLLSTGTLAFEDIY |
| | | MDQRKTIKILLEYADMIFTYIFILEMLLKWMAYGFKAYFSNGWYRLDFVVVI |
| | | VFCLSLIGKTREELKPLISMKFLRPLRVLSQFERMKVVVRALIKTTLPTLNV |
| | | FLVCLMIWLIFSIMGVDLFAGRFYECIDPTSGERFPSSEVMNKSRCESLLEN |
| | | ESMLWENAKMNFDNVGNGFLSLLQVATENGWITIMNSAIDSVAVNIQPHFEV |
| | | NIYMYCYFINFIIFGVFLPLSMLITVIIDNENKHKIKLGGSNIFITVKQRKQ |
| | | YRRLKKLMYEDSQRPVPRPLNKIQGCIFDLVTNQAFDISIMVLICLNMVTMM |
| | | VEKEGQSQHMTEVLYWINVVFIILFTGECVLKLISLRHYYFTVGWNIFDFVV |
| | | VIISIVGMFLADLIETYFVSPTLERVIRLARIGRILRLVKGAKGIRTLLFAL |
| | | MMSLPALFNIGLLLFLVMFIYAIFGMSNFAYVKKEDGINDMENFETFGNSMI |
| | | CLFQITTSAGWDGLLAPILNSKPPDCDPKKVHPGSSVEGDCGNPSVGIFYFV |
| | | SYIIISFLVVVNMYIAVILENFSVATEESTEPLSEDDFEMFYEVWEKFDPDA |
| | | TQFIEFSKLSDFAAALDPPLLIAKPNKVQLIAMDLPMVSGDRIHCLDILFAF |
| | | TKRVLGESGEMDSLRSQMEERFMSANPSKVSYEPITTTLKRKQEDVSATVIQ |
| | | RAYRRYRLRQNVKNISSIYIKDGDRDDDLLNKKDMAFDNVNENSSPEKTDAT |
| | | SSTTSPPSYDSVTKPDKEKYEQDRTEKEDKGKDSKESKK |
| 6 | hNax- | MAMLPPPGPQSFVHFTKQSLALIEQRIAERKSKEPKEEKKDDDEEAPKPSSD |
| | hNav1.7_DI_ | LEAGKQLPFIYGDIPPGMVSEPLEDLDPYYADKKTFIVLNKGKTIFRENATP |
| | chimera | ALYMLSPFSPLRRISIKILVHSLFSMLIMCTILTNCIFMTMNNPPDWTKNVE |
| | | YTFTGIYTFESLVKILARGFCVGEFTFLRDPWNWLDFVVIVFAYLTEFVNLG |
| | | NVSALRTFRVLRALKTISVIPGLKTIVGALIQSVKKLSDVMILTVFCLSVFA |
| | | LIGLQLFMGNLKHKCFRNSLENNETLESIMNTLESEEDFRKYFYYLEGSKDA |
| | | LLCGFSTDSGQCPEGYTCVKIGRNPDYGYTSFDTFSWAFLALFRLMTQDYWE |
| | | NLYQQTLRAAGKTYMIFFVVVIFLGSFYLINLILAVVAMAYEEQNQANIEEA |
| | | KQKELEFQQMLDRLKKEQEEAEAIAAAAAEYTSIRRSRIMGLSESSSETSKL |
| | | SSKSAKERRNRRKKKNQKKLSSGEEKGDAEKLSKSESEDSIRRKSFHLGVEG |
| | | HRRAHEKRLSTPNQSPLSIRGSLFSARRSSRTSLFSFKGRGRDIGSETEFAD |
| | | DEHSIFGDNESRRGSLFVPHRPQERRSSNISQASRSPPMLPVNGKMHSAVDC |
| | | NGVVSLVDGRSALMLPNGQLLPEVIIDKATSDDSGTTNQIHKKRRCSSYLLS |
| | | EDMLNDPNLRQRAMSRASILTNTVEELEESRQKCPPWWYRFAHKFLIWNCSP |
| | | CWLKLKEFVHRIIMAPFTDLFLIICIILNVCFLTLEHYPMSKQTNTLLNIGN |
| | | LVFIGIFTAEMIFKIIAMHPYGYFQVGWNIFDSMIVFHGLIELCLANVAGMA |
| | | LLRLFRMLRIFKLGKYWPTFQILMWSLSNSWVALKDLVLLLFTFIFFSAAFG |
| | | MKLFGKNYEEFVCHIDKDCQLPRWHMHDFFHSELNVFRILCGEWVETLWDCM |
| | | EVAGQSWCIPFYLMVILIGNLLVLYLFLALVSSFSSCKDVTAEENNEAKNLQ |
| | | LAVARIKKGINYVLLKILCKTQNVPKDTMDHVNEVYVKEDISDHTLSELSNT |
| | | QDFLKDKEKSSGTEKNATENESQSLIPSPSVSETVPIASGESDIENLDNKEI |
| | | QSKSGDGGSKEKIKQSSSSECSTVDIAISEEEEMFYGGERSKHLKNGCRRGS |
| | | SLGQISGASKKGKIWQNIRKTCCKIVENNWFKCFIGLVTLLSTGTLAFEDIY |
| | | MDQRKTIKILLEYADMIFTYIFILEMLLKWMAYGFKAYFSNGWYRLDFVVVI |
| | | VFCLSLIGKTREELKPLISMKFLRPLRVLSQFERMKVVVRALIKTTLPTLNV |
| | | FLVCLMIWLIFSIMGVDLFAGRFYECIDPTSGERFPSSEVMNKSRCESLLEN |
| | | ESMLWENAKMNFDNVGNGFLSLLQVATENGWITIMNSAIDSVAVNIQPHFEV |
| | | NIYMYCYFINFIIFGVFLPLSMLITVIIDNFNKHKIKLGGSNIFITVKORKQ |
| | | YRRLKKLMYEDSQRPVPRPLNKLQGFIFDVVTSQAFNVIVMVLICFQAIAMM |
| | | IDTDVQSLQMSIALYWINSIFVMLYTMECILKLIAFRCFYFTIAWNIFDFMV |
| | | VIFSITGLCLPMTVGSYLVPPSLVQLILLSRIIHMLRLGKGPKVFHNLMLPL |
| | | MLSLPALLNIILLIFLVMFIYAVFGMYNFAYVKKEAGINDVSNFETFGNSML |
| | | CLFQVAIFAGWDGMLDAIFNSKWSDCDPDKINPGTQVRGDCGNPSVGIFYFV |
| | | SYILISWLIIVNMYIVVVMEFLNIASKKKNKTLSEDDFRKFFQVWKREDPDR |
| | | TQYIDSSKLSDFAAALDPPLFMAKPNKGQLIALDLPMAVGDRIHCLDILLAF |
| | | TKRVMGQDVRMEKVVSEIESGELLANPFKITCEPITTTLKRKQEAVSATIIQ |
| | | RAYKNYRLRRNDKNTSDIHMIDGDRDVHATKEGAYFDKAKEKSPIQSQI |
| 7 | hNax- | MLASPEPKGLVPFTKESFELIKQHIAKTHNEDHEEEDLKPTPDLEVGKKLPF |
| | hNav1.7_DII_ | IYGNLSQGMVSEPLEDVDPYYYKKKNTFIVLNKNRTIFRFNAASILCTLSPF |
| | chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKOLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKORVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPYWIKFKKCIYFIVMDPFVDLAITICIVLNTL |
| | | FMAMEHHPMTEEFKNVLAIGNLVFTGIFAAEMVLKLIAMDPYEYFQVGWNIF |
| | | DSLIVTLSLVELFLADVEGLSVLRSFRLLRVFKLAKSWPTLNMLIKIIGNSV |
| | | GALGNLTLVLAIIVFIFAVVGMQLFGKSYKECVCKINDDCTLPRWHMNDFFH |
| | | SFLIVFRVLCGEWIETMWDCMEVAGQAMCLIVYMMVMVIGNLVVLNLFLALL |
| | | LSSFSSDNLTAIEEDPDANNLQIAVTRIKKGINYVKOTLREFILKAFSKKPK |
| | | ISREIRQAEDLNTKKENYISNHTLAEMSKGHNFLKEKDKISGFGSSVDKHLM |
| | | EDSDGQSFIHNPSLTVTVPIAPGESDLENMNAEELSSDSDSEYSKVRLNRSS |
| | | SSECSTVDNPLPGEGEEAEAEPMNSDEPEACFTDGCVWRFSCCQVNIESGKG |
| | | KIWQNIRKTCCKIVENNWFKCFIGLVTLLSTGTLAFEDIYMDORKTIKILLE |
| | | YADMIFTYIFILEMLLKWMAYGFKAYFSNGWYRLDFVVVIVFCLSLIGKTRE |
| | | ELKPLISMKFLRPLRVLSQFERMKVVVRALIKTTLPTLNVFLVCLMIWLIES |
| | | IMGVDLFAGRFYECIDPTSGERFPSSEVMNKSRCESLLFNESMLWENAKMNF |
| | | DNVGNGFLSLLQVATFNGWITIMNSAIDSVAVNIQPHFEVNIYMYCYFINFI |
| | | IFGVFLPLSMLITVIIDNFNKHKIKLGGSNIFITVKORKQYRRLKKLMYEDS |
| | | QRPVPRPLNKLQGFIFDVVTSQAFNVIVMVLICFQAIAMMIDTDVOSLOMSI |
| | | ALYWINSIFVMLYTMECILKLIAFRCFYFTIAWNIFDFMVVIFSITGLCLPM |
| | | TVGSYLVPPSLVQLILLSRIIHMLRLGKGPKVFHNLMLPLMLSLPALLNIIL |
| | | LIFLVMFIYAVFGMYNFAYVKKEAGINDVSNFETFGNSMLCLFQVAIFAGWD |
| | | GMLDAIFNSKWSDCDPDKINPGTQVRGDCGNPSVGIFYFVSYILISWLIIVN |
| | | MYIVVVMEFLNIASKKKNKTLSEDDFRKFFQVWKRFDPDRTQYIDSSKLSDF |
| | | AAALDPPLFMAKPNKGQLIALDLPMAVGDRIHCLDILLAFTKRVMGQDVRME |
| | | KVVSEIESGFLLANPFKITCEPITTTLKRKQEAVSATIIQRAYKNYRLRRND |
| | | KNTSDIHMIDGDRDVHATKEGAYFDKAKEKSPIQSQI |
| 8 | hNax- | MLASPEPKGLVPFTKESFELIKQHIAKTHNEDHEEEDLKPTPDLEVGKKLPF |
| | hNav1.7_DIII_ | IYGNLSQGMVSEPLEDVDPYYYKKKNTFIVLNKNRTIFRFNAASILCTLSPF |
| | chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFGWALFALFRLMAQDYPEVLYHQILY |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGQDIFMTEEQKKYYNAMKKLGSKKPQKPIPRPGNKLQG |
| | | FIFDVVTSQAFNVIVMVLICFQAIAMMIDTDVQSLQMSIALYWINSIFVMLY |
| | | TMECILKLIAFRCFYFTIAWNIFDFMVVIFSITGLCLPMTVGSYLVPPSLVQ |
| | | LILLSRIIHMLRLGKGPKVFHNLMLPLMLSLPALLNIILLIFLVMFIYAVFG |
| | | MYNFAYVKKEAGINDVSNFETFGNSMLCLFQVAIFAGWDGMLDAIENSKWSD |
| | | CDPDKINPGTQVRGDCGNPSVGIFYFVSYILISWLIIVNMYIVVVMEFLNIA |
| | | SKKKNKTLSEDDFRKFFQVWKRFDPDRTQYIDSSKLSDFAAALDPPLFMAKP |
| | | NKGQLIALDLPMAVGDRIHCLDILLAFTKRVMGQDVRMEKVVSEIESGELLA |
| | | NPFKITCEPITTTLKRKQEAVSATIIQRAYKNYRLRRNDKNTSDIHMIDGDR |
| | | DVHATKEGAYFDKAKEKSPIQSQI |
| 9 | hNax- | MLASPEPKGLVPFTKESFELIKQHIAKTHNEDHEEEDLKPTPDLEVGKKLPF |
| | hNav1.7_DIV- | IYGNLSQGMVSEPLEDVDPYYYKKKNTFIVLNKNRTIFRFNAASILCTLSPF |
| | DIII- | NCIRRTTIKVLVHPFFQLFILISVLIDCVFMSLTNLPKWRPVLENTLLGIYT |
| | DIVlinker_ | FEILVKLFARGVWAGSFSFLGDPWNWLDFSVTVFEVIIRYSPLDFIPTLQTA |
| | chimera | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWE |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLENESMLWENAKMNFDNVGNGFLSLLQVATENGW |
| | | ITIMNSAIDSVAVNIQPHFEVNIYMYCYFINFIIFGVFLPLSMLITVIIDNF |
| | | NQQKKKLGGQDIFMTEEQKKYYNAMKKLGSKKPQKPIPRPGNKIQGCIFDLV |
| | | TNQAFDISIMVLICLNMVTMMVEKEGQSQHMTEVLYWINVVFIILFTGECVL |
| | | KLISLRHYYFTVGWNIFDFVVVIISIVGMFLADLIETYFVSPTLFRVIRLAR |
| | | IGRILRLVKGAKGIRTLLFALMMSLPALFNIGLLLFLVMFIYAIFGMSNFAY |
| | | VKKEDGINDMFNFETFGNSMICLFQITTSAGWDGLLAPILNSKPPDCDPKKV |
| | | HPGSSVEGDCGNPSVGIFYFVSYIIISFLVVVNMYIAVILENFSVATEESTE |
| | | PLSEDDFEMFYEVWEKFDPDATQFIEFSKLSDFAAALDPPLLIAKPNKVQLI |
| | | AMDLPMVSGDRIHCLDILFAFTKRVLGESGEMDSLRSQMEERFMSANPSKVS |
| | | YEPITTTLKRKQEDVSATVIQRAYRRYRLRQNVKNISSIYIKDGDRDDDLLN |
| | | KKDMAFDNVNENSSPEKTDATSSTTSPPSYDSVTKPDKEKYEQDRTEKEDKG |
| | | KDSKESKK |
| 10 | hNax_hNav1.7_ | MLASPEPKGLVPFTKESFELIKQHIAKTHNEDHEEEDLKPTPDLEVGKKLPF |
| | DIII_Chimera_SF_ | IYGNLSQGMVSEPLEDVDPYYYKKKNTFIVLNKNRTIFRFNAASILCTLSPF |
| | chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFSWAFLALFRLMTQDYWENLYQQTLR |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLIVFRVLCGEWIETMWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGQDIFMTEEQKKYYNAMKKLGSKKPQKPIPRPGNKLQG |
| | | FIFDVVTSQAFNVIVMVLICFQAIAMMIDTDVQSLQMSIALYWINSIFVMLY |
| | | TMECILKLIAFRCFYFTIAWNIFDFMVVIFSITGLCLPMTVGSYLVPPSLVQ |
| | | LILLSRIIHMLRLGKGPKVFHNLMLPLMLSLPALLNIILLIFLVMFIYAVFG |
| | | MYNFAYVKKEAGINDVSNFETFGNSMICLFQITTSAGWDGLLAPILNSKWSD |
| | | CDPDKINPGTQVRGDCGNPSVGIFYFVSYILISWLIIVNMYIVVVMEFLNIA |
| | | SKKKNKTLSEDDFRKFFQVWKRFDPDRTQYIDSSKLSDFAAALDPPLFMAKP |
| | | NKGQLIALDLPMAVGDRIHCLDILLAFTKRVMGQDVRMEKVVSEIESGELLA |
| | | NPFKITCEPITTTLKRKQEAVSATIIQRAYKNYRLRRNDKNTSDIHMIDGDR |
| | | DVHATKEGAYFDKAKEKSPIQSQI |
| 11 | hNax_hNav1.7_D3 | MLASPEPKGLVPFTKESFELIKQHIAKTHNEDHEEEDLKPTPDLEVGKKLPF |
| | -D4link_chimera | IYGNLSQGMVSEPLEDVDPYYYKKKNTFIVLNKNRTIFRFNAASILCTLSPF |
| | | NCIRRTTIKVLVHPFFQLFILISVLIDCVEMSLTNLPKWRPVLENTLLGIYT |
| | | FEILVKLFARGVWAGSFSFLGDPWNWLDESVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGSNIFITVKQRKQYRRLKKLMYEDSQRPVPRPLNKLQG |
| | | FIFDVVTSQAFNVIVMVLICFQAIAMMIDTDVQSLQMSIALYWINSIFVMLY |
| | | TMECILKLIAFRCFYFTIAWNIFDFMVVIFSITGLCLPMTVGSYLVPPSLVQ |
| | | LILLSRIIHMLRLGKGPKVFHNLMLPLMLSLPALLNIILLIFLVMFIYAVFG |
| | | MYNFAYVKKEAGINDVSNFETFGNSMLCLFQVAIFAGWDGMLDAIFNSKWSD |
| | | CDPDKINPGTQVRGDCGNPSVGIFYFVSYILISWLIIVNMYIVVVMEFLNIA |
| | | SKKKNKTLSEDDFRKFFQVWKRFDPDRTQYIDSSKLSDFAAALDPPLFMAKP |
| | | NKGQLIALDLPMAVGDRIHCLDILLAFTKRVMGQDVRMEKVVSEIESGELLA |
| | | NPFKITCEPITTTLKRKQEAVSATIIQRAYKNYRLRRNDKNTSDIHMIDGDR |
| | | DVHATKEGAYFDKAKEKSPIQSQI |
| 12 | hNax_hNav1.7_D3 | MLASPEPKGLVPFTKESFELIKQHIAKTHNEDHEEEDLKPTPDLEVGKKLPF |
| | -D4link- | IYGNLSQGMVSEPLEDVDPYYYKKKNTFIVLNKNRTIFRFNAASILCTLSPF |
| | PM_chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLEM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVERILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAILPTLNVFLVCLMIWLIFSIMGVDLFAGRFYEC |
| | | IDPTSGERFPSSEVMNKSRCESLLFNESMLWENAKMNFDNVGNGELSLLQVA |
| | | TFNGWITIMNSAIDSVAVNIQPHFEVNIYMYCYFINFIIFGVFLPLSMLITV |
| | | IIDNFNKHKIKLGGSNIFITVKQRKQYRRLKKLMYEDSQRPVPRPLNKLQGF |
| | | IFDVVTSQAFNVIVMVLICFQAIAMMIDTDVQSLQMSIALYWINSIFVMLYT |
| | | MECILKLIAFRCFYFTIAWNIFDFMVVIFSITGLCLPMTVGSYLVPPSLVQL |
| | | ILLSRIIHMLRLGKGPKVFHNLMLPLMLSLPALLNIILLIFLVMFIYAVFGM |
| | | YNFAYVKKEAGINDVSNFETFGNSMLCLFQVAIFAGWDGMLDAIFNSKWSDC |
| | | DPDKINPGTQVRGDCGNPSVGIFYFVSYILISWLIIVNMYIVVVMEFLNIAS |
| | | KKKNKTLSEDDFRKFFQVWKRFDPDRTQYIDSSKLSDFAAALDPPLFMAKPN |
| | | KGQLIALDLPMAVGDRIHCLDILLAFTKRVMGQDVRMEKVVSEIESGELLAN |
| | | PFKITCEPITTTLKRKQEAVSATIIQRAYKNYRLRRNDKNTSDIHMIDGDRD |
| | | VHATKEGAYFDKAKEKSPIQSQI |
| 13 | hNax_hNav1.7_D3 | MLASPEPKGLVPFTKESFELIKQHIAKTHNEDHEEEDLKPTPDLEVGKKLPF |
| | -D4link-PM- | IYGNLSQGMVSEPLEDVDPYYYKKKNTFIVLNKNRTIFRFNAASILCTLSPF |
| | S4S5link_chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFERMKVVVRALIKTTLPTLNVFLVCLMIWLIFSIMGVDLFAGRFYEC |
| | | IDPTSGERFPSSEVMNKSRCESLLFNESMLWENAKMNFDNVGNGFLSLLQVA |
| | | TFNGWITIMNSAIDSVAVNIQPHFEVNIYMYCYFINFIIFGVFLPLSMLITV |
| | | IIDNFNKHKIKLGGSNIFITVKQRKQYRRLKKLMYEDSQRPVPRPLNKLQGF |
| | | IFDVVTSQAFNVIVMVLICFQAIAMMIDTDVQSLQMSIALYWINSIFVMLYT |
| | | MECILKLIAFRCFYFTIAWNIFDFMVVIFSITGLCLPMTVGSYLVPPSLVQL |
| | | ILLSRIIHMLRLGKGPKVFHNLMLPLMLSLPALLNIILLIFLVMFIYAVFGM |
| | | YNFAYVKKEAGINDVSNFETFGNSMLCLFQVAIFAGWDGMLDAIFNSKWSDC |
| | | DPDKINPGTQVRGDCGNPSVGIFYFVSYILISWLIIVNMYIVVVMEFLNIAS |
| | | KKKNKTLSEDDFRKFFQVWKRFDPDRTQYIDSSKLSDFAAALDPPLFMAKPN |
| | | KGQLIALDLPMAVGDRIHCLDILLAFTKRVMGQDVRMEKVVSEIESGELLAN |
| | | PFKITCEPITTTLKRKQEAVSATIIQRAYKNYRLRRNDKNTSDIHMIDGDRD |
| | | VHATKEGAYFDKAKEKSPIQSQI |
| 14 | hNax_hNav1.7_V | MLASPEPKGLVPFTKESFELIKQHIAKTHNEDHEEEDLKPTPDLEVGKKLPF |
| | SD3_chimera | NCIRRTTIKVLVHPFFQLFILISVLIDCVEMSLTNLPKWRPVLENTLLGIYT |
| | | IYGNLSQGMVSEPLEDVDPYYYKKKNTFIVLNKNRTIFRFNAASILCTLSPF |
| | | FEILVKLFARGVWAGSFSFLGDPWNWLDESVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDELKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMKVVVRALIKTTLPTLNVFLVCLMIWLIFSIMGVDLFAGRFYEC |
| | | IDPTSGERFPSSEVMNKSRCESLLFNESMLWENAKMNFDNVGNGFLSLLQVA |
| | | TFNGWITIMNSAIDSVAVNIQPHFEVNIYMYCYFINFIIFGVFLPLSMLITV |
| | | IIDNFNKHKIKLGGSNIFITVKQRKQYRRLKKLMYEDSQRPVPRPLNKLQGF |
| | | IFDVVTSQAFNVIVMVLICFQAIAMMIDTDVQSLQMSIALYWINSIFVMLYT |
| | | MECILKLIAFRCFYFTIAWNIFDFMVVIFSITGLCLPMTVGSYLVPPSLVQL |
| | | ILLSRIIHMLRLGKGPKVFHNLMLPLMLSLPALLNIILLIFLVMFIYAVFGM |
| | | YNFAYVKKEAGINDVSNFETFGNSMLCLFQVAIFAGWDGMLDAIFNSKWSDC |
| | | DPDKINPGTQVRGDCGNPSVGIFYFVSYILISWLIIVNMYIVVVMEFLNIAS |
| | | KKKNKTLSEDDFRKFFQVWKRFDPDRTQYIDSSKLSDFAAALDPPLFMAKPN |
| | | KGQLIALDLPMAVGDRIHCLDILLAFTKRVMGQDVRMEKVVSEIESGELLAN |
| | | PFKITCEPITTTLKRKQEAVSATIIQRAYKNYRLRRNDKNTSDIHMIDGDRD |
| | | VHATKEGAYFDKAKEKSPIQSQI |
| 15 | hNax_hNav1.7_ | MLASPEPKGLVPFTKESFELIKQHIAKTHNEDHEEEDLKPTPDLEVGKKLPF |
| | VSD3- | IYGNLSQGMVSEPLEDVDPYYYKKKNTFIVLNKNRTIFRFNAASILCTLSPF |
| | S4S5link_chimera | NCIRRTTIKVLVHPFFQLFILISVLIDCVEMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFGWALFALFRLMAQDYPEVLYHQILY |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSTLNVFLVCLMIWLIFSIMGVDLFAGRFYEC |
| | | IDPTSGERFPSSEVMNKSRCESLLFNESMLWENAKMNFDNVGNGFLSLLQVA |
| | | TFNGWITIMNSAIDSVAVNIQPHFEVNIYMYCYFINFIIFGVFLPLSMLITV |
| | | IIDNFNKHKIKLGGSNIFITVKQRKQYRRLKKLMYEDSQRPVPRPLNKLQGF |
| | | IFDVVTSQAFNVIVMVLICFQAIAMMIDTDVQSLQMSIALYWINSIFVMLYT |
| | | MECILKLIAFRCFYFTIAWNIFDFMVVIFSITGLCLPMTVGSYLVPPSLVQL |
| | | ILLSRIIHMLRLGKGPKVFHNLMLPLMLSLPALLNIILLIFLVMFIYAVFGM |
| | | YNFAYVKKEAGINDVSNFETFGNSMLCLFQVAIFAGWDGMLDAIFNSKWSDC |
| | | DPDKINPGTQVRGDCGNPSVGIFYFVSYILISWLIIVNMYIVVVMEFLNIAS |
| | | KKKNKTLSEDDFRKFFQVWKRFDPDRTQYIDSSKLSDFAAALDPPLEMAKPN |
| | | KGQLIALDLPMAVGDRIHCLDILLAFTKRVMGQDVRMEKVVSEIESGELLAN |
| | | PFKITCEPITTTLKRKQEAVSATIIQRAYKNYRLRRNDKNTSDIHMIDGDRD |
| | | VHATKEGAYFDKAKEKSPIQSQI |
| 16 | hNax_hNav1.7_V | MLASPEPKGLVPFTKESFELIKQHIAKTHNEDHEEEDLKPTPDLEVGKKLPF |
| | SD3-S4S5link- | IYGNLSQGMVSEPLEDVDPYYYKKKNTFIVLNKNRTIFRFNAASILCTLSPF |
| | PM_chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGQDIFMTVKQRKQYRRLKKLMYEDSQRPVPRPLNKLQG |
| | | FIFDVVTSQAFNVIVMVLICFQAIAMMIDTDVQSLQMSIALYWINSIFVMLY |
| | | TMECILKLIAFRCFYFTIAWNIFDFMVVIFSITGLCLPMTVGSYLVPPSLVQ |
| | | LILLSRIIHMLRLGKGPKVFHNLMLPLMLSLPALLNIILLIFLVMFIYAVFG |
| | | MYNFAYVKKEAGINDVSNFETFGNSMLCLFQVAIFAGWDGMLDAIFNSKWSD |
| | | CDPDKINPGTQVRGDCGNPSVGIFYFVSYILISWLIIVNMYIVVVMEFLNIA |
| | | SKKKNKTLSEDDFRKFFQVWKRFDPDRTQYIDSSKLSDFAAALDPPLFMAKP |
| | | NKGQLIALDLPMAVGDRIHCLDILLAFTKRVMGQDVRMEKVVSEIESGELLA |
| | | NPFKITCEPITTTLKRKQEAVSATIIQRAYKNYRLRRNDKNTSDIHMIDGDR |
| | | DVHATKEGAYFDKAKEKSPIQSQI |
| 17 | hNax_hNav1.7_DI | |
| | II_VSD3-S3- | MLASPEPKGLVPFTKESFELIKQHIAKTHNEDHEEEDLKPTPDLEVGKKLPF |
| | S4_chimera | IYGNLSQGMVSEPLEDVDPYYYKKKNTFIVLNKNRTIFRFNAASILCTLSPF |
| | | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFTNAWCWLDFLIVDVSLVTLVANTLGYSDLGPIKSLRTLRALRPL |
| | | RALSRFERMKVVVRALIKTTLPTLNVFLVCLMIWLIFSIMGVDLFAGRFYEC |
| | | IDPTSGERFPSSEVMNKSRCESLLFNESMLWENAKMNFDNVGNGFLSLLQVA |
| | | TFNGWITIMNSAIDSVAVNIQPHFEVNIYMYCYFINFIIFGVFLPLSMLITV |
| | | IIDNFNKHKIKLGGSNIFITVKQRKQYRRLKKLMYEDSQRPVPRPLNKLQGF |
| | | IFDVVTSQAFNVIVMVLICFQAIAMMIDTDVQSLQMSIALYWINSIFVMLYT |
| | | MECILKLIAFRCFYFTIAWNIFDFMVVIFSITGLCLPMTVGSYLVPPSLVQL |
| | | ILLSRIIHMLRLGKGPKVFHNLMLPLMLSLPALLNIILLIFLVMFIYAVFGM |
| | | YNFAYVKKEAGINDVSNFETFGNSMLCLFQVAIFAGWDGMLDAIFNSKWSDC |
| | | DPDKINPGTQVRGDCGNPSVGIFYFVSYILISWLIIVNMYIVVVMEFLNIAS |
| | | KKKNKTLSEDDFRKFFQVWKRFDPDRTQYIDSSKLSDFAAALDPPLFMAKPN |
| | | KGQLIALDLPMAVGDRIHCLDILLAFTKRVMGQDVRMEKVVSEIESGELLAN |
| | | PFKITCEPITTTLKRKQEAVSATIIQRAYKNYRLRRNDKNTSDIHMIDGDRD |
| | | VHATKEGAYFDKAKEKSPIQSQI |
| 18 | hNax_hNav1.7_DI | MLASPEPKGLVPFTKESFELIKQHIAKTHNEDHEEEDLKPTPDLEVGKKLPF |
| | II_VSD3-S5- | IYGNLSQGMVSEPLEDVDPYYYKKKNTFIVLNKNRTIFRFNAASILCTLSPF |
| | S6_chimera | NCIRRTTIKVLVHPFFQLFILISVLIDCVFMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FEGMRVVVNALIGAIPSIMNVLLVCLIFWLIFSIMGVNLFAGKFYECINTTD |
| | | GSRFPASQVPNRSECFALMNVSQNVRWKNLKVNFDNVGLGYLSLLQVATFKG |
| | | WTIIMYAAVDSVNVDKQPKYEYSLYMYIYFVVFIIFGSFFTLNLFIGVIIDN |
| | | FNQQKKKLGGSNIFITVKQRKQYRRLKKLMYEDSQRPVPRPLNKLQGFIFDV |
| | | VTSQAFNVIVMVLICFQAIAMMIDTDVQSLQMSIALYWINSIFVMLYTMECI |
| | | LKLIAFRCFYFTIAWNIFDFMVVIFSITGLCLPMTVGSYLVPPSLVQLILLS |
| | | RIIHMLRLGKGPKVFHNLMLPLMLSLPALLNIILLIFLVMFIYAVFGMYNFA |
| | | YVKKEAGINDVSNFETFGNSMLCLFQVAIFAGWDGMLDAIFNSKWSDCDPDK |
| | | INPGTQVRGDCGNPSVGIFYFVSYILISWLIIVNMYIVVVMEFLNIASKKKN |
| | | KTLSEDDFRKFFQVWKRFDPDRTQYIDSSKLSDFAAALDPPLFMAKPNKGQL |
| | | IALDLPMAVGDRIHCLDILLAFTKRVMGQDVRMEKVVSEIESGFLLANPEKI |
| | | TCEPITTTLKRKQEAVSATIIQRAYKNYRLRRNDKNTSDIHMIDGDRDVHAT |
| | | KEGAYFDKAKEKSPIQSQI |
| 19 | hNax_hNav1.7_DI | MLASPEPKGLVPFTKESFELIKQHIAKTHNEDHEEEDLKPTPDLEVGKKLPF |
| | II_VSD3-S3- | IYGNLSQGMVSEPLEDVDPYYYKKKNTFIVLNKNRTIFRFNAASILCTLSPF |
| | S4loop_S4- | NCIRRTTIKVLVHPFFQLFILISVLIDCVFMSLTNLPKWRPVLENTLLGIYT |
| | S5link_S5_ | FEILVKLFARGVWAGSFSFLGDPWNWLDFSVTVFEVIIRYSPLDFIPTLQTA |
| | chimera | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLESFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFSNGWYRLDFVVVIVFCLSLIGGYSDLGLKPLISMKFLRPLRVLS |
| | | QFEGMRVVVNALIGAIPSIMNVLLVCLIFWLIFSIMGVNLFAGRFYECIDPT |
| | | SGERFPSSEVMNKSRCESLLENESMLWENAKMNFDNVGNGFLSLLQVATENG |
| | | WITIMNSAIDSVAVNIQPHFEVNIYMYCYFINFIIFGVFLPLSMLITVIIDN |
| | | FNKHKIKLGGSNIFITVKQRKQYRRLKKLMYEDSQRPVPRPLNKLQGFIFDV |
| | | VTSQAFNVIVMVLICFQAIAMMIDTDVQSLQMSIALYWINSIFVMLYTMECI |
| | | LKLIAFRCFYFTIAWNIFDFMVVIFSITGLCLPMTVGSYLVPPSLVQLILLS |
| | | RIIHMLRLGKGPKVFHNLMLPLMLSLPALLNIILLIFLVMFIYAVFGMYNFA |
| | | YVKKEAGINDVSNFETFGNSMLCLFQVAIFAGWDGMLDAIFNSKWSDCDPDK |
| | | INPGTQVRGDCGNPSVGIFYFVSYILISWLIIVNMYIVVVMEFLNIASKKKN |
| | | KTLSEDDFRKFFQVWKRFDPDRTQYIDSSKLSDFAAALDPPLFMAKPNKGQL |
| | | IALDLPMAVGDRIHCLDILLAFTKRVMGQDVRMEKVVSEIESGELLANPFKI |
| | | TCEPITTTLKRKQEAVSATIIQRAYKNYRLRRNDKNTSDIHMIDGDRDVHAT |
| | | KEGAYFDKAKEKSPIQSQI |
| 20 | hNax_ | MLASPEPKGLVPFTKESFELIKQHIAKTHNEDHEEEDLKPTPDLEVGKKLPF |
| | hNav1.7_DI | IYGNLSQGMVSEPLEDVDPYYYKKKNTFIVLNKNRTIFRFNAASILCTLSPF |
| | II_DIVS6- | NCIRRTTIKVLVHPFFQLFILISVLIDCVFMSLTNLPKWRPVLENTLLGIYT |
| | CTD_chimera | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLERMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWWNIRKTCYKIVEHSWF |
| | | ESFIVLMILLSSGALAFEDIYIERKKTIKIILEYADKIFTYIFILEMLLKWI |
| | | AYGYKTYFTNAWCWLDFLIVDVSLVTLVANTLGYSDLGPIKSLRTLRALRPL |
| | | RALSRFEGMRVVVNALIGAIPSIMNVLLVCLIFWLIFSIMGVNLFAGKFYEC |
| | | INTTDGSRFPASQVPNRSECFALMNVSQNVRWKNLKVNFDNVGLGYLSLLQV |
| | | ATFKGWTIIMYAAVDSVNVDKQPKYEYSLYMYIYFVVFIIFGSFFTLNLFIG |
| | | VIIDNFNQQKKKLGGQDIFMTEEQKKYYNAMKKLGSKKPQKPIPRPGNKLQG |
| | | FIFDVVTSQAFNVIVMVLICFQAIAMMIDTDVQSLQMSIALYWINSIFVMLY |
| | | TMECILKLIAFRCFYFTIAWNIFDFMVVIFSITGLCLPMTVGSYLVPPSLVQ |
| | | LILLSRIIHMLRLGKGPKVFHNLMLPLMLSLPALLNIILLIFLVMFIYAVFG |
| | | MYNFAYVKKEAGINDVSNFETFGNSMLCLFQVAIFAGWDGMLDAIENSKWSD |
| | | CDPDKINPGTQVRGDCGNPSVGIFYFVSYILISWLIIVNMYIAVILENFSVA |
| | | TEESTEPLSEDDFEMFYEVWEKFDPDATQFIEFSKLSDFAAALDPPLLIAKP |
| | | NKVQLIAMDLPMVSGDRIHCLDILFAFTKRVLGESGEMDSLRSQMEERFMSA |
| | | NPSKVSYEPITTTLKRKQEDVSATVIQRAYRRYRLRQNVKNISSIYIKDGDR |
| | | DDDLLNKKDMAFDNVNENSSPEKTDATSSTTSPPSYDSVTKPDKEKYEQDRT |
| | | EKEDKGKDSKESKK |
| 21 | Nax_Nav1.7_D1- | MLASPEPKGLVPFTKESFELIKQHIAKTHNEDHEEEDLKPTPDLEVGKKLPF |
| | D2_linker_chimera | IYGNLSQGMVSEPLEDVDPYYYKKKNTFIVLNKNRTIFRFNAASILCTLSPF |
| | | NCIRRTTIKVLVHPFFQLFILISVLIDCVFMSLTNLPKWRPVLENTLLGIYT |
| | | FEILVKLFARGVWAGSFSFLGDPWNWLDESVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGQKELEFQQMLDRLKKEQEEAEAIAA |
| | | AAAEYTSIRRSRIMGLSESSSETSKLSSKSAKERRNRRKKKNQKKLSSGEEK |
| | | GDAEKLSKSESEDSIRRKSFHLGVEGHRRAHEKRLSTPNQSPLSIRGSLFSA |
| | | RRSSRTSLFSFKGRGRDIGSETEFADDEHSIFGDNESRRGSLFVPHRPQERR |
| | | SSNISQASRSPPMLPVNGKMHSAVDCNGVVSLVDGRSALMLPNGQLLPEVII |
| | | DKATSDDSGTTNQIHKKRRCSSYLLSEDMLNDPNLRQRAMSRASILTNTVEE |
| | | LEESRQKCPPWWYRFAHKFLIWNFTDLFLIICIILNVCFLTLEHYPMSKQTN |
| | | TLLNIGNLVFIGIFTAEMIFKIIAMHPYGYFQVGWNIFDSMIVFHGLIELCL |
| | | ANVAGMALLRLFRMLRIFKLGKYWPTFQILMWSLSNSWVALKDLVLLLFTFI |
| | | FFSAAFGMKLFGKNYEEFVCHIDKDCQLPRWHMHDFFHSFLNVFRILCGEWV |
| | | ETLWDCMEVAGQSWCIPFYLMVILIGNLLVLYLFLALVSSFSSCKDVTAEEN |
| | | NEAKNLQLAVARIKKGINYVLLKILCKTQNVPKDTMDHVNEVYVKEDISDHT |
| | | LSELSNTQDFLKDKEKSSGTEKNATENESQSLIPSPSVSETVPIASGESDIE |
| | | NLDNKEIQSKSGDGGSKEKIKQSSSSECSTVDIAISEEEEMFYGGERSKHLK |
| | | NGCRRGSSLGQISGASKKGKIWQNIRKTCCKIVENNWFKCFIGLVTLLSTGT |
| | | LAFEDIYMDQRKTIKILLEYADMIFTYIFILEMLLKWMAYGFKAYFSNGWYR |
| | | LDFVVVIVFCLSLIGKTREELKPLISMKFLRPLRVLSQFERMKVVVRALIKT |
| | | TLPTLNVFLVCLMIWLIFSIMGVDLFAGRFYECIDPTSGERFPSSEVMNKSR |
| | | CESLLENESMLWENAKMNFDNVGNGELSLLQVATENGWITIMNSAIDSVAVN |
| | | IQPHFEVNIYMYCYFINFIIFGVFLPLSMLITVIIDNENKHKIKLGGSNIFI |
| | | TVKQRKQYRRLKKLMYEDSQRPVPRPLNKLQGFIFDVVTSQAFNVIVMVLIC |
| | | FQAIAMMIDTDVQSLQMSIALYWINSIFVMLYTMECILKLIAFRCFYFTIAW |
| | | NIFDFMVVIFSITGLCLPMTVGSYLVPPSLVQLILLSRIIHMLRLGKGPKVE |
| | | HNLMLPLMLSLPALLNIILLIFLVMFIYAVFGMYNFAYVKKEAGINDVSNFE |
| | | TFGNSMLCLFQVAIFAGWDGMLDAIFNSKWSDCDPDKINPGTQVRGDCGNPS |
| | | VGIFYFVSYILISWLIIVNMYIVVVMEFLNIASKKKNKTLSEDDFRKFFQVW |
| | | KRFDPDRTQYIDSSKLSDFAAALDPPLEMAKPNKGQLIALDLPMAVGDRIHC |
| | | LDILLAFTKRVMGQDVRMEKVVSEIESGELLANPFKITCEPITTTLKRKQEA |
| | | VSATIIQRAYKNYRLRRNDKNTSDIHMIDGDRDVHATKEGAYFDKAKEKSPI |
| | | QSQI |
| 22 | Nax_Nav1.7_D2- | MLASPEPKGLVPFTKESFELIKQHIAKTHNEDHEEEDLKPTPDLEVGKKLPF |
| | D3_linker_chimera | NCIRRTTIKVLVHPFFQLFILISVLIDCVFMSLTNLPKWRPVLENTLLGIYT |
| | | IYGNLSQGMVSEPLEDVDPYYYKKKNTFIVLNKNRTIFRENAASILCTLSPF |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALS |
| | | FSSDNLTAIEEDPDANNLQIAVTRIKKGINYVKQTLREFILKAFSKKPKISR |
| | | EIRQAEDLNTKKENYISNHTLAEMSKGHNFLKEKDKISGFGSSVDKHLMEDS |
| | | DGQSFIHNPSLTVTVPIAPGESDLENMNAEELSSDSDSEYSKVRLNRSSSSE |
| | | CSTVDNPLPGEGEEAEAEPMNSDEPEACFTDGCVWRFSCCQVNIESGKGKKG |
| | | KIWQNIRKTCCKIVENNWFKCFIGLVTLLSTGTLAFEDIYMDQRKTIKILLE |
| | | YADMIFTYIFILEMLLKWMAYGFKAYFSNGWYRLDFVVVIVFCLSLIGKTRE |
| | | ELKPLISMKFLRPLRVLSQFERMKVVVRALIKTTLPTLNVFLVCLMIWLIFS |
| | | IMGVDLFAGRFYECIDPTSGERFPSSEVMNKSRCESLLFNESMLWENAKMNF |
| | | DNVGNGFLSLLQVATFNGWITIMNSAIDSVAVNIQPHFEVNIYMYCYFINFI |
| | | IFGVFLPLSMLITVIIDNFNKHKIKLGGSNIFITVKQRKQYRRLKKLMYEDS |
| | | QRPVPRPLNKLQGFIFDVVTSQAFNVIVMVLICFQAIAMMIDTDVQSLQMSI |
| | | ALYWINSIFVMLYTMECILKLIAFRCFYFTIAWNIFDFMVVIFSITGLCLPM |
| | | TVGSYLVPPSLVQLILLSRIIHMLRLGKGPKVFHNLMLPLMLSLPALLNIIL |
| | | LIFLVMFIYAVFGMYNFAYVKKEAGINDVSNFETFGNSMLCLFQVAIFAGWD |
| | | GMLDAIFNSKWSDCDPDKINPGTQVRGDCGNPSVGIFYFVSYILISWLIIVN |
| | | MYIVVVMEFLNIASKKKNKTLSEDDFRKFFQVWKRFDPDRTQYIDSSKLSDF |
| | | AAALDPPLFMAKPNKGQLIALDLPMAVGDRIHCLDILLAFTKRVMGQDVRME |
| | | KVVSEIESGFLLANPFKITCEPITTTLKRKQEAVSATIIQRAYKNYRLRRND |
| | | KNTSDIHMIDGDRDVHATKEGAYFDKAKEKSPIQSQI |
| 23 | Nax_Nav1.7_D3- | MLASPEPKGLVPFTKESFELIKQHIAKTHNEDHEEEDLKPTPDLEVGKKLPF |
| | D4_linker_chimera | IYGNLSQGMVSEPLEDVDPYYYKKKNTFIVLNKNRTIFRFNAASILCTLSPF |
| | | NCIRRTTIKVLVHPFFQLFILISVLIDCVEMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIE |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWE |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLFNESMLWENAKMNFDNVGNGELSLLQVATENGW |
| | | ITIMNSAIDSVAVNIQPHFEVNIYMYCYFINFIIFGVFLPLSMLILGGQDIF |
| | | MTEEQKKYYNAMKKLGSKKPQKPIPRPGNKLQGFIFDVVTSQAFNVIVMVLI |
| | | CFQAIAMMIDTDVQSLQMSIALYWINSIFVMLYTMECILKLIAFRCFYFTIA |
| | | WNIFDFMVVIFSITGLCLPMTVGSYLVPPSLVQLILLSRIIHMLRLGKGPKV |
| | | FHNLMLPLMLSLPALLNIILLIFLVMFIYAVFGMYNFAYVKKEAGINDVSNF |
| | | ETFGNSMLCLFQVAIFAGWDGMLDAIFNSKWSDCDPDKINPGTQVRGDCGNP |
| | | SVGIFYFVSYILISWLIIVNMYIVVVMEFLNIASKKKNKTLSEDDFRKFFQV |
| | | WKRFDPDRTQYIDSSKLSDFAAALDPPLFMAKPNKGQLIALDLPMAVGDRIH |
| | | CLDILLAFTKRVMGQDVRMEKVVSEIESGELLANPFKITCEPITTTLKRKQE |
| | | AVSATIIQRAYKNYRLRRNDKNTSDIHMIDGDRDVHATKEGAYEDKAKEKSP |
| | | IQSQI |
| 24 | Nax_Nav1.7_ | MAMLPPPGPQSFVHFTKQSLALIEQRIAERKSKEPKEEKKDDDEEAPKPSSD |
| | ultimateloop_ | LEAGKQLPFIYGDIPPGMVSEPLEDLDPYYADKKTFIVLNKGKTIFRENATP |
| | chimera | ALYMLSPFSPFFQLFILISVLIDCVFMSLTNLPKWRPVLENTLLGIYTFEIL |
| | | VKLFARGVWAGSFSFLGDPWNWLDFSVTVFEVIIRYSPLDFIPTLQTARTLR |
| | | ILKIIPLNQGLKSLVGVLIHCLKQLIGVIILTLFFLSIFSLIGMGLFMGNLK |
| | | HKCFRWPQENENETLHNRTGNPYYIRETENFYYLEGERYALLCGNRTDAGQC |
| | | PEGYVCVKAGINPDQGFTNFDSFGWALFALFRLMAQDYPEVLYHQILYASGK |
| | | VYMIFFVVVSFLFSFYMASLFLGQKELEFQQMLDRLKKEQEEAEAIAAAAAE |
| | | YTSIRRSRIMGLSESSSETSKLSSKSAKERRNRRKKKNQKKLSSGEEKGDAE |
| | | KLSKSESEDSIRRKSFHLGVEGHRRAHEKRLSTPNQSPLSIRGSLESARRSS |
| | | RTSLFSFKGRGRDIGSETEFADDEHSIFGDNESRRGSLFVPHRPQERRSSNI |
| | | SQASRSPPMLPVNGKMHSAVDCNGVVSLVDGRSALMLPNGQLLPEVIIDKAT |
| | | SDDSGTTNQIHKKRRCSSYLLSEDMLNDPNLRQRAMSRASILTNTVEELEES |
| | | RQKCPPWWYRFAHKFLIWNFTDLFLIICIILNVCFLTLEHYPMSKQTNTLLN |
| | | IGNLVFIGIFTAEMIFKIIAMHPYGYFQVGWNIFDSMIVFHGLIELCLANVA |
| | | GMALLRLFRMLRIFKLGKYWPTFQILMWSLSNSWVALKDLVLLLFTFIFFSA |
| | | AFGMKLFGKNYEEFVCHIDKDCQLPRWHMHDFFHSFLNVFRILCGEWVETLW |
| | | DCMEVAGQSWCIPFYLMVILIGNLLVLYLFLALSFSSDNLTAIEEDPDANNL |
| | | QIAVTRIKKGINYVKQTLREFILKAFSKKPKISREIRQAEDLNTKKENYISN |
| | | HTLAEMSKGHNFLKEKDKISGFGSSVDKHLMEDSDGQSFIHNPSLTVTVPIA |
| | | PGESDLENMNAEELSSDSDSEYSKVRLNRSSSSECSTVDNPLPGEGEEAEAE |
| | | PMNSDEPEACFTDGCVWRFSCCQVNIESGKGKKGKIWQNIRKTCCKIVENNW |
| | | FKCFIGLVTLLSTGTLAFEDIYMDQRKTIKILLEYADMIFTYIFILEMLLKW |
| | | MAYGFKAYFSNGWYRLDFVVVIVFCLSLIGKTREELKPLISMKFLRPLRVLS |
| | | QFERMKVVVRALIKTTLPTLNVFLVCLMIWLIFSIMGVDLFAGRFYECIDPT |
| | | SGERFPSSEVMNKSRCESLLFNESMLWENAKMNFDNVGNGFLSLLQVATENG |
| | | WITIMNSAIDSVAVNIQPHFEVNIYMYCYFINFIIFGVFLPLSMLILGGQDI |
| | | FMTEEQKKYYNAMKKLGSKKPQKPIPRPGNKLQGFIFDVVTSQAFNVIVMVL |
| | | ICFQAIAMMIDTDVQSLQMSIALYWINSIFVMLYTMECILKLIAFRCFYFTI |
| | | AWNIFDFMVVIFSITGLCLPMTVGSYLVPPSLVQLILLSRIIHMLRLGKGPK |
| | | VFHNLMLPLMLSLPALLNIILLIFLVMFIYAVFGMYNFAYVKKEAGINDVSN |
| | | FETFGNSMLCLFQVAIFAGWDGMLDAIFNSKWSDCDPDKINPGTQVRGDCGN |
| | | PSVGIFYFVSYILISWLIIVNMYIVVVESTEPLSEDDFEMFYEVWEKFDPDA |
| | | TQFIEFSKLSDFAAALDPPLLIAKPNKVQLIAMDLPMVSGDRIHCLDILFAF |
| | | TKRVLGESGEMDSLRSQMEERFMSANPSKVSYEPITTTLKRKQEDVSATVIQ |
| | | RAYRRYRLRQNVKNISSIYIKDGDRDDDLLNKKDMAFDNVNENSSPEKTDAT |
| | | SSTTSPPSYDSVTKPDKEKYEQDRTEKEDKGKDSKESKK |
| 25 | Nax_QTT | MLASPEPKGLVPFTKESFELIKQHIAKTHNEDHEEEDLKPTPDLEVGKKLPF |
| | | IYGNLSQGMVSEPLEDVDPYYYKKKNTFIVLNKNRTIFRFNAASILCTLSPF |
| | | NCIRRTTIKVLVHPFFQLFILISVLIDCVEMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLQLALV |
| | | SSFSSCKDVTAEENNEAKNLQLAVARIKKGINYVLLKILCKTQNVPKDTMDH |
| | | VNEVYVKEDISDHTLSELSNTQDELKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLENESMLWENAKMNFDNVGNGFLSLLQVATENGW |
| | | ITIMNSAIDSVAVNIQPHFEVNIYMYCYFINFIIFGVFLPLSMLTTVIIDNF |
| | | NKHKIKLGGSNIFITVKQRKQYRRLKKLMYEDSQRPVPRPLNKLQGFIFDVV |
| | | TSQAFNVIVMVLICFQAIAMMIDTDVQSLQMSIALYWINSIFVMLYTMECIL |
| | | KLIAFRCFYFTIAWNIFDFMVVIFSITGLCLPMTVGSYLVPPSLVQLILLSR |
| | | IIHMLRLGKGPKVFHNLMLPLMLSLPALLNIILLIFLVMFIYAVFGMYNFAY |
| | | VKKEAGINDVSNFETFGNSMLCLFQVAIFAGWDGMLDAIFNSKWSDCDPDKI |
| | | NPGTQVRGDCGNPSVGIFYFVSYILISWLIIVNMYTVVVMEFLNIASKKKNK |
| | | TLSEDDFRKFFQVWKRFDPDRTQYIDSSKLSDFAAALDPPLFMAKPNKGQLI |
| | | ALDLPMAVGDRIHCLDILLAFTKRVMGQDVRMEKVVSEIESGELLANPFKIT |
| | | CEPITTTLKRKQEAVSATIIQRAYKNYRLRRNDKNTSDIHMIDGDRDVHATK |
| | | EGAYFDKAKEKSPIQSQI |
| 26 | Nax_EEE | MLASPEPKGLVPFTKESFELIKQHIAKTHNEDHEEEDLKPTPDLEVGKKLPF |
| | | IYGNLSQGMVSEPLEDVDPYYYKKKNTFIVLNKNRTIFRFNAASILCTLSPF |
| | | NCIRRTTIKVLVHPFFQLFILISVLIDCVEMSLTNLPKWRPVLENTLLGIYT |
| | | FEILVKLFARGVWAGSFSFLGDPWNWLDFSVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNEDSFGWALFALFRLMAQDYPEVLYHQILY |
| | | ASGKVYMIFFVVVSFLFSFYMASLFEGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWE |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLFNESMLWENAKMNFDNVGNGELSLLQVATENGW |
| | | ITIMNSAIDSVAVNIQPHFEVNIYMYCYFINFIIFGVFLPLSMLETVIIDNE |
| | | NKHKIKLGGSNIFITVKQRKQYRRLKKLMYEDSQRPVPRPLNKLQGFIFDVV |
| | | TSQAFNVIVMVLICFQAIAMMIDTDVQSLQMSIALYWINSIFVMLYTMECIL |
| | | KLIAFRCFYFTIAWNIFDFMVVIFSITGLCLPMTVGSYLVPPSLVQLILLSR |
| | | IIHMLRLGKGPKVFHNLMLPLMLSLPALLNIILLIFLVMFIYAVFGMYNFAY |
| | | VKKEAGINDVSNFETFGNSMLCLFQVAIFAGWDGMLDAIFNSKWSDCDPDKI |
| | | NPGTQVRGDCGNPSVGIFYFVSYILISWLIIVNMYEVVVMEFLNIASKKKNK |
| | | TLSEDDFRKFFQVWKRFDPDRTQYIDSSKLSDFAAALDPPLFMAKPNKGQLI |
| | | ALDLPMAVGDRIHCLDILLAFTKRVMGQDVRMEKVVSEIESGELLANPFKIT |
| | | CEPITTTLKRKQEAVSATIIQRAYKNYRLRRNDKNTSDIHMIDGDRDVHATK |
| | | EGAYFDKAKEKSPIQSQI |
| 27 | Human Nax | MLASPEPKGLVPFTKESFELIKQHIAKTHNEDHEEEDLKPTPDLEVGKKLPF |
| | (FIG. 9) | IYGNLSQGMVSEPLEDVDPYYYKKKNTFIVLNKNRTIFRFNAASILCTLSPF |
| | | NCIRRTTIKVLVHPFFQLFILISVLIDCVFMSLTNLPKWRPVLENTLLGIYT |
| | | FEILVKLFARGVWAGSFSFLGDPWNWLDESVTVFEVIIRYSPLDFIPTLQTA |
| | | RTLRILKIIPLNQGLKSLVGVLIHCLKQLIGVIILTLFFLSIFSLIGMGLFM |
| | | GNLKHKCFRWPQENENETLHNRTGNPYYIRETENFYYLEGERYALLCGNRTD |
| | | AGQCPEGYVCVKAGINPDQGFTNFDSFGWALFALFRLMAQDYPEVLYHQILY |
| | | ASGKVYMIFFVVVSFLFSFYMASLFLGILAMAYEEEKQRVGEISKKIEPKFQ |
| | | QTGKELQEGNETDEAKTIQIEMKKRSPISTDTSLDVLEDATLRHKEELEKSK |
| | | KICPLYWYKFAKTFLIWNCSPCWLKLKEFVHRIIMAPFTDLFLIICIILNVC |
| | | FLTLEHYPMSKQTNTLLNIGNLVFIGIFTAEMIFKIIAMHPYGYFQVGWNIF |
| | | DSMIVFHGLIELCLANVAGMALLRLFRMLRIFKLGKYWPTFQILMWSLSNSW |
| | | VALKDLVLLLFTFIFFSAAFGMKLFGKNYEEFVCHIDKDCQLPRWHMHDFFH |
| | | SFLNVFRILCGEWVETLWDCMEVAGQSWCIPFYLMVILIGNLLVLYLFLALV |
| | | SSFSSCKDVTAEENNEAKNLQLAVARIKKGINYVLLKILCKTQNVPKDTMDH |
| | | VNEVYVKEDISDHTLSELSNTQDFLKDKEKSSGTEKNATENESQSLIPSPSV |
| | | SETVPIASGESDIENLDNKEIQSKSGDGGSKEKIKQSSSSECSTVDIAISEE |
| | | EEMFYGGERSKHLKNGCRRGSSLGQISGASKKGKIWQNIRKTCCKIVENNWF |
| | | KCFIGLVTLLSTGTLAFEDIYMDQRKTIKILLEYADMIFTYIFILEMLLKWM |
| | | AYGFKAYFSNGWYRLDFVVVIVFCLSLIGKTREELKPLISMKFLRPLRVLSQ |
| | | FERMKVVVRALIKTTLPTLNVFLVCLMIWLIFSIMGVDLFAGRFYECIDPTS |
| | | GERFPSSEVMNKSRCESLLFNESMLWENAKMNFDNVGNGFLSLLQVATENGW |
| | | ITIMNSAIDSVAVNIQPHFEVNIYMYCYFINFIIFGVFLPLSMLITVIIDNF |
| | | NKHKIKLGGSNIFITVKQRKQYRRLKKLMYEDSQRPVPRPLNKLQGFIFDVV |
| | | TSQAFNVIVMVLICFQAIAMMIDTDVQSLQMSIALYWINSIFVMLYTMECIL |
| | | KLIAFRCFYFTIAWNIFDFMVVIFSITGLCLPMTVGSYLVPPSLVQLILLSR |
| | | IIHMLRLGKGPKVFHNLMLPLMLSLPALLNIILLIFLVMFIYAVFGMYNFAY |
| | | VKKEAGINDVSNFETFGNSMLCLFQVAIFAGWDGMLDAIFNSKWSDCDPDKI |
| | | NPGTQVRGDCGNPSVGIFYFVSYILISWLIIVNMYIVVVMEFLNIASKKKNK |
| | | TLSEDDFRKFFQVWKRFDPDRTQYIDSSKLSDFAAALDPPLFMAKPNKGQLI |
| | | ALDLPMAVGDRIHCLDILLAFTKRVMGQDVRMEKVVSEIESGELLANPFKIT |
| | | CEPITTTLKRKQEAVSATIIQRAYKNYRLRRNDKNTSDIHMIDGDRDVHATK |
| | | EGAYFDKAKEKSPIQSQI |
| 28 | Human Nav 1.1 | MEQTVLVPPGPDSFNFFTRESLAAIERRIAEEKAKNPKPDKKDDDENGPKPN |
| | (FIG. 9) | SDLEAGKNLPFIYGDIPPEMVSEPLEDLDPYYINKKTFIVLNKGKAIFRESA |
| | | TSALYILTPFNPLRKIAIKILVHSLFSMLIMCTILTNCVFMTMSNPPDWTKN |
| | | VEYTFTGIYTFESLIKIIARGFCLEDFTFLRDPWNWLDFTVITFAYVTEFVD |
| | | LGNVSALRTFRVLRALKTISVIPGLKTIVGALIQSVKKLSDVMILTVFCLSV |
| | | FALIGLQLFMGNLRNKCIQWPPTNASLEEHSIEKNITVNYNGTLINETVFEF |
| | | DWKSYIQDSRYHYFLEGFLDALLCGNSSDAGQCPEGYMCVKAGRNPNYGYTS |
| | | FDTFSWAFLSLFRLMTQDFWENLYQLTLRAAGKTYMIFFVLVIFLGSFYLIN |
| | | LILAVVAMAYEEQNQATLEEAEQKEAEFQQMIEQLKKQQEAAQQAATATASE |
| | | HSREPSAAGRLSDSSSEASKLSSKSAKERRNRRKKRKQKEQSGGEEKDEDEF |
| | | QKSESEDSIRRKGFRFSIEGNRLTYEKRYSSPHQSLLSIRGSLFSPRRNSRT |
| | | SLFSFRGRAKDVGSENDFADDEHSTFEDNESRRDSLFVPRRHGERRNSNLSQ |
| | | TSRSSRMLAVFPANGKMHSTVDCNGVVSLVGGPSVPTSPVGQLLPEVIIDKP |
| | | ATDDNGTTTETEMRKRRSSSFHVSMDFLEDPSQRQRAMSIASILTNTVEELE |
| | | ESRQKCPPCWYKFSNIFLIWDCSPYWLKVKHVVNLVVMDPFVDLAITICIVL |
| | | NTLFMAMEHYPMTDHFNNVLTVGNLVFTGIFTAEMFLKIIAMDPYYYFQEGW |
| | | NIFDGFIVTLSLVELGLANVEGLSVLRSFRLLRVFKLAKSWPTLNMLIKIIG |
| | | NSVGALGNLTLVLAIIVFIFAVVGMQLFGKSYKDCVCKIASDCQLPRWHMND |
| | | FFHSFLIVFRVLCGEWIETMWDCMEVAGQAMCLTVFMMVMVIGNLVVLNLFL |
| | | ALLLSSFSADNLAATDDDNEMNNLQIAVDRMHKGVAYVKRKIYEFIQQSFIR |
| | | KQKILDEIKPLDDLNNKKDSCMSNHTAEIGKDLDYLKDVNGTTSGIGTGSSV |
| | | EKYIIDESDYMSFINNPSLTVTVPIAVGESDFENLNTEDESSESDLEESKEK |
| | | LNESSSSSEGSTVDIGAPVEEQPVVEPEETLEPEACFTEGCVQRFKCCQINV |
| | | EEGRGKQWWNLRRTCFRIVEHNWFETFIVEMILLSSGALAFEDIYIDQRKTI |
| | | KTMLEYADKVFTYIFILEMLLKWVAYGYQTYFTNAWCWLDFLIVDVSLVSLT |
| | | ANALGYSELGAIKSLRTLRALRPLRALSRFEGMRVVVNALLGAIPSIMNVLL |
| | | VCLIFWLIFSIMGVNLFAGKFYHCINTTTGDREDIEDVNNHTDCLKLIERNE |
| | | TARWKNVKVNFDNVGFGYLSLLQVATFKGWMDIMYAAVDSRNVELQPKYEES |
| | | LYMYLYFVIFIIFGSFFTLNLFIGVIIDNFNQQKKKFGGQDIFMTEEQKKYY |
| | | NAMKKLGSKKPQKPIPRPGNKFQGMVFDFVTRQVFDISIMILICLNMVTMMV |
| | | ETDDQSEYVTTILSRINLVFIVLFTGECVLKLISLRHYYFTIGWNIFDFVVV |
| | | ILSIVGMFLAELIEKYFVSPTLFRVIRLARIGRILRLIKGAKGIRTLLFALM |
| | | MSLPALFNIGLLLFLVMFIYAIFGMSNFAYVKREVGIDDMENFETEGNSMIC |
| | | LFQITTSAGWDGLLAPILNSKPPDCDPNKVNPGSSVKGDCGNPSVGIFFFVS |
| | | YIIISFLVVVNMYIAVILENFSVATEESAEPLSEDDFEMFYEVWEKFDPDAT |
| | | QFMEFEKLSQFAAALEPPLNLPQPNKLQLIAMDLPMVSGDRIHCLDILFAFT |
| | | KRVLGESGEMDALRIQMEERFMASNPSKVSYQPITTTLKRKQEEVSAVIIQR |
| | | AYRRHLLKRTVKQASFTYNKNKIKGGANLLIKEDMIIDRINENSITEKTDLT |
| | | MSTAACPPSYDRVTKPIVEKHEQEGKDEKAKGK |
| 29 | Human Nav 1.2 | MAQSVLVPPGPDSFRFFTRESLAAIEQRIAEEKAKRPKQERKDEDDENGPKP |
| | (FIG. 9) | NSDLEAGKSLPFIYGDIPPEMVSVPLEDLDPYYINKKTFIVLNKGKAISRFS |
| | | ATPALYILTPFNPIRKLAIKILVHSLFNMLIMCTILTNCVFMTMSNPPDWTK |
| | | NVEYTFTGIYTFESLIKILARGFCLEDFTFLRDPWNWLDFTVITFAYVTEFV |
| | | DLGNVSALRTFRVLRALKTISVIPGLKTIVGALIQSVKKLSDVMILTVFCLS |
| | | VFALIGLQLFMGNLRNKCLQWPPDNSSFEINITSFFNNSLDGNGTTENRTVS |
| | | IFNWDEYIEDKSHFYFLEGQNDALLCGNSSDAGQCPEGYICVKAGRNPNYGY |
| | | TSFDTFSWAFLSLFRLMTQDFWENLYQLTLRAAGKTYMIFFVLVIFLGSFYL |
| | | INLILAVVAMAYEEQNQATLEEAEQKEAEFQQMLEQLKKQQEEAQAAAAAAS |
| | | AESRDFSGAGGIGVFSESSSVASKLSSKSEKELKNRRKKKKQKEQSGEEEKN |
| | | DRVRKSESEDSIRRKGFRFSLEGSRLTYEKRFSSPHQSLLSIRGSLFSPRRN |
| | | SRASLFSFRGRAKDIGSENDFADDEHSTFEDNDSRRDSLFVPHRHGERRHSN |
| | | VSQASRASRVLPILPMNGKMHSAVDCNGVVSLVGGPSTLTSAGQLLPEGTTT |
| | | ETEIRKRRSSSYHVSMDLLEDPTSRQRAMSIASILTNTMEELEESRQKCPPC |
| | | WYKFANMCLIWDCCKPWLKVKHLVNLVVMDPFVDLAITICIVLNTLFMAMEH |
| | | YPMTEQFSSVLSVGNLVFTGIFTAEMFLKIIAMDPYYYFQEGWNIFDGFIVS |
| | | LSLMELGLANVEGLSVLRSFRLLRVFKLAKSWPTLNMLIKIIGNSVGALGNL |
| | | TLVLAIIVFIFAVVGMQLFGKSYKECVCKISNDCELPRWHMHDFFHSFLIVF |
| | | RVLCGEWIETMWDCMEVAGQTMCLTVFMMVMVIGNLVVLNLFLALLLSSFSS |
| | | DNLAATDDDNEMNNLQIAVGRMQKGIDFVKRKIREFIQKAFVRKQKALDEIK |
| | | PLEDLNNKKDSCISNHTTIEIGKDLNYLKDGNGTTSGIGSSVEKYVVDESDY |
| | | MSFINNPSLTVTVPIAVGESDFENLNTEEFSSESDMEESKEKLNATSSSEGS |
| | | TVDIGAPAEGEQPEVEPEESLEPEACFTEDCVRKFKCCQISIEEGKGKLWWN |
| | | LRKTCYKIVEHNWFETFIVEMILLSSGALAFEDIYIEQRKTIKTMLEYADKV |
| | | FTYIFILEMLLKWVAYGFQVYFTNAWCWLDFLIVDVSLVSLTANALGYSELG |
| | | AIKSLRTLRALRPLRALSRFEGMRVVVNALLGAIPSIMNVLLVCLIFWLIES |
| | | IMGVNLFAGKFYHCINYTTGEMFDVSVVNNYSECKALIESNQTARWKNVKVN |
| | | FDNVGLGYLSLLQVATFKGWMDIMYAAVDSRNVELQPKYEDNLYMYLYFVIF |
| | | IIFGSFFTLNLFIGVIIDNFNQQKKKFGGQDIFMTEEQKKYYNAMKKLGSKK |
| | | PQKPIPRPANKFQGMVFDFVTKQVFDISIMILICLNMVTMMVETDDQSQEMT |
| | | NILYWINLVFIVLFTGECVLKLISLRYYYFTIGWNIFDFVVVILSIVGMFLA |
| | | ELIEKYFVSPTLFRVIRLARIGRILRLIKGAKGIRTLLFALMMSLPALFNIG |
| | | LLLFLVMFIYAIFGMSNFAYVKREVGIDDMENFETFGNSMICLFQITTSAGW |
| | | DGLLAPILNSGPPDCDPDKDHPGSSVKGDCGNPSVGIFFFVSYIIISFLVVV |
| | | NMYIAVILENFSVATEESAEPLSEDDFEMFYEVWEKFDPDATQFIEFAKLSD |
| | | FADALDPPLLIAKPNKVQLIAMDLPMVSGDRIHCLDILFAFTKRVLGESGEM |
| | | DALRIQMEERFMASNPSKVSYEPITTTLKRKQEEVSAIIIQRAYRRYLLKQK |
| | | VKKVSSIYKKDKGKECDGTPIKEDTLIDKLNENSTPEKTDMTPSTTSPPSYD |
| | | SVTKPEKEKFEKDKSEKEDKGKDIRESKK |
| 30 | Human Nav 1.3 | MAQALLVPPGPESFRLFTRESLAAIEKRAAEEKAKKPKKEQDNDDENKPKPN |
| | (FIG. 9) | SDLEAGKNLPFIYGDIPPEMVSEPLEDLDPYYINKKTFIVMNKGKAIFRESA |
| | | TSALYILTPLNPVRKIAIKILVHSLFSMLIMCTILTNCVFMTLSNPPDWTKN |
| | | VEYTFTGIYTFESLIKILARGFCLEDFTFLRDPWNWLDESVIVMAYVTEFVS |
| | | LGNVSALRTFRVLRALKTISVIPGLKTIVGALIQSVKKLSDVMILTVFCLSV |
| | | FALIGLQLFMGNLRNKCLQWPPSDSAFETNTTSYFNGTMDSNGTFVNVTMST |
| | | FNWKDYIGDDSHFYVLDGQKDPLLCGNGSDAGQCPEGYICVKAGRNPNYGYT |
| | | SFDTFSWAFLSLFRLMTQDYWENLYQLTLRAAGKTYMIFFVLVIFLGSFYLV |
| | | NLILAVVAMAYEEQNQATLEEAEQKEAEFQQMLEQLKKQQEEAQAVAAASAA |
| | | SRDESGIGGLGELLESSSEASKLSSKSAKEWRNRRKKRRQREHLEGNNKGER |
| | | DSFPKSESEDSVKRSSFLFSMDGNRLTSDKKFCSPHQSLLSIRGSLESPRRN |
| | | SKTSIFSFRGRAKDVGSENDFADDEHSTFEDSESRRDSLFVPHRHGERRNSN |
| | | VSQASMSSRMVPGLPANGKMHSTVDCNGVVSLVGGPSALTSPTGQLPPEGTT |
| | | TETEVRKRRLSSYQISMEMLEDSSGRQRAVSIASILTNTMEELEESRQKCPP |
| | | CWYRFANVFLIWDCCDAWLKVKHLVNLIVMDPFVDLAITICIVLNTLFMAME |
| | | HYPMTEQFSSVLTVGNLVFTGIFTAEMVLKIIAMDPYYYFQEGWNIFDGIIV |
| | | SLSLMELGLSNVEGLSVLRSFRLLRVFKLAKSWPTLNMLIKIIGNSVGALGN |
| | | LTLVLAIIVFIFAVVGMQLFGKSYKECVCKINDDCTLPRWHMNDFFHSFLIV |
| | | FRVLCGEWIETMWDCMEVAGQTMCLIVFMLVMVIGNLVVLNLFLALLLSSES |
| | | SDNLAATDDDNEMNNLQIAVGRMQKGIDYVKNKMRECFQKAFFRKPKVIEIH |
| | | EGNKIDSCMSNNTGIEISKELNYLRDGNGTTSGVGTGSSVEKYVIDENDYMS |
| | | FINNPSLTVTVPIAVGESDFENLNTEEFSSESELEESKEKLNATSSSEGSTV |
| | | DVVLPREGEQAETEPEEDLKPEACFTEGCIKKFPFCQVSTEEGKGKIWWNLR |
| | | KTCYSIVEHNWFETFIVEMILLSSGALAFEDIYIEQRKTIKTMLEYADKVFT |
| | | YIFILEMLLKWVAYGFQTYFTNAWCWLDFLIVDVSLVSLVANALGYSELGAI |
| | | KSLRTLRALRPLRALSRFEGMRVVVNALVGAIPSIMNVLLVCLIFWLIFSIM |
| | | GVNLFAGKFYHCVNMTTGNMFDISDVNNLSDCQALGKQARWKNVKVNEDNVG |
| | | AGYLALLQVATFKGWMDIMYAAVDSRDVKLQPVYEENLYMYLYFVIFIIFGS |
| | | FFTLNLFIGVIIDNFNQQKKKFGGQDIFMTEEQKKYYNAMKKLGSKKPQKPI |
| | | PRPANKFQGMVFDFVTRQVEDISIMILICLNMVTMMVETDDQGKYMTLVLSR |
| | | INLVFIVLFTGEFVLKLVSLRHYYFTIGWNIFDFVVVILSIVGMFLAEMIEK |
| | | YFVSPTLFRVIRLARIGRILRLIKGAKGIRTLLFALMMSLPALFNIGLLLFL |
| | | VMFIYAIFGMSNFAYVKKEAGIDDMFNFETFGNSMICLFQITTSAGWDGLLA |
| | | PILNSAPPDCDPDTIHPGSSVKGDCGNPSVGIFFFVSYIIISFLVVVNMYIA |
| | | VILENFSVATEESAEPLSEDDFEMFYEVWEKFDPDATQFIEFSKLSDFAAAL |
| | | DPPLLIAKPNKVQLIAMDLPMVSGDRIHCLDILFAFTKRVLGESGEMDALRI |
| | | QMEDRFMASNPSKVSYEPITTTLKRKQEEVSAAIIQRNFRCYLLKQRLKNIS |
| | | SNYNKEAIKGRIDLPIKQDMIIDKLNGNSTPEKTDGSSSTTSPPSYDSVTKP |
| | | DKEKFEKDKPEKESKGKEVRENQK |
| 31 | Human Nav 1.4 | MARPSLCTLVPLGPECLRPFTRESLAAIEQRAVEEEARLQRNKQMEIEEPER |
| | (FIG. 9) | KPRSDLEAGKNLPMIYGDPPPEVIGIPLEDLDPYYSNKKTFIVLNKGKAIFR |
| | | FSATPALYLLSPFSVVRRGAIKVLIHALFSMFIMITILTNCVFMTMSDPPPW |
| | | SKNVEYTFTGIYTFESLIKILARGFCVDDFTFLRDPWNWLDFSVIMMAYLTE |
| | | FVDLGNISALRTFRVLRALKTITVIPGLKTIVGALIQSVKKLSDVMILTVFC |
| | | LSVFALVGLQLFMGNLRQKCVRWPPPENDTNTTWYSNDTWYGNDTWYGNEMW |
| | | YGNDSWYANDTWNSHASWATNDTFDWDAYISDEGNFYFLEGSNDALLCGNSS |
| | | DAGHCPEGYECIKTGRNPNYGYTSYDTFSWAFLALFRLMTQDYWENLFQLTL |
| | | RAAGKTYMIFFVVIIFLGSFYLINLILAVVAMAYAEQNEATLAEDKEKEEEF |
| | | QQMLEKFKKHQEELEKAKAAQALEGGEADGDPAHGKDCNGSLDTSQGEKGAP |
| | | RQSSSGDSGISDAMEELEEAHQKCPPWWYKCAHKVLIWNCCAPWLKFKNIIH |
| | | LIVMDPFVDLGITICIVLNTLFMAMEHYPMTEHFDNVLTVGNLVFTGIFTAE |
| | | MVLKLIAMDPYEYFQQGWNIFDSIIVTLSLVELGLANVQGLSVLRSFRLLRV |
| | | FKLAKSWPTLNMLIKIIGNSVGALGNLTLVLAIIVFIFAVVGMQLFGKSYKE |
| | | CVCKIALDCNLPRWHMHDFFHSFLIVFRILCGEWIETMWDCMEVAGQAMCLT |
| | | VFLMVMVIGNLVVLNLFLALLLSSFSADSLAASDEDGEMNNLQIAIGRIKLG |
| | | IGFAKAFLLGLLHGKILSPKDIMLSLGEADGAGEAGEAGETAPEDEKKEPPE |
| | | EDLKKDNHILNHMGLADGPPSSLELDHLNFINNPYLTIQVPIASEESDLEMP |
| | | TEEETDTFSEPEDSKKPWPCLYVDISQGRGKKWWTLRRACFKIVEHNWFETF |
| | | IVFMILLSSGALAFEDIYIEQRRVIRTILEYADKVFTYIFIMEMLLKWVAYG |
| | | FKVYFTNAWCWLDFLIVDVSIISLVANWLGYSELGPIKSLRTLRALRPLRAL |
| | | SRFEGMRVVVNALLGAIPSIMNVLLVCLIFWLIFSIMGVNLFAGKFYYCINT |
| | | TTSERFDISEVNNKSECESLMHTGQVRWLNVKVNYDNVGLGYLSLLQVATFK |
| | | GWMDIMYAAVDSREKEEQPQYEVNLYMYLYFVIFIIFGSFFTLNLFIGVIID |
| | | NFNQQKKKLGGKDIFMTEEQKKYYNAMKKLGSKKPQKPIPRPQNKIQGMVYD |
| | | LVTKQAFDITIMILICLNMVTMMVETDNQSQLKVDILYNINMIFIIIFTGEC |
| | | VLKMLALRQYYFTVGWNIFDFVVVILSIVGLALSDLIQKYFVSPTLFRVIRL |
| | | ARIGRVLRLIRGAKGIRTLLFALMMSLPALFNIGLLLFLVMFIYSIFGMSNF |
| | | AYVKKESGIDDMFNFETFGNSIICLFEITTSAGWDGLLNPILNSGPPDCDPN |
| | | LENPGTSVKGDCGNPSIGICFFCSYIIISFLIVVNMYIAIILENENVATEES |
| | | SEPLGEDDFEMFYETWEKFDPDATQFIAYSRLSDFVDTLQEPLRIAKPNKIK |
| | | LITLDLPMVPGDKIHCLDILFALTKEVLGDSGEMDALKQTMEEKFMAANPSK |
| | | VSYEPITTTLKRKHEEVCAIKIQRAYRRHLLQRSMKQASYMYRHSHDGSGDD |
| | | APEKEGLLANTMSKMYGHENGNSSSPSPEEKGEAGDAGPTMGLMPISPSDTA |
| | | WPPAPPPGQTVRPGVKESLV |
| 32 | Human Nav 1.5 | MANFLLPRGTSSFRRFTRESLAAIEKRMAEKQARGSTTLQESREGLPEEEAP |
| | (FIG. 9) | RPQLDLQASKKLPDLYGNPPQELIGEPLEDLDPFYSTQKTFIVLNKGKTIFR |
| | | FSATNALYVLSPFHPIRRAAVKILVHSLENMLIMCTILTNCVFMAQHDPPPW |
| | | TKYVEYTFTAIYTFESLVKILARGFCLHAFTFLRDPWNWLDESVIIMAYTTE |
| | | FVDLGNVSALRTFRVLRALKTISVISGLKTIVGALIQSVKKLADVMVLTVFC |
| | | LSVFALIGLQLFMGNLRHKCVRNFTALNGTNGSVEADGLVWESLDLYLSDPE |
| | | NYLLKNGTSDVLLCGNSSDAGTCPEGYRCLKAGENPDHGYTSFDSFAWAFLA |
| | | LFRLMTQDCWERLYQQTLRSAGKIYMIFFMLVIFLGSFYLVNLILAVVAMAY |
| | | EEQNQATIAETEEKEKRFQEAMEMLKKEHEALTIRGVDTVSRSSLEMSPLAP |
| | | VNSHERRSKRRKRMSSGTEECGEDRLPKSDSEDGPRAMNHLSLTRGLSRTSM |
| | | KPRSSRGSIFTFRRRDLGSEADFADDENSTAGESESHHTSLLVPWPLRRTSA |
| | | QGQPSPGTSAPGHALHGKKNSTVDCNGVVSLLGAGDPEATSPGSHLLRPVML |
| | | EHPPDTTTPSEEPGGPQMLTSQAPCVDGFEEPGARQRALSAVSVLTSALEEL |
| | | EESRHKCPPCWNRLAQRYLIWECCPLWMSIKQGVKLVVMDPFTDLTITMCIV |
| | | LNTLFMALEHYNMTSEFEEMLQVGNLVFTGIFTAEMTFKIIALDPYYYFQQG |
| | | WNIFDSIIVILSLMELGLSRMSNLSVLRSFRLLRVFKLAKSWPTLNTLIKII |
| | | GNSVGALGNLTLVLAIIVFIFAVVGMQLFGKNYSELRDSDSGLLPRWHMMDE |
| | | FHAFLIIFRILCGEWIETMWDCMEVSGQSLCLLVFLLVMVIGNLVVLNLFLA |
| | | LLLSSFSADNLTAPDEDREMNNLQLALARIQRGLRFVKRTTWDFCCGLLRQR |
| | | PQKPAALAAQGQLPSCIATPYSPPPPETEKVPPTRKETRFEEGEQPGQGTPG |
| | | DPEPVCVPIAVAESDTDDQEEDEENSLGTEEESSKQQESQPVSGGPEAPPDS |
| | | RTWSQVSATASSEAEASASQADWRQQWKAEPQAPGCGETPEDSCSEGSTADM |
| | | TNTAELLEQIPDLGQDVKDPEDCFTEGCVRRCPCCAVDTTQAPGKVWWRLRK |
| | | TCYHIVEHSWFETFIIFMILLSSGALAFEDIYLEERKTIKVLLEYADKMFTY |
| | | VFVLEMLLKWVAYGFKKYFTNAWCWLDFLIVDVSLVSLVANTLGFAEMGPIK |
| | | SLRTLRALRPLRALSRFEGMRVVVNALVGAIPSIMNVLLVCLIFWLIFSIMG |
| | | VNLFAGKFGRCINQTEGDLPLNYTIVNNKSQCESLNLTGELYWTKVKVNEDN |
| | | VGAGYLALLQVATFKGWMDIMYAAVDSRGYEEQPQWEYNLYMYIYFVIFIIF |
| | | GSFFTLNLFIGVIIDNFNQQKKKLGGQDIFMTEEQKKYYNAMKKLGSKKPQK |
| | | PIPRPLNKYQGFIFDIVTKQAFDVTIMFLICLNMVTMMVETDDQSPEKINIL |
| | | AKINLLEVAIFTGECIVKLAALRHYYFTNSWNIFDFVVVILSIVGTVLSDII |
| | | QKYFFSPTLFRVIRLARIGRILRLIRGAKGIRTLLFALMMSLPALFNIGLLL |
| | | FLVMFIYSIFGMANFAYVKWEAGIDDMFNFQTFANSMLCLFQITTSAGWDGL |
| | | LSPILNTGPPYCDPTLPNSNGSRGDCGSPAVGILFFTTYIIISFLIVVNMYI |
| | | AIILENFSVATEESTEPLSEDDFDMFYEIWEKFDPEATQFIEYSVLSDFADA |
| | | LSEPLRIAKPNQISLINMDLPMVSGDRIHCMDILFAFTKRVLGESGEMDALK |
| | | IQMEEKFMAANPSKISYEPITTTLRRKHEEVSAMVIQRAFRRHLLQRSLKHA |
| | | SFLFRQQAGSGLSEEDAPEREGLIAYVMSENFSRPLGPPSSSSISSTSFPPS |
| | | YDSVTRATSDNLQVRGSDYSHSEDLADFPPSPDRDRESIV |
| 33 | Human Nav 1.6 | MAARLLAPPGPDSFKPFTPESLANIERRIAESKLKKPPKADGSHREDDEDSK |
| | (FIG. 9) | PKPNSDLEAGKSLPFIYGDIPQGLVAVPLEDFDPYYLTQKTFVVLNRGKTLF |
| | | RFSATPALYILSPFNLIRRIAIKILIHSVFSMIIMCTILTNCVFMTFSNPPD |
| | | WSKNVEYTFTGIYTFESLVKIIARGFCIDGFTFLRDPWNWLDFSVIMMAYIT |
| | | EFVNLGNVSALRTFRVLRALKTISVIPGLKTIVGALIQSVKKLSDVMILTVE |
| | | CLSVFALIGLQLFMGNLRNKCVVWPINFNESYLENGTKGEDWEEYINNKTNF |
| | | YTVPGMLEPLLCGNSSDAGQCPEGYQCMKAGRNPNYGYTSFDTFSWAFLALF |
| | | RLMTQDYWENLYQLTLRAAGKTYMIFFVLVIFVGSFYLVNLILAVVAMAYEE |
| | | QNQATLEEAEQKEAEFKAMLEQLKKQQEEAQAAAMATSAGTVSEDAIEEEGE |
| | | EGGGSPRSSSEISKLSSKSAKERRNRRKKRKQKELSEGEEKGDPEKVFKSES |
| | | EDGMRRKAFRLPDNRIGRKESIMNQSLLSIPGSPFLSRHNSKSSIFSFRGPG |
| | | RFRDPGSENEFADDEHSTVEESEGRRDSLFIPIRARERRSSYSGYSGYSQGS |
| | | RSSRIFPSLRRSVKRNSTVDCNGVVSLIGGPGSHIGGRLLPEATTEVEIKKK |
| | | GPGSLLVSMDQLASYGRKDRINSIMSVVTNTLVEELEESQRKCPPCWYKFAN |
| | | TFLIWECHPYWIKLKEIVNLIVMDPFVDLAITICIVLNTLFMAMEHHPMTPQ |
| | | FEHVLAVGNLVFTGIFTAEMFLKLIAMDPYYYFQEGWNIFDGFIVSLSLMEL |
| | | SLADVEGLSVLRSFRLLRVFKLAKSWPTLNMLIKIIGNSVGALGNLTLVLAI |
| | | IVFIFAVVGMQLFGKSYKECVCKINQDCELPRWHMHDFFHSFLIVERVLCGE |
| | | WIETMWDCMEVAGQAMCLIVFMMVMVIGNLVVLNLFLALLLSSFSADNLAAT |
| | | DDDGEMNNLQISVIRIKKGVAWTKLKVHAFMQAHFKQREADEVKPLDELYEK |
| | | KANCIANHTGADIHRNGDFQKNGNGTTSGIGSSVEKYIIDEDHMSFINNPNL |
| | | TVRVPIAVGESDFENLNTEDVSSESDPEGSKDKLDDTSSSEGSTIDIKPEVE |
| | | EVPVEQPEEYLDPDACFTEGCVQRFKCCQVNIEEGLGKSWWILRKTCFLIVE |
| | | HNWFETFIIFMILLSSGALAFEDIYIEQRKTIRTILEYADKVETYIFILEML |
| | | LKWTAYGFVKFFTNAWCWLDFLIVAVSLVSLIANALGYSELGAIKSLRTLRA |
| | | LRPLRALSRFEGMRVVVNALVGAIPSIMNVLLVCLIFWLIFSIMGVNLFAGK |
| | | YHYCFNETSEIRFEIEDVNNKTECEKLMEGNNTEIRWKNVKINFDNVGAGYL |
| | | ALLQVATFKGWMDIMYAAVDSRKPDEQPKYEDNIYMYIYFVIFIIFGSFFTL |
| | | NLFIGVIIDNFNQQKKKFGGQDIFMTEEQKKYYNAMKKLGSKKPQKPIPRPL |
| | | NKIQGIVFDFVTQQAFDIVIMMLICLNMVTMMVETDTQSKQMENILYWINLV |
| | | FVIFFTCECVLKMFALRHYYFTIGWNIFDFVVVILSIVGMFLADIIEKYFVS |
| | | PTLFRVIRLARIGRILRLIKGAKGIRTLLFALMMSLPALFNIGLLLFLVMFI |
| | | FSIFGMSNFAYVKHEAGIDDMFNFETFGNSMICLFQITTSAGWDGLLLPILN |
| | | RPPDCSLDKEHPGSGFKGDCGNPSVGIFFFVSYIIISFLIVVNMYIAIILEN |
| | | FSVATEESADPLSEDDFETFYEIWEKFDPDATQFIEYCKLADFADALEHPLR |
| | | VPKPNTIELIAMDLPMVSGDRIHCLDILFAFTKRVLGDSGELDILRQQMEER |
| | | FVASNPSKVSYEPITTTLRRKQEEVSAVVLQRAYRGHLARRGFICKKTTSNK |
| | | LENGGTHREKKESTPSTASLPSYDSVTKPEKEKQQRAEEGRRERAKRQKEVR |
| | | ESKC |
| 34 | Human Nav 1.7 | MAMLPPPGPQSFVHFTKQSLALIEQRIAERKSKEPKEEKKDDDEEAPKPSSD |
| | (FIG. 9) | LEAGKQLPFIYGDIPPGMVSEPLEDLDPYYADKKTFIVLNKGKTIFRFNATP |
| | | ALYMLSPFSPLRRISIKILVHSLESMLIMCTILTNCIFMTMNNPPDWTKNVE |
| | | YTFTGIYTFESLVKILARGFCVGEFTFLRDPWNWLDFVVIVFAYLTEFVNLG |
| | | NVSALRTFRVLRALKTISVIPGLKTIVGALIQSVKKLSDVMILTVFCLSVFA |
| | | LIGLQLFMGNLKHKCFRNSLENNETLESIMNTLESEEDFRKYFYYLEGSKDA |
| | | LLCGESTDSGQCPEGYTCVKIGRNPDYGYTSFDTFSWAFLALFRLMTQDYWE |
| | | NLYQQTLRAAGKTYMIFFVVVIFLGSFYLINLILAVVAMAYEEQNQANIEEA |
| | | KQKELEFQQMLDRLKKEQEEAEAIAAAAAEYTSIRRSRIMGLSESSSETSKL |
| | | SSKSAKERRNRRKKKNQKKLSSGEEKGDAEKLSKSESEDSIRRKSFHLGVEG |
| | | HRRAHEKRLSTPNQSPLSIRGSLESARRSSRTSLFSFKGRGRDIGSETEFAD |
| | | DEHSIFGDNESRRGSLFVPHRPQERRSSNISQASRSPPMLPVNGKMHSAVDC |
| | | NGVVSLVDGRSALMLPNGQLLPEVIIDKATSDDSGTTNQIHKKRRCSSYLLS |
| | | EDMLNDPNLRQRAMSRASILTNTVEELEESRQKCPPWWYRFAHKFLIWNCSP |
| | | YWIKFKKCIYFIVMDPFVDLAITICIVLNTLFMAMEHHPMTEEFKNVLAIGN |
| | | LVFTGIFAAEMVLKLIAMDPYEYFQVGWNIFDSLIVTLSLVELFLADVEGLS |
| | | VLRSFRLLRVFKLAKSWPTLNMLIKIIGNSVGALGNLTLVLAIIVFIFAVVG |
| | | MQLFGKSYKECVCKINDDCTLPRWHMNDFFHSFLIVERVLCGEWIETMWDCM |
| | | EVAGQAMCLIVYMMVMVIGNLVVLNLFLALLLSSFSSDNLTAIEEDPDANNL |
| | | QIAVTRIKKGINYVKQTLREFILKAFSKKPKISREIRQAEDLNTKKENYISN |
| | | HTLAEMSKGHNFLKEKDKISGFGSSVDKHLMEDSDGQSFIHNPSLTVTVPIA |
| | | PGESDLENMNAEELSSDSDSEYSKVRLNRSSSSECSTVDNPLPGEGEEAEAE |
| | | PMNSDEPEACFTDGCVWRFSCCQVNIESGKGKIWWNIRKTCYKIVEHSWFES |
| | | FIVLMILLSSGALAFEDIYIERKKTIKIILEYADKIFTYIFILEMLLKWIAY |
| | | GYKTYFTNAWCWLDFLIVDVSLVTLVANTLGYSDLGPIKSLRTLRALRPLRA |
| | | LSRFEGMRVVVNALIGAIPSIMNVLLVCLIFWLIFSIMGVNLFAGKFYECIN |
| | | TTDGSRFPASQVPNRSECFALMNVSQNVRWKNLKVNFDNVGLGYLSLLQVAT |
| | | FKGWTIIMYAAVDSVNVDKQPKYEYSLYMYIYFVVFIIFGSFFTLNLFIGVI |
| | | IDNFNQQKKKLGGQDIFMTEEQKKYYNAMKKLGSKKPQKPIPRPGNKIQGCI |
| | | FDLVTNQAFDISIMVLICLNMVTMMVEKEGQSQHMTEVLYWINVVFIILFTG |
| | | ECVLKLISLRHYYFTVGWNIFDFVVVIISIVGMFLADLIETYFVSPTLFRVI |
| | | RLARIGRILRLVKGAKGIRTLLFALMMSLPALFNIGLLLFLVMFIYAIFGMS |
| | | NFAYVKKEDGINDMFNFETFGNSMICLFQITTSAGWDGLLAPILNSKPPDCD |
| | | PKKVHPGSSVEGDCGNPSVGIFYFVSYIIISFLVVVNMYIAVILENFSVATE |
| | | ESTEPLSEDDFEMFYEVWEKFDPDATQFIEFSKLSDFAAALDPPLLIAKPNK |
| | | VQLIAMDLPMVSGDRIHCLDILFAFTKRVLGESGEMDSLRSQMEERFMSANP |
| | | SKVSYEPITTTLKRKQEDVSATVIQRAYRRYRLRQNVKNISSIYIKDGDRDD |
| | | DLLNKKDMAFDNVNENSSPEKTDATSSTTSPPSYDSVTKPDKEKYEQDRTEK |
| | | EDKGKDSKESKK |
| 35 | Human Nav 1.8 | MEFPIGSLETNNFRRFTPESLVEIEKQIAAKQGTKKAREKHREQKDQEEKPR |
| | (FIG. 9) | PQLDLKACNQLPKFYGELPAELIGEPLEDLDPFYSTHRTFMVLNKGRTISRF |
| | | SATRALWLFSPENLIRRTAIKVSVHSWFSLFITVTILVNCVCMTRTDLPEKI |
| | | EYVFTVIYTFEALIKILARGFCLNEFTYLRDPWNWLDFSVITLAYVGTAIDL |
| | | RGISGLRTFRVLRALKTVSVIPGLKVIVGALIHSVKKLADVTILTIFCLSVF |
| | | ALVGLQLFKGNLKNKCVKNDMAVNETTNYSSHRKPDIYINKRGTSDPLLCGN |
| | | GSDSGHCPDGYICLKTSDNPDFNYTSFDSFAWAFLSLFRLMTQDSWERLYQQ |
| | | TLRTSGKIYMIFFVLVIFLGSFYLVNLILAVVTMAYEEQNQATTDEIEAKEK |
| | | KFQEALEMLRKEQEVLAALGIDTTSLHSHNGSPLTSKNASERRHRIKPRVSE |
| | | GSTEDNKSPRSDPYNQRRMSFLGLASGKRRASHGSVFHFRSPGRDISLPEGV |
| | | TDDGVFPGDHESHRGSLLLGGGAGQQGPLPRSPLPQPSNPDSRHGEDEHQPP |
| | | PTSELAPGAVDVSAFDAGQKKTFLSAEYLDEPFRAQRAMSVVSIITSVLEEL |
| | | EESEQKCPPCLTSLSQKYLIWDCCPMWVKLKTILFGLVTDPFAELTITLCIV |
| | | VNTIFMAMEHHGMSPTFEAMLQIGNIVFTIFFTAEMVFKIIAFDPYYYFQKK |
| | | WNIFDCIIVTVSLLELGVAKKGSLSVLRSFRLLRVFKLAKSWPTLNTLIKII |
| | | GNSVGALGNLTIILAIIVFVFALVGKQLLGENYRNNRKNISAPHEDWPRWHM |
| | | HDFFHSFLIVFRILCGEWIENMWACMEVGQKSICLILFLTVMVLGNLVVLNL |
| | | FIALLLNSFSADNLTAPEDDGEVNNLQVALARIQVFGHRTKQALCSFFSRSC |
| | | PFPQPKAEPELVVKLPLSSSKAENHIAANTARGSSGGLQAPRGPRDEHSDFI |
| | | ANPTVWVSVPIAEGESDLDDLEDDGGEDAQSFQQEVIPKGQQEQLQQVERCG |
| | | DHLTPRSPGTGTSSEDLAPSLGETWKDESVPQVPAEGVDDTSSSEGSTVDCL |
| | | DPEEILRKIPELADDLEEPDDCFTEGCIRHCPCCKLDTTKSPWDVGWQVRKT |
| | | CYRIVEHSWFESFIIFMILLSSGSLAFEDYYLDQKPTVKALLEYTDRVFTFI |
| | | FVFEMLLKWVAYGFKKYFTNAWCWLDFLIVNISLISLTAKILEYSEVAPIKA |
| | | LRTLRALRPLRALSRFEGMRVVVDALVGAIPSIMNVLLVCLIFWLIFSIMGV |
| | | NLFAGKFWRCINYTDGEFSLVPLSIVNNKSDCKIQNSTGSFFWVNVKVNEDN |
| | | VAMGYLALLQVATFKGWMDIMYAAVDSREVNMQPKWEDNVYMYLYFVIFIIF |
| | | GGFFTLNLFVGVIIDNFNQQKKKLGGQDIFMTEEQKKYYNAMKKLGSKKPQK |
| | | PIPRPLNKFQGFVFDIVTRQAFDITIMVLICLNMITMMVETDDQSEEKTKIL |
| | | GKINQFFVAVFTGECVMKMFALRQYYFTNGWNVFDFIVVVLSIASLIFSAIL |
| | | KSLQSYFSPTLERVIRLARIGRILRLIRAAKGIRTLLFALMMSLPALENIGL |
| | | LLFLVMFIYSIFGMSSFPHVRWEAGIDDMFNFQTFANSMLCLFQITTSAGWD |
| | | GLLSPILNTGPPYCDPNLPNSNGTRGDCGSPAVGIIFFTTYIIISFLIMVNM |
| | | YIAVILENENVATEESTEPLSEDDFDMFYETWEKFDPEATQFITFSALSDFA |
| | | DTLSGPLRIPKPNRNILIQMDLPLVPGDKIHCLDILFAFTKNVLGESGELDS |
| | | LKANMEEKFMATNLSKSSYEPIATTLRWKQEDISATVIQKAYRSYVLHRSMA |
| | | LSNTPCVPRAEEEAASLPDEGFVAFTANENCVLPDKSETASATSFPPSYESV |
| | | TRGLSDRVNMRTSSSIQNEDEATSMELIAPGP |
| 36 | Human Nav 1.9 | MDDRCYPVIFPDERNFRPFTSDSLAAIEKRIAIQKEKKKSKDQTGEVPQPRP |
| | (FIG. 9) | QLDLKASRKLPKLYGDIPRELIGKPLEDLDPFYRNHKTFMVLNRKRTIYRES |
| | | AKHALFIFGPFNSIRSLAIRVSVHSLFSMFIIGTVIINCVFMATGPAKNSNS |
| | | NNTDIAECVFTGIYIFEALIKILARGFILDEFSFLRDPWNWLDSIVIGIAIV |
| | | SYIPGITIKLLPLRTFRVFRALKAISVVSRLKVIVGALLRSVKKLVNVIILT |
| | | FFCLSIFALVGQQLFMGSLNLKCISRDCKNISNPEAYDHCFEKKENSPEFKM |
| | | CGIWMGNSACSIQYECKHTKINPDYNYTNFDNFGWSFLAMFRLMTQDSWEKL |
| | | YQQTLRTTGLYSVFFFIVVIFLGSFYLINLTLAVVTMAYEEQNKNVAAEIEA |
| | | KEKMFQEAQQLLKEEKEALVAMGIDRSSLTSLETSYFTPKKRKLFGNKKRKS |
| | | FFLRESGKDQPPGSDSDEDCQKKPQLLEQTKRLSQNLSLDHFDEHGDPLQRQ |
| | | RALSAVSILTITMKEQEKSQEPCLPCGENLASKYLVWNCCPQWLCVKKVLRT |
| | | VMTDPFTELAITICIIINTVFLAMEHHKMEASFEKMLNIGNLVFTSIFIAEM |
| | | CLKIIALDPYHYFRRGWNIFDSIVALLSFADVMNCVLQKRSWPFLRSFRVLR |
| | | VFKLAKSWPTLNTLIKIIGNSVGALGSLTVVLVIVIFIFSVVGMQLFGRSEN |
| | | SQKSPKLCNPTGPTVSCLRHWHMGDFWHSFLVVFRILCGEWIENMWECMQEA |
| | | NASSSLCVIVFILITVIGKLVVLNLFIALLLNSESNEERNGNLEGEARKTKV |
| | | QLALDRFRRAFCFVRHTLEHFCHKWCRKQNLPQQKEVAGGCAAQSKDIIPLV |
| | | MEMKRGSETQEELGILTSVPKTLGVRHDWTWLAPLAEEEDDVEFSGEDNAQR |
| | | ITQPEPEQQAYELHQENKKPTSQRVQSVEIDMFSEDEPHLTIQDPRKKSDVT |
| | | SILSECSTIDLQDGFGWLPEMVPKKQPERCLPKGFGCCFPCCSVDKRKPPWV |
| | | IWWNLRKTCYQIVKHSWFESFIIFVILLSSGALIFEDVHLENQPKIQELLNC |
| | | TDIIFTHIFILEMVLKWVAFGFGKYFTSAWCCLDFIIVIVSVTTLINLMELK |
| | | SFRTLRALRPLRALSQFEGMKVVVNALIGAIPAILNVLLVCLIFWLVFCILG |
| | | VYFFSGKFGKCINGTDSVINYTIITNKSQCESGNFSWINQKVNFDNVGNAYL |
| | | ALLQVATFKGWMDIIYAAVDSTEKEQQPEFESNSLGYIYFVVFIIFGSFFTL |
| | | NLFIGVIIDNFNQQQKKLGGQDIFMTEEQKKYYNAMKKLGSKKPQKPIPRPL |
| | | NKCQGLVEDIVTSQIFDIIIISLIILNMISMMAESYNQPKAMKSILDHLNWV |
| | | FVVIFTLECLIKIFALRQYYFTNGWNLFDCVVVLLSIVSTMISTLENQEHIP |
| | | FPPTLFRIVRLARIGRILRLVRAARGIRTLLFALMMSLPSLFNIGLLLFLIM |
| | | FIYAILGMNWFSKVNPESGIDDIFNFKTFASSMLCLFQISTSAGWDSLLSPM |
| | | LRSKESCNSSSENCHLPGIATSYFVSYIIISFLIVVNMYIAVILENENTATE |
| | | ESEDPLGEDDFDIFYEVWEKFDPEATQFIKYSALSDFADALPEPLRVAKPNK |
| | | YQFLVMDLPMVSEDRLHCMDILFAFTARVLGGSDGLDSMKAMMEEKFMEANP |
| | | LKKLYEPIVTTTKRKEEERGAAIIQKAFRKYMMKVTKGDQGDQNDLENGPHS |
| | | PLQTLCNGDLSSFGVAKGKVHCD |
| 37 | Human Nax 1204- | GGSNIFITVKQRKQYRRLKKLMYEDSQRPVPRPLNK |
| | 1239 | |
| | (FIG. 14B) | |
| 38 | Human Nav 1.1 | GGQDIFMTEEQKKYYNAMKKLGSKKPQKPIPRPGNK |
| | 1494-1529 | |
| | (FIG. 14B) | |
| 39 | Human Nav 1.2 | GGQDIFMTEEQKKYYNAMKKLGSKKPQKPIPRPANK |
| | 1484-1519 | |
| | (FIG. 14B) | |
| 40 | Human Nav 1.3 | GGQDIFMTEEQKKYYNAMKKLGSKKPQKPIPRPANK |
| | 1479-1514 | |
| | (FIG. 14B) | |
| 41 | Human Nav 1.4 | GGKDIFMTEEQKKYYNAMKKLGSKKPQKPIPRPQNK |
| | 1206-1241 | |
| | (FIG. 14B) | |
| 42 | Human Nav 1.5 | GGQDIFMTEEQKKYYNAMKKLGSKKPQKPIPRPLNK |
| | 1481-1516 | |
| | (FIG. 14B) | |
| 43 | Human Nav 1.6 | GGQDIFMTEEQKKYYNAMKKLGSKKPQKPIPRPLNK |
| | 1475-1510 | |
| | (FIG. 14B) | |
| 44 | Human Nav 1.7 | GGQDIFMTEEQKKYYNAMKKLGSKKPQKPIPRPGNK |
| | 1468-1503 | |
| | (FIG. 14B) | |
| 45 | Human Nav 1.8 | GGQDIFMTEEQKKYYNAMKKLGSKKPQKPIPRPLNK |
| | 1429-1464 | |
| | (FIG. 14B) | |
| 46 | Human Nav 1.9 | GGQDIFMTEEQKKYYNAMKKLGSKKPQKPIPRPLNK |
| | 1319-1354 | |
| | (FIG. 14B) | |
| 47 | Naₓ_D1_S6 (FIG. | GKVYMIFFVVVSFLFSFYMASLFLGILAMAY |
| | 20) | |
| 48 | Naᵥ1.1_D1_S6 | GKTYMIFFVLVIFLGSFYLINLILAVVAMAY |
| | (FIG. 20) | |
| 49 | Naᵥ1.2_D1_S6 | GKTYMIFFVLVIFLGSFYLINLILAVVAMAY |
| | (FIG. 20) | |
| 50 | Naᵥ1.3_D1_S6 | GKTYMIFFVLVIFLGSFYLVNLILAVVAMAY |
| | (FIG. 20) | |
| 51 | Naᵥ1.4_D1_S6 | GKTYMIFFVVIIFLGSFYLINLILAVVAMAY |
| | (FIG. 20) | |
| 52 | Na_{y}1.5_D1_S6 | GKIYMIFFMLVIFLGSFYLVNLILAVVAMAY |
| | (FIG. 20) | |
| 53 | Naᵥ1.6_D1_S6 | GKTYMIFFVLVIFVGSFYLVNLILAVVAMAY |
| | (FIG. 20) | |
| 54 | Naᵥ1.7_D1_S6 | GKTYMIFFVVVIFLGSFYLINLILAVVAMAY |
| | (FIG. 20) | |
| 55 | Naᵥ1.8_D1_S6 | GKIYMIFFVLVIFLGSFYLVNLILAVVTMAY |
| | (FIG. 20) | |
| 56 | Naᵥ1.9_D1_S6 | GLYSVFFFIVVIFLGSFYLINLTLAVVTMAY |
| | (FIG. 20) | |
| 57 | Naₓ_D2_S6 (FIG. | QSWCIPFYLMVILIGNLLVLYLFLALVSSF |
| | 20) | |
| 58 | Naᵥ1.1_D2_S6 | QAMCLTVFMMVMVIGNLVVLNLFLALLLSSF |
| | (FIG. 20) | |
| 59 | Naᵥ1.2_D2_S6 | QTMCLTVFMMVMVIGNLVVLNLFLALLLSSF |
| | (FIG. 20) | |
| 60 | Naᵥ1.3_D2_S6 | QTMCLIVFMLVMVIGNLVVLNLFLALLLSSF |
| | (FIG. 20) | |
| 61 | Naᵥ1.4_D2_S6 | QAMCLTVFLMVMVIGNLVVLNLFLALLLSSF |
| | (FIG. 20) | |
| 62 | Naᵥ1.5_D2_S6 | QSLCLLVFLLVMVIGNLVVLNLFLALLLSSF |
| | (FIG. 20) | |
| 63 | Naᵥ1.6_D2_S6 | QAMCLIVFMMVMVIGNLVVLNLFLALLLSSF |
| | (FIG. 20) | |
| 64 | Naᵥ1.7_D2_S6 | QAMCLIVYMMVMVIGNLVVLNLFLALLLSSF |
| | (FIG. 20) | |
| 65 | Naᵥ1.8_D2_S6 | KSICLILFLTVMVLGNLVVLNLFIALLLNSF |
| | (FIG. 20) | |
| 66 | Naᵥ1.9_D2_S6 | SSLCVIVFILITVIGKLVVLNLFIALLLNSF |
| | (FIG. 20) | |
| 67 | Naₓ_D3_S6 (FIG. | NIYMYCYFINFIIFGVFLPLSMLITVIIDNF |
| | 20) | |
| 68 | Naᵥ1.1_D3_S6 | SLYMYLYFVIFIIFGSFFTLNLFIGVIIDNF |
| | (FIG. 20) | |
| 69 | Naᵥ1.2_D3_S6 | NLYMYLYFVIFIIFGSFFTLNLFIGVIIDNF |
| | (FIG. 20) | |
| 70 | Naᵥ1.3_D3_S6 | NLYMYLYFVIFIIFGSFFTLNLFIGVIIDNF |
| | (FIG. 20) | |
| 71 | Naᵥ1.4_D3_S6 | NLYMYLYFVIFIIFGSFFTLNLFIGVIIDNE |
| | (FIG. 20) | |
| 72 | Naᵥ1.5_D3_S6 | NLYMYIYFVIFIIFGSFFTLNLFIGVIIDNF |
| | (FIG. 20) | |
| 73 | Naᵥ1.6_D3_S6 | NIYMYIYFVIFIIFGSFFTLNLFIGVIIDNF |
| | (FIG. 20) | |
| 74 | Naᵥ1.7_D3_S6 | SLYMYIYFVVFIIFGSFFTLNLFIGVIIDNE |
| | (FIG. 20) | |
| 75 | Naᵥ1.8_D3_S6 | NVYMYLYFVIFIIFGGFFTLNLFVGVIIDNF |
| | (FIG. 20) | |
| 76 | Naᵥ1.9_D3_S6 | NSLGYIYFVVFIIFGSFFTLNLFIGVIIDNF |
| | (FIG. 20) | |
| 77 | Naₓ_D4_S6 | PSVGIFYFVSYILISWLIIVNMYIVVVMEFL |
| | (FIG. 20) | |
| 78 | Naᵥ1.1_D4_S6 | PSVGIFFFVSYIIISFLVVVNMYIAVILENF |
| | (FIG. 20) | |
| 79 | Naᵥ1.2_D4_S6 | PSVGIFFFVSYIIISFLVVVNMYIAVILENF |
| | (FIG. 20) | |
| 80 | Naᵥ1.3_D4_S6 | PSVGIFFFVSYIIISFLVVVNMYIAVILENF |
| | (FIG. 20) | |
| 81 | Naᵥ1.4_D4_S6 | PSIGICFFCSYIIISFLIVVNMYIAIILENE |
| | (FIG. 20) | |
| 82 | Naᵥ1.5_D4_S6 | PAVGILFFTTYIIISFLIVVNMYIAIILENF |
| | (FIG. 20) | |
| 83 | Naᵥ1.6_D4_S6 | PSVGIFFFVSYIIISFLIVVNMYIAIILENF |
| | (FIG. 20) | |
| 84 | Naᵥ1.7_D4_S6 | PSVGIFYFVSYIIISFLVVVNMYIAVILENF |
| | (FIG. 20) | |
| 85 | Naᵥ1.8_D4_S6 | PAVGIIFFTTYIIISFLIMVNMYIAVILENF |
| | (FIG. 20) | |
| 86 | Naᵥ1.9_D4_S6 | PGIATSYFVSYIIISFLIVVNMYIAVILENF |
| | (FIG. 20) | |
| 87 | Naₓ VSLD1 | PTLQTARTLRILKIIP |
| 88 | Naₓ VSLD2 | ALLRLFRMLRIFKLGK |
| 89 | Naₓ VSLD3 | KPLISMKFLRPLRVLSQ |
| 90 | Naₓ VSLD4 | QLILLSRIIHMLRLGKGPKVFH |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of identifying a human Na_{X} ion channel protein (Na_{X}) modulator, comprising:
a) Providing a mutant human Na_{X} wherein at least a portion of the DIII domain of human Na_{X} is replaced by a corresponding portion of the DIII domain of a human or mammalian Na_{V} protein (Na_{V}) and/or comprising a substitution of at least one residue on each of two, three, or four S6 alpha helices of human Na_{X} with a glycine, proline, or polar or charged residue;
b) Performing an ion channel assay on the mutant human Na_{X} in the presence of a potential Na_{X} modulator; and
c) Identifying the potential modulator as a human Na_{X} modulator if the activity of the mutant human Na_{X} in the assay in the presence of the potential modulator is higher or lower than the activity in the absence of the potential modulator.

2. The method of claim 1, wherein the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237).

3. The method of claim 1, wherein the at least a portion of the DIII domain of human Na_{X} that is replaced by a corresponding portion of the DIII domain of the human or mammalian Na_{V} (a) does not comprise S5 and/or does not comprise S6 or (b) does not comprise any of S1, S2, S3, S4, and/or S4-S5 linker.

4. (canceled)

5. The method of claim 1, wherein the at least a portion of the DIII domain of human Na_{X} is replaced by a corresponding portion of mammalian Nav1.1, mammalian Nav1.2, mammalian Nav1.3, mammalian Nav1.4, mammalian Nav1.5, mammalian Nav1.6, mammalian Nav1.7, mammalian Nav1.8, mammalian Nav1.9, human Nav1.1, human Nav1.2, human Nav1.3, human Nav1.4, human Nav1.5, human Nav1.6, human Nav1.7, human Nav1.8, or human Nav1.9.

6. (canceled)

7. The method of claim 6, wherein the mutant human Na_{X} comprises the amino acid sequence of one of SEQ ID Nos: 3, 4, 8, 10, 12, 16, or 20.

8. (canceled)

9. The method of claim 1, wherein the mutant human Na_{X} comprises a substitution of at least one residue on two, three, or all four of the four S6 alpha helices with a glycine, proline, or polar or charged residue.

10. (canceled)

11. (canceled)

12. (canceled)

13. The method of claim 1, wherein the mutant human Na_{X} comprises one or two substitutions of an amino acid residue with a glycine, proline, or polar or charged residue within at least two, within at least three, or within each of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV).

14. (canceled)

15. (canceled)

16. The method of claim 1, wherein the mutant human Na_{X} comprises substitutions of an amino acid residue at two or more or three or more of residues L390, F724, I1189, and I1492 with a glycine, proline, or polar or charged residue.

17. (canceled)

18. The method of claim 16, wherein the mutant human Na_{X} (a) comprises substitutions of amino acid residues L390, F724, and I1189 or of amino acid residues F724, I1189, and I1492 with glycine, proline, or polar or charged residues; (b) comprises substitutions F724Q, I1189T, and I1492T compared to wild-type human Na_{X}; or (c) comprises substitutions L390E, I1189E, and I1492E compared to wild-type human Na_{X}.

19. (canceled)

20. (canceled)

21. (canceled)

22. (canceled)

23. (canceled)

24. (canceled)

25. (canceled)

26. (canceled)

27. The method of claim 1, wherein the ion channel assay is a patch clamp or an automated patch clamp assay, an ion flux assay, or an ion- or voltage-sensitive dye assay.

28. (canceled)

29. (canceled)

30. (canceled)

31. (canceled)

32. A mutant human Na_{X} ion channel (Na_{X}) protein, wherein at least a portion of the DIII domain of human Na_{X} is replaced by a corresponding portion of the DIII domain of a human or mammalian Na_{V} protein (Na_{V}) and/or comprising a substitution of at least one residue on each of the two, three, or four S6 alpha helices with a glycine, proline, or polar or charged residue.

33. The mutant human Na_{X} of claim 32, wherein the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1137), (f) DIII and DII-DIII linker and DIII-DIV linker (733-1237).

34. The mutant human Na_{X} of claim 32, wherein the at least a portion of the DIII domain of human Na_{X} that is replaced by a corresponding portion of the DIII domain of the human or mammalian Na_{V} (a) does not comprise S5 and/or does not comprise S6; or (b) does not comprise any of S1, S2, S3, S4, and/or S4-S5 linker.

35. (canceled)

36. The mutant human Na_{X} of claim 32, wherein the at least a portion of the DIII domain of human Na_{X} is replaced by a corresponding portion of mammalian Nav1.1, mammalian Nav1.2, mammalian Nav1.3, mammalian Nav1.4, mammalian Nav1.5, mammalian Nav1.6, mammalian Nav1.7, mammalian Nav1.8, mammalian Nav1.9, human Nav1.1, human Nav1.2, human Nav1.3, human Nav1.4, human Nav1.5, human Nav1.6, human Nav1.7, human Nav1.8, or human Nav1.9.

37. (canceled)

38. The mutant human Na_{X} of claim 36, wherein the mutant human Na_{X} comprises the amino acid sequence of one of SEQ ID Nos: 3, 4, 8, 10, 12, 16, or 20.

39. (canceled)

40. (canceled)

41. (canceled)

42. (canceled)

43. (canceled)

44. The mutant human Na_{X} of claim 32, wherein the mutant human Na_{X} comprises one or two substitutions of an amino acid residue with a glycine, proline, or polar or charged residue within at least two or at least three, or each of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV).

45. (canceled)

46. (canceled)

47. The mutant human Na_{X} of claim 32, wherein the mutant human Na_{X} (a) comprises substitutions of an amino acid residue at two or more of residues L390, F724, I1189, and I1492 with a glycine, proline, or polar or charged residue; (b) comprises substitutions of an amino acid residue at three or more of residues L390, F724, I1189, and I1492 with a glycine, proline, or polar or charged residue; (c) comprises substitutions of amino acid residues L390, F724, and I1189 or of amino acid residues F724, I1189, and I1492 with glycine, proline, or polar or charged residues; (d) comprises substitutions of amino acid residues L390, F724, and I1189 or of amino acid residues F724, I1189, and I1492 with glycine, proline, or polar or charged residues; or (e) comprises substitutions L390E, I1189E, and I1492E compared to wild-type human Na_{X}.

48. (canceled)

49. (canceled)

50. (canceled)

51. (canceled)

52. A method of determining whether a test molecule that modulates the activity of a human ion channel protein modulates the activity of human Na_{X} ion channel (Na_{X}) protein, comprising:
a) Providing a mutant human Na_{X} in which at least a portion of the DIII domain of human Na_{X} is replaced by a corresponding portion of the DIII domain of a human or mammalian Na_{V} protein (Na_{V}) and/or comprising a substitution of at least one residue on each of two, three, or four S6 alpha helices of human Na_{X} with a glycine, proline, or polar or charged residue;
b) Performing an ion channel assay on the mutant human Na_{X} in the presence of the test molecule; and
c) Determining that the test molecule is a human Na_{X} modulator if the activity of the mutant human Na_{X} in the assay in the presence of the test molecule is higher or lower than the activity in the absence of the test molecule; and optionally,
d) Selecting the test molecule for additional screening if it is not a human Na_{X} modulator according to part (c).

53. (canceled)

54. The method of claim 52 or 53, wherein the mutant human Na_{X} comprises all or a portion of the DIII S1-S6 region from a human or mammalian Na_{V}, optionally wherein the at least a portion of the DIII domain of human Na_{X} that is replaced comprises or consists of: (a) DIII (residues 920-1200), (b) DIII voltage-sensor domain III (VSD3) and S4-S5 linker (residues 920-1058), (c) DIII VSD3, S4-S5 linker and S5 (residues 920-1078), (d) DIII and DII-DIII linker (residues 733-1200), (e) DIII and DIII-DIV linker (920-1237), (f) DIII and DII-DIII linker and DIII-DIV linker (733-1237).

55. The method of claim 52, wherein the at least a portion of the DIII domain of human Na_{X} that is replaced by a corresponding portion of the DIII domain of the human or mammalian Na_{V} (a) does not comprise S5 and/or does not comprise S6 or (b) does not comprise any of S1, S2, S3, S4, and/or S4-S5 linker.

56. (canceled)

57. The method of claim 52, wherein the at least a portion of the DIII domain of human Na_{X} is replaced by a corresponding portion of mammalian Nav1.1, mammalian Nav1.2, mammalian Nav1.3, mammalian Nav1.4, mammalian Nav1.5, mammalian Nav1.6, mammalian Nav1.7, mammalian Nav1.8, mammalian Nav1.9, human Nav1.1, human Nav1.2, human Nav1.3, human Nav1.4, human Nav1.5, human Nav1.6, human Nav1.7, human Nav1.8, or human Nav1.9.

58. (canceled)

59. The method of claim 52, wherein the mutant human Na_{X} comprises the amino acid sequence of one of SEQ ID Nos: 3, 4, 8, 10, 12, 16, or 20.

60. (canceled)

61. (canceled)

62. The method of claim 52, wherein the mutant human Na_{X} comprises a substitution of at least one residue on two, three, or all of the four S6 alpha helices with a glycine, proline, or polar or charged residue.

63. (canceled)

64. (canceled)

65. (canceled)

66. The method of claim 52, wherein the mutant human Na_{X} comprises one or two substitutions of an amino acid residue with a glycine, proline, or polar or charged residue within at least two, at least three, or within each of following segments of SEQ ID NO: 1: residues 383-397 (in S6 of domain I (D1)), residues 717-731 (in S6 of DII), residues 1182-1196 (in S6 of DIII), and/or residues 1485-1499 (in S6 of DIV).

67. (canceled)

68. (canceled)

69. The method of claim 52, wherein the mutant human Na_{X} comprises substitutions of an amino acid residue at two or more or three or more of residues L390, F724, I1189, and I1492 with a glycine, proline, or polar or charged residue.

70. (canceled)

71. The method of claim 52, wherein the mutant human Na_{X} (a) comprises substitutions of amino acid residues L390, F724, and I1189 or of amino acid residues F724, I1189, and I1492 with glycine, proline, or polar or charged residues; (b) comprises substitutions F724Q, I1189T, and I1492T compared to wild-type human Na_{X}; or (c) comprises substitutions L390E, I1189E, and I1492E compared to wild-type human Na_{X}.

72. (canceled)

73. (canceled)

74. (canceled)

75. (canceled)

76. A kit for assaying the activity of human Na_{X} or for identifying a human Na_{X} modulator and/or a molecule that binds to human Na_{X}, comprising the mutant human Na_{X} according to claim 39, and further comprising at least one of: one or more reagents for conducting an ion channel assay, a modulator of the mutant human Na_{X}, and instructions for use.


SEQUENCE LISTING (90 sequences)

SEQ ID NO: 1 - protein, 1682 aa, Homo sapiens
  REGION 1..1682: note = misc_feature - Wild-type human NaX (hNaX) (Uniprot Q01118-1)
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 2 - protein, 1940 aa, synthetic construct
  REGION 1..1940: note = Synthetic: hNaX_hNaV1.7_DI-DII_chimera
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 3 - protein, 1730 aa, synthetic construct
  REGION 1..1730: note = Synthetic: hNaX_hNaV1.7_DIII-DIV_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 4 - protein, 1707 aa, synthetic construct
  REGION 1..1707: note = Synthetic: hNaX_hNaV1.7_DII-DIII_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 5 - protein, 1963 aa, synthetic construct
  REGION 1..1963: note = Synthetic: hNaX_hNaV1.7_DI-DIV_chimera
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 6 - protein, 1921 aa, synthetic construct
  REGION 1..1921: note = Synthetic: hNaX-hNaV1.7_DI_chimera
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 7 - protein, 1701 aa, synthetic construct
  REGION 1..1701: note = Synthetic: hNaX-hNaV1.7_DII_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 8 - protein, 1688 aa, synthetic construct
  REGION 1..1688: note = Synthetic: hNaX-hNaV1.7_DIII_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 9 - protein, 1724 aa, synthetic construct
  REGION 1..1724: note = Synthetic: hNaX-hNaV1.7_DIV-DIII-DIVlinker_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 10 - protein, 1688 aa, synthetic construct
  REGION 1..1688: note = Synthetic: hNaX_hNaV1.7_DIII_Chimera_SF_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFSWAFLALFRLMTQDYWENLYQQTLRASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLIVFRVLCGEWIETMWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMICLFQITTSAGWDGLLAPILNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 11 - protein, 1688 aa, synthetic construct
  REGION 1..1688: note = Synthetic: hNaX_hNaV1.7_D3-D4link_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 12 - protein, 1687 aa, synthetic construct
  REGION 1..1687: note = Synthetic: hNaX_hNaV1.7_D3-D4link-PM_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAILPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 13 - protein, 1687 aa, synthetic construct
  REGION 1..1687: note = Synthetic: hNaX_hNaV1.7_D3-D4link-PM-S4S5link_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 14 - protein, 1687 aa, synthetic construct
  REGION 1..1687: note = Synthetic: hNaX_hNaV1.7_VSD3_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 15 - protein, 1687 aa, synthetic construct
  REGION 1..1687: note = Synthetic: hNaX_hNaV1.7_VSD3-S4S5link_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 16 - protein, 1688 aa, synthetic construct
  REGION 1..1688: note = Synthetic: hNaX_hNaV1.7_VSD3-S4S5link-PM_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 17 - protein, 1687 aa, synthetic construct
  REGION 1..1687: note = Synthetic: hNaX_hNaV1.7_DIII_VSD3-S3-S4_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFTNAWCWLDFLIVDVSLVTLVANTLGYSDLGPIKSLRTLRALRPLRALSRFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 18 - protein, 1683 aa, synthetic construct
  REGION 1..1683: note = Synthetic: hNaX_hNaV1.7_DIII_VSD3-S5-S6_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 19 - protein, 1683 aa, synthetic construct
  REGION 1..1683: note = Synthetic: hNaX_hNaV1.7_DIII_VSD3-S3-S4loop_S4-S5link_S5_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGGYSDLGLKPLISMKFLRPLRVLSQFEGMRVVVNALIGAIPSIMNVLLVCLIFWLIFSIMGVNLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 20 - protein, 1730 aa, synthetic construct
  REGION 1..1730: note = Synthetic: hNaX_hNaV1.7_DIII_DIVS6-CTD_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 21 - protein, 1876 aa, synthetic construct
  REGION 1..1876: note = Synthetic: NaX_NaV1.7_D1-D2_linker_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 22 - protein, 1701 aa, synthetic construct
  REGION 1..1701: note = Synthetic: NaX_NaV1.7_D2-D3_linker_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 23 - protein, 1669 aa, synthetic construct
  REGION 1..1669: note = Synthetic: NaX_NaV1.7_D3-D4_linker_chimera
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLILGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 24 - protein, 1911 aa, synthetic construct
  REGION 1..1911: note = Synthetic: NaX_NaV1.7_ultimateloop_chimera
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLILGGQDIFMTEEQKKYYNAMKKLGSKKPQKPIPRPGNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 25 - protein, 1682 aa, synthetic construct
  REGION 1..1682: note = Synthetic: NaX_QTT
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLQLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLTTVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYTVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 26 - protein, 1682 aa, synthetic construct
  REGION 1..1682: note = Synthetic: NaX_EEE
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFEGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLETVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYEVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 27 - protein, 1682 aa, Homo sapiens; identical to SEQ ID NO: 1
  REGION 1..1682: note = misc_feature - Human Nax (Figure 9)

SEQ ID NO: 28 - protein, 2009 aa, Homo sapiens
  REGION 1..2009: note = misc_feature - Human Nav 1.1 (Figure 9)
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 29 - protein, 2005 aa, Homo sapiens
  REGION 1..2005: note = misc_feature - Human Nav 1.2 (Figure 9)
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 30 - protein, 2000 aa, Homo sapiens
  REGION 1..2000: note = misc_feature - Human Nav 1.3 (Figure 9)
MAQALLVPPGPESFRLFTRESLAAIEKRAAEEKAKKPKKEQDNDDENKPKPNSDLEAGKNLPFIYGDIPPEMVSEPLEDLDPYYINKKTFIVMNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVSLGNVSALRTFRVLRALKTISVIPGLKTIVGALIQSVKKLSDVMILTVFCLSVFALIGLQLFMGNLRNKCLQWPPSDSAFETNTTSYFNGTMDSNGTFVNVTMSTFNWKDYIGDDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGIGGLGELLESSSEASKLSSKSAKEWRNRRKKRRQREHLEGNNKGERDSFPKSESEDSVKRSSFLFSMDGNRLTSDKKFCSPHQSLLSIRGSLFSPRRNSKTSIFSFRGRAKDVGSENDFADDEHSTFEDSESRRDSLFVPHRHGERRNSNVSQASMSSRMVPGLPANGKMHSTVDCNGVVSLVGGPSALTSPTGQLPPEGTTTETEVRKRRLSSYQISMEMLEDSSGRQRAVSIASILTNTMEELEESRQKCPPCWYRFANVFLIWDCCDAWLKVKHLVNLIVMDPFVDLAITICIVLNTLFMAMEHYPMTEQFSSVLTVGNLVFTGIFTAEMVLKIIAMDPYYYFQEGWNIFDGIIVSLSLMELGLSNVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKMRECFQKAFFRKPKVIEIHEGNKIDSCMSNNTGIEISKELNYLRDGNGTTSGVGTGSSVEKYVIDENDYMSFINNPSLTVTVPIAVGESDFENLNTEEFSSESELEESKEKLNATSSSEGSTVDVVLPREGEQAETEPEEDLKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNMTTGNMFDISDVNNLSDCQALGKQARWKNVKVNFDNVGAGYLALLQVATFKGWMDIMYAAVDSRDVKLQPVYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQGKYMTLVLSRINLVFIVLFTGEFVLKLVSLRHYYFTIGWNIFDFVVVILSIVGMFLAEMIEKYFVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDTIHPGSSVKGDCGNPSVGIFFFVSYIIISFLVVVNMYIAVILENFSVATEESAEPLSEDDFEMFYEVWEKFDPDATQFIEFSKLSDFAAALDPPLLIAKPNKVQLIAMDLPMVSGDRIHCLDILFAFTKRVLGESGEMDALRIQMEDRFMASNPSKVSYEPITTTLKRKQEEVSAAIIQRNFRCYLLKQRLKNISSNYNKEAIKGRIDLPIKQDMIIDKLNGNSTPEKTDGSSSTTSPPSYDSVTKPDKEKFEKDKPEKESKGKEVRENQK

SEQ ID NO: 31 - protein, 1788 aa, Homo sapiens
  REGION 1..1788: note = misc_feature - Human Nav 1.4 (Figure 9)
MARPSLCTLVPLGPECLRPFTRESLAAIEQRAVEEEARLQRNKQMEIEEPERKPRSDLEAGKNLPMIYGDPPPEVIGIPLEDLDPYYSNKKTFIVLNKGKAIFRFSATPALYLLSPFSVVRRGAIKVLIHALFSMFIMITILTNCVFMTMSDPPPWSKNVEYTFTGIYTFESLIKILARGFCVDDFTFLRDPWNWLDFSVIMMAYLTEFVDLGNISALRTFRVLRALKTITVIPGLKTIVGALIQSVKKLSDVMILTVFCLSVFALVGLQLFMGNLRQKCVRWPPPFNDTNTTWYSNDTWYGNDTWYGNEMWYGNDSWYANDTWNSHASWATNDTFDWDAYISDEGNFYFLEGSNDALLCGNSSDAGHCPEGYECIKTGRNPNYGYTSYDTFSWAFLALFRLMTQDYWENLFQLTLRAAGKTYMIFFVVIIFLGSFYLINLILAVVAMAYAEQNEATLAEDKEKEEEFQQMLEKFKKHQEELEKAKAAQALEGGEADGDPAHGKDCNGSLDTSQGEKGAPRQSSSGDSGISDAMEELEEAHQKCPPWWYKCAHKVLIWNCCAPWLKFKNIIHLIVMDPFVDLGITICIVLNTLFMAMEHYPMTEHFDNVLTVGNLVFTGIFTAEMVLKLIAMDPYEYFQQGWNIFDSIIVTLSLVELGLANVQGLSVLRSFRLLRVFKLAKSWPTLNMLIKIIGNSVGALGNLTLVLAIIVFIFAVVGMQLFGKSYKECVCKIALDCNLPRWHMHDFFHSFLIVFRILCGEWIETMWDCMEVAGQAMCLTVFLMVMVIGNLVVLNLFLALLLSSFSADSLAASDEDGEMNNLQIAIGRIKLGIGFAKAFLLGLLHGKILSPKDIMLSLGEADGAGEAGEAGETAPEDEKKEPPEEDLKKDNHILNHMGLADGPPSSLELDHLNFINNPYLTIQVPIASEESDLEMPTEEETDTFSEPEDSKKPWPCLYVDISQGRGKKWWTLRRACFKIVEHNWFETFIVFMILLSSGALAFEDIYIEQRRVIRTILEYADKVFTYIFIMEMLLKWVAYGFKVYFTNAWCWLDFLIVDVSIISLVANWLGYSELGPIKSLRTLRALRPLRALSRFEGMRVVVNALLGAIPSIMNVLLVCLIFWLIFSIMGVNLFAGKFYYCINTTTSERFDISEVNNKSECESLMHTGQVRWLNVKVNYDNVGLGYLSLLQVATFKGWMDIMYAAVDSREKEEQPQYEVNLYMYLYFVIFIIFGSFFTLNLFIGVIIDNFNQQKKKLGGKDIFMTEEQKKYYNAMKKLGSKKPQKPIPRPQNKIQGMVYDLVTKQAFDITIMILICLNMVTMMVETDNQSQLKVDILYNINMIFIIIFTGECVLKMLALRQYYFTVGWNIFDFVVVILSIVGLALSDLIQKYFVSPTLFRVIRLARIGRVLRLIRGAKGIRTLLFALMMSLPALFNIGLLLFLVMFIYSIFGMSNFAYVKKESGIDDMFNFETFGNSIICLFEITTSAGWDGLLNPILNSGPPDCDPNLENPGTSVKGDCGNPSIGICFFCSYIIISFLIVVNMYIAIILENFNVATEESSEPLGEDDFEMFYETWEKFDPDATQFIAYSRLSDFVDTLQEPLRIAKPNKIKLITLDLPMVPGDKIHCLDILFALTKEVLGDSGEMDALKQTMEEKFMAANPSKVSYEPITTTLKRKHEEVCAIKIQRAYRRHLLQRSMKQASYMYRHSHDGSGDDAPEKEGLLANTMSKMYGHENGNSSSPSPEEKGEAGDAGPTMGLMPISPSDTAWPPAPPPGQTVRPGVKESLV

SEQ ID NO: 32 - protein, 2016 aa, Homo sapiens [sequence omitted]

SEQ ID NO: 33 - protein, 1980 aa, Homo sapiens
  REGION 1..1980: note = misc_feature - Human Nav 1.6 (Figure 9)
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 34 - protein, 1988 aa, Homo sapiens
  REGION 1..1988: note = misc_feature - Human Nav 1.7 (Figure 9)
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 35 - protein, 1956 aa, Homo sapiens
  REGION 1..1956: note = misc_feature - Human Nav 1.8 (Figure 9)
MEFPIGSLETNNFRRFTPESLVEIEKQIAAKQGTKKAREKHREQKDQEEKPRPQLDLKACNQLPKFYGELPAELIGEPLEDLDPFYSTHRTFMVLNKGRTISRFSATRALWLFSPFNLIRRTAIKVSVHSWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALIHSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGKIYMIFFVLVIFLGSFYLVNLILAVVTMAYEEQNQATTDEIEAKEKKFQEALEMLRKEQEVLAALGIDTTSLHSHNGSPLTSKNASERRHRIKPRVSEGSTEDNKSPRSDPYNQRRMSFLGLASGKRRASHGSVFHFRSPGRDISLPEGVTDDGVFPGDHESHRGSLLLGGGAGQQGPLPRSPLPQPSNPDSRHGEDEHQPPPTSELAPGAVDVSAFDAGQKKTFLSAEYLDEPFRAQRAMSVVSIITSVLEELEESEQKCPPCLTSLSQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRTKQALCSFFSRSCPFPQPKAEPELVVKLPLSSSKAENHIAANTARGSSGGLQAPRGPRDEHSDFIANPTVWVSVPIAEGESDLDDLEDDGGEDAQSFQQEVIPKGQQEQLQQVERCGDHLTPRSPGTGTSSEDLAPSLGETWKDESVPQVPAEGVDDTSSSEGSTVDCLDPEEILRKIPELADDLEEPDDCFTEGCIRHCPCCKLDTTKSPWDVGWQVRKTCYRIVEHSWFESFIIFMILLSSGSLAFEDYYLDQKPTVKALLEYTDRVFTFIFVFEMLLKWVAYGFKKYFTNAWCWLDFLIVNISLISLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTKILGKINQFFVAVFTGECVMKMFALRQYYFTNGWNVFDFIVVVLSIASLIFSAILKSLQSYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIMVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTPCVPRAEEEAASLPDEGFVAFTANENCVLPDKSETASATSFPPSYESVTRGLSDRVNMRTSSSIQNEDEATSMELIAPGP

SEQ ID NO: 36 - protein, 1791 aa, Homo sapiens
  REGION 1..1791: note = misc_feature - Human Nav 1.9 (Figure 9)
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQAYELHQENKKPTSQRVQSVEIDMFSEDEPHLTIQDPRKKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 37 - protein, 36 aa, Homo sapiens
  REGION 1..36: note = misc_feature - Human Nax 1204-1239 (Figure 14B)
GGSNIFITVKQRKQYRRLKKLMYEDSQRPVPRPLNK

SEQ ID NO: 38 - protein, 36 aa, Homo sapiens
  REGION 1..36: note = misc_feature - Human Nav 1.1 1494-1529 (Figure 14B)
GGQDIFMTEEQKKYYNAMKKLGSKKPQKPIPRPGNK

SEQ ID NO: 39 - protein, 36 aa, Homo sapiens
  REGION 1..36: note = misc_feature - Human Nav 1.2 1484-1519 (Figure 14B)
GGQDIFMTEEQKKYYNAMKKLGSKKPQKPIPRPANK

SEQ ID NO: 40 - protein, 36 aa, Homo sapiens; identical to SEQ ID NO: 39
  REGION 1..36: note = misc_feature - Human Nav 1.3 1479-1514 (Figure 14B)

SEQ ID NO: 41 - protein, 36 aa, Homo sapiens
  REGION 1..36: note = misc_feature - Human Nav 1.4 1206-1241 (Figure 14B)
GGKDIFMTEEQKKYYNAMKKLGSKKPQKPIPRPQNK

SEQ ID NO: 42 - protein, 36 aa, Homo sapiens
  REGION 1..36: note = misc_feature - Human Nav 1.5 1481-1516 (Figure 14B)
GGQDIFMTEEQKKYYNAMKKLGSKKPQKPIPRPLNK

SEQ ID NO: 43 - protein, 36 aa, Homo sapiens; identical to SEQ ID NO: 42
  REGION 1..36: note = misc_feature - Human Nav 1.6 1475-1510 (Figure 14B)

SEQ ID NO: 44 - protein, 36 aa, Homo sapiens; identical to SEQ ID NO: 38
  REGION 1..36: note = misc_feature - Human Nav 1.7 1468-1503 (Figure 14B)

SEQ ID NO: 45 - protein, 36 aa, Homo sapiens; identical to SEQ ID NO: 42
  REGION 1..36: note = misc_feature - Human Nav 1.8 1429-1464 (Figure 14B)

SEQ ID NO: 46 - protein, 36 aa, Homo sapiens; identical to SEQ ID NO: 42
  REGION 1..36: note = misc_feature - Human Nav 1.9 1319-1354 (Figure 14B)

SEQ ID NO: 47 - protein, 31 aa, unidentified
  REGION 1..31: note = NaX_D1_S6 (Fig. 20)
GKVYMIFFVVVSFLFSFYMASLFLGILAMAY

SEQ ID NO: 48 - protein, 31 aa, unidentified
  REGION 1..31: note = NaV1.1_D1_S6 (Fig. 20)
GKTYMIFFVLVIFLGSFYLINLILAVVAMAY

SEQ ID NO: 49 - protein, 31 aa, unidentified; identical to SEQ ID NO: 48
  REGION 1..31: note = NaV1.2_D1_S6 (Fig. 20)

SEQ ID NO: 50 - protein, 31 aa, unidentified
  REGION 1..31: note = NaV1.3_D1_S6 (Fig. 20)
GKTYMIFFVLVIFLGSFYLVNLILAVVAMAY

SEQ ID NO: 51 - protein, 31 aa, unidentified
  REGION 1..31: note = NaV1.4_D1_S6 (Fig. 20)
GKTYMIFFVVIIFLGSFYLINLILAVVAMAY

SEQ ID NO: 52 - protein, 31 aa, unidentified
  REGION 1..31: note = NaV1.5_D1_S6 (Fig. 20)
GKIYMIFFMLVIFLGSFYLVNLILAVVAMAY

SEQ ID NO: 53 - protein, 31 aa, unidentified
  REGION 1..31: note = NaV1.6_D1_S6 (Fig. 20)
GKTYMIFFVLVIFVGSFYLVNLILAVVAMAY

SEQ ID NO: 54 - protein, 31 aa, unidentified
  REGION 1..31: note = NaV1.7_D1_S6 (Fig. 20)
GKTYMIFFVVVIFLGSFYLINLILAVVAMAY

SEQ ID NO: 55 - protein, 31 aa, unidentified
  REGION 1..31: note = NaV1.8_D1_S6 (Fig. 20)
GKIYMIFFVLVIFLGSFYLVNLILAVVTMAY

SEQ ID NO: 56 - protein, 31 aa, unidentified
  REGION 1..31: note = NaV1.9_D1_S6 (Fig. 20)
GLYSVFFFIVVIFLGSFYLINLTLAVVTMAY

SEQ ID NO: 57 - protein, 30 aa, unidentified
  REGION 1..30: note = NaX_D2_S6 (Fig. 20)
QSWCIPFYLMVILIGNLLVLYLFLALVSSF

SEQ ID NO: 58 - protein, 31 aa, unidentified
  REGION 1..31: note = NaV1.1_D2_S6 (Fig. 20)
QAMCLTVFMMVMVIGNLVVLNLFLALLLSSF

SEQ ID NO: 59 - protein, 31 aa, unidentified
  REGION 1..31: note = NaV1.2_D2_S6 (Fig. 20)
QTMCLTVFMMVMVIGNLVVLNLFLALLLSSF

SEQ ID NO: 60 - protein, 31 aa, unidentified
  REGION 1..31: note = NaV1.3_D2_S6 (Fig. 20)
QTMCLIVFMLVMVIGNLVVLNLFLALLLSSF

SEQ ID NO: 61 - protein, 31 aa, unidentified
  REGION 1..31: note = NaV1.4_D2_S6 (Fig. 20)
QAMCLTVFLMVMVIGNLVVLNLFLALLLSSF

SEQ ID NO: 62 - protein, 31 aa, unidentified
  REGION 1..31: note = NaV1.5_D2_S6 (Fig. 20)
QSLCLLVFLLVMVIGNLVVLNLFLALLLSSF

SEQ ID NO: 63 - protein, 31 aa, unidentified
  REGION 1..31: note = NaV1.6_D2_S6 (Fig. 20)
QAMCLIVFMMVMVIGNLVVLNLFLALLLSSF

SEQ ID NO: 64 - protein, 31 aa, unidentified
  REGION 1..31: note = NaV1.7_D2_S6 (Fig. 20)
QAMCLIVYMMVMVIGNLVVLNLFLALLLSSF

SEQ ID NO: 65 - protein, 31 aa, unidentified
  REGION 1..31: note = NaV1.8_D2_S6 (Fig. 20)
KSICLILFLTVMVLGNLVVLNLFIALLLNSF

SEQ ID NO: 66 - protein, 31 aa, unidentified
  REGION 1..31: note = NaV1.9_D2_S6 (Fig. 20)
SSLCVIVFILITVIGKLVVLNLFIALLLNSF

SEQ ID NO: 67 - protein, 31 aa, unidentified
  REGION 1..31: note = NaX_D3_S6 (Fig. 20)
NIYMYCYFINFIIFGVFLPLSMLITVIIDNF

SEQ ID NO: 68 - protein, 31 aa, unidentified
  REGION 1..31: note = NaV1.1_D3_S6 (Fig. 20)
SLYMYLYFVIFIIFGSFFTLNLFIGVIIDNF

SEQ ID NO: 69 - protein, 31 aa, unidentified
  REGION 1..31: note = NaV1.2_D3_S6 (Fig. 20)
NLYMYLYFVIFIIFGSFFTLNLFIGVIIDNF

SEQ ID NO: 70 - protein, 31 aa, unidentified; identical to SEQ ID NO: 69
  REGION 1..31: note = NaV1.3_D3_S6 (Fig. 20)

SEQ ID NO: 71 - protein, 31 aa, unidentified; identical to SEQ ID NO: 69
  REGION 1..31: note = NaV1.4_D3_S6 (Fig. 20)

SEQ ID NO: 72 - protein, 31 aa, unidentified
  REGION 1..31: note = NaV1.5_D3_S6 (Fig. 20)
NLYMYIYFVIFIIFGSFFTLNLFIGVIIDNF

SEQ ID NO: 73 - protein, 31 aa, unidentified
  REGION 1..31: note = NaV1.6_D3_S6 (Fig. 20)
NIYMYIYFVIFIIFGSFFTLNLFIGVIIDNF

SEQ ID NO: 74 - protein, 31 aa, unidentified
  REGION 1..31: note = NaV1.7_D3_S6 (Fig. 20)
SLYMYIYFVVFIIFGSFFTLNLFIGVIIDNF

SEQ ID NO: 75 - protein, 31 aa, unidentified
  REGION 1..31: note = NaV1.8_D3_S6 (Fig. 20)
NVYMYLYFVIFIIFGGFFTLNLFVGVIIDNF

SEQ ID NO: 76 - protein, 31 aa, unidentified
  REGION 1..31: note = NaV1.9_D3_S6 (Fig. 20)
NSLGYIYFVVFIIFGSFFTLNLFIGVIIDNF

SEQ ID NO: 77 - protein, 31 aa, unidentified
  REGION 1..31: note = NaX_D4_S6 (Fig. 20)
PSVGIFYFVSYILISWLIIVNMYIVVVMEFL

SEQ ID NO: 78 - protein, 31 aa, unidentified
  REGION 1..31: note = NaV1.1_D4_S6 (Fig. 20)
PSVGIFFFVSYIIISFLVVVNMYIAVILENF

SEQ ID NO: 79 - protein, 31 aa, unidentified; identical to SEQ ID NO: 78
  REGION 1..31: note = NaV1.2_D4_S6 (Fig. 20)

SEQ ID NO: 80 - protein, 31 aa, unidentified; identical to SEQ ID NO: 78
  REGION 1..31: note = NaV1.3_D4_S6 (Fig. 20)

SEQ ID NO: 81 - protein, 31 aa, unidentified
  REGION 1..31: note = NaV1.4_D4_S6 (Fig. 20)
PSIGICFFCSYIIISFLIVVNMYIAIILENF

SEQ ID NO: 82 - protein, 31 aa, unidentified
  REGION 1..31: note = NaV1.5_D4_S6 (Fig. 20)
PAVGILFFTTYIIISFLIVVNMYIAIILENF

SEQ ID NO: 83 - protein, 31 aa, unidentified
  REGION 1..31: note = NaV1.6_D4_S6 (Fig. 20)
PSVGIFFFVSYIIISFLIVVNMYIAIILENF

SEQ ID NO: 84 - protein, 31 aa, unidentified
  REGION 1..31: note = NaV1.7_D4_S6 (Fig. 20)
PSVGIFYFVSYIIISFLVVVNMYIAVILENF

SEQ ID NO: 85 - protein, 31 aa, unidentified
  REGION 1..31: note = NaV1.8_D4_S6 (Fig. 20)
PAVGIIFFTTYIIISFLIMVNMYIAVILENF

SEQ ID NO: 86 - protein, 31 aa, unidentified
  REGION 1..31: note = NaV1.9_D4_S6 (Fig. 20)
PGIATSYFVSYIIISFLIVVNMYIAVILENF

SEQ ID NO: 87 - protein, 16 aa, unidentified
  REGION 1..16: note = S4-gating charge
PTLQTARTLRILKIIP

SEQ ID NO: 88 - protein, 16 aa, unidentified
  REGION 1..16: note = S4-gating charge
ALLRLFRMLRIFKLGK

SEQ ID NO: 89 - protein, 17 aa, unidentified
  REGION 1..17: note = S4-gating charge
KPLISMKFLRPLRVLSQ

SEQ ID NO: 90 - protein, 22 aa, unidentified
  REGION 1..22: note = S4-gating charge
QLILLSRIIHMLRLGKGPKVFH